(19) United States
(12) Patent Application Publication
(10) Pub. No.: US 2011/0008832 A1
(43) Pub. Date: Jan. 13, 2011

(54) METHODS FOR STABLY RETAINING FOREIGN GENES IN CELLS

(76) Inventors: Yuji Hatada, Kanagawa (JP); Yukari Ohta, Kanagawa (JP); Yuko Hidaka, Kanagawa (JP); Nobuyuki Nakamura, Kanagawa (JP)

Correspondence Address: JACOBSON HOLMAN PLLC, 400 SEVENTH STREET N.W., SUITE 600, WASHINGTON, DC, 20004, US

(21) Appl. No.: 12/452,645

(22) PCT Filed: Jul. 9, 2008
(86) PCT No.: PCT/JP2008/062391

§ 371 Date: Jul. 26, 2010

(30) Foreign Application Priority Data
Jul. 13, 2007 (JP) 2007-183931

(51) Int. Cl. C12P 21/00; C12N 15/63; C12N 1/21; C12N 1/16; C12N 5/10

(52) U.S. Cl. 435/69.1; 435/320.1; 435/252.3; 435/254.2; 435/325; 435/419; 435/252.31; 435/471; 435/455; 435/468

(57) ABSTRACT

A method for preparing a protein or peptide encoded by a foreign gene by expressing a foreign gene, which comprises the steps of: preparing a recombinant vector comprising in an expressible state a gene encoding an aminoacyl-tRNA synthetase, in which a desired foreign gene has been inserted in an expressible state; preparing a mutant host cell or the like in which a chromosomal gene encoding an aminoacyl-tRNA synthetase has been knocked out; transforming the mutant host cell with the recombinant vector to obtain a transformant; and culturing the transformant to prepare the protein or peptide encoded by the foreign gene. It becomes possible to provide a novel means for permitting the retention of a recombinant DNA cloning vector without employing an antibiotic and without limiting the composition of the medium; and a method for preparing a protein or peptide encoded by a foreign gene by using the means.

### TECHNICAL FIELD

The present invention relates to methods for stably retaining foreign genes within cells. More specifically, the present invention relates to a recombinant vector comprising a gene encoding an aminoacyl-tRNA synthetase and a mutant host cell employed to express a foreign gene other than a gene encoding an aminoacyl-tRNA synthetase, that are employed in the above methods; and methods of preparing a protein or a peptide encoded by a foreign gene by expressing a foreign gene other than a gene encoding an aminoacyl-tRNA synthetase. As a cross-reference to a related patent application, the present application claims priority under Japanese Patent Application No. 2007-183931 filed on Jul. 13, 2007, the entire contents of which are hereby incorporated by reference.

### BACKGROUND ART

In attempts to produce target molecules with transformed cells obtained by the practical application of recombinant DNA techniques, there are problems in that the presence of an extrachromosomal gene in a transformed cells (that is, the targeted foreign gene contained therein) is generally highly unstable and the production of the targeted molecule is extremely difficult.

The cloning and expression vectors commonly employed in laboratories are normally multicopy recombinant vectors the stable transmission of which to subsequent generations is ensured by a large number of recombinant vectors for a single cell genome. However, the introduction of foreign genes into these recombinant vectors imparts various degrees of instability during the growth cycle of the cells. In industrial production processes, 1,000 L of culture product is required in some cases, necessitating 10¹⁶ or more cells following a succession of 50 generations or more. Accordingly, to achieve the reliable presence of the recombinant vector within the cell, and thus expression of the foreign gene, it is desirable to stabilize the recombinant vector in the cell through to the end of culturing in the fermentation vat.

There are several known methods of stabilizing a recombinant vector comprising a foreign gene in a cell.

One method consists of incorporating a gene imparting resistance to an antibiotic into a recombinant vector and adding a suitable antibiotic to the culture medium. Those cells having the recombinant vector comprising the gene for resistance to the antibiotic are selected, and those cells that do not have the recombinant vector are not selected, thereby eliminating them.

The stabilization of a recombinant vector by incorporating a gene imparting resistance to an antibiotic is regularly conducted in the laboratory. However, for the following reasons, it is undesirable in production on an industrial scale:
(i) the use of bacterial strains that are resistant to antibiotics is potentially dangerous to the environment;
(ii) the quantity of antibiotic required in the culture significantly increases production costs; and
(iii) the use of antibiotics is to be avoided in the production of substances intended for use by humans and in animals.

The method of supplementing a nutritional requirement mutation in a chromosome is also a known recombinant vector stabilization method (see Genetics. 1989 May; 122 (1): 19-27 (also referred to as Nonpatent Document 1 hereinafter) and Curr Genet. 1989 September; 16 (3): 159-63 (also referred to as Nonpatent Document 2 hereinafter); these descriptions are specifically incorporated herein by reference). In this method, the composition of the fermentation medium is strictly limited and fermentation must be conducted without adding a nutrient required by the host cell to the medium. Further, through syntrophism, it is possible for the cells to grow even once the recombinant vector has disappeared.

### DISCLOSURE OF INVENTION

### Problem to be Solved by the Invention

Thus, the two above-described selection methods depend on special processing of the medium. Such limitations tend to increase the cost of the fermentation process and restrict the freedom of choice in enhancing production efficiency.

Accordingly, there is a strong need for a selection method permitting the retention of the recombinant DNA cloning vector without using antibiotics and without restricting the composition of the medium.

Accordingly, the present invention has for its object to provide a new means of retaining a recombinant DNA cloning vector without the use of an antibiotic and without restrictions, and a method of preparing a protein or a peptide encoded by a foreign gene using this means.

### Means of Solving the Problem

Extensive research was conducted to solve the above problems. The present invention was devised on the basis of the discovery that a recombinant DNA cloning vector could be retained based on the complementarity between an extrachromosomal gene comprising an aminoacyl-tRNA synthetase gene and a chromosomal mutant host cell in which aminoacyl-tRNA synthetase activity had been knocked out, without using an antibiotic and without restricting the composition of the medium. The present invention is based on ensuring the survival of only those cells comprising an extrachromosomal gene containing an aminoacyl-tRNA synthetase gene.

The present invention is as follows:
(1) A recombinant vector, comprising a site permitting the insertion in an expressible state of a foreign gene other than a gene encoding an aminoacyl-tRNA synthetase and comprising a gene encoding aminoacyl-tRNA synthetase in an expressible state, for use in a mutant host in which a chromosomal gene encoding an aminoacyl-tRNA synthetase has been knocked out, or in a mutant host in which expression of a chromosomal gene encoding an aminoacyl-tRNA synthetase has been diminished to a degree to which the host cell cannot grow.
(2) The recombinant vector according to (1), wherein said aminoacyl-tRNA synthetase is tryptophanyl-tRNA synthetase, alanyl-tRNA synthetase, arginyl-tRNA synthetase, asparginyl-tRNA synthetase, aspartyl-tRNA synthetase, cysteinyl-tRNA synthetase, glutamine-tRNA synthetase, glutamate-tRNA synthetase, glycine-tRNA synthetase, histidyl-tRNA synthetase, isoleucyl-tRNA synthetase, leucyl-tRNA synthetase, lysine-tRNA synthetase, methionyl-tRNA synthetase, phenylalanine-tRNA synthetase, prolyl-tRNA synthetase, seryl-tRNA synthetase, threonyl-tRNA synthetase, tyrosyl-tRNA synthetase, or valyl-tRNA synthetase.
(3) The recombinant vector according to (1) or (2), wherein said recombinant vector is in the form of a plasmid, bacteriophage, or retrotransposon.
(4) A mutant host cell in which a chromosomal gene encoding an aminoacyl-tRNA synthetase has been knocked out, or a mutant host in which the expression of a chromosomal gene encoding an aminoacyl-tRNA synthetase has been diminished to a degree where the host cell cannot grow, that is used for transformation by a recombinant vector comprising a site permitting the insertion in an expressible state of a foreign gene other than a gene encoding an aminoacyl-tRNA synthetase and comprising in an expressible state a gene encoding an aminoacyl-tRNA synthetase.
(5) The mutant host cell according to (4), wherein said aminoacyl-tRNA synthetase is tryptophanyl-tRNA synthetase, alanyl-tRNA synthetase, arginyl-tRNA synthetase, asparginyl-tRNA synthetase, aspartyl-tRNA synthetase, cysteinyl-tRNA synthetase, glutamine-tRNA synthetase, glutamate-tRNA synthetase, glycine-tRNA synthetase, histidyl-tRNA synthetase, isoleucyl-tRNA synthetase, leucyl-tRNA synthetase, lysine-tRNA synthetase, methionyl-tRNA synthetase, phenylalanine-tRNA synthetase, prolyl-tRNA synthetase, seryl-tRNA synthetase, threonyl-tRNA synthetase, tyrosyl-tRNA synthetase, or valyl-tRNA synthetase.
(6) The mutant host cell according to (4) or (5), wherein the host cell into which a mutation is introduced is a bacterium, yeast, animal cell, or plant cell.
(7) The mutant host cell according to (4) or (5), wherein the host cell into which a mutation is introduced is a bacterium or yeast.
(8) The mutant host cell according to (6) or (7), wherein the bacterium is a bacterium of the genus Bacillus.
(9) A method of preparing a protein or peptide encoded by a foreign gene by expressing a foreign gene other than a gene encoding an aminoacyl-tRNA synthetase, comprising:

a step of preparing a recombinant vector, comprising a site permitting the insertion in an expressible state of a foreign gene and comprising in an expressible state a gene encoding an aminoacyl-tRNA synthetase, in which a desired foreign gene has been inserted at the site permitting the insertion in an expressible state of a foreign gene;

a step of preparing a mutant host cell in which a chromosomal gene encoding an aminoacyl-tRNA synthetase has been knocked out or a mutant host cell in which the expression of a chromosomal gene encoding an aminoacyl-tRNA synthetase has been diminished to a degree where the host cell cannot grow;

a step of transforming the mutant host cell with the recombinant vector to obtain a transformant; and

a step of culturing the transformant to prepare the protein or peptide encoded by the foreign gene.
(10) A method of causing a targeted recombinant vector to be stably retained within a host cell, comprising:

knocking out, or diminishing to a degree where the host cell cannot grow, a chromosomal gene encoding an aminoacyl-tRNA synthetase contained in the host cell, to convert the host cell to a mutant host cell;

incorporating in an expressible state a gene encoding an aminoacyl-tRNA synthetase into the targeted recombinant vector; and

transforming the mutant host cell with said targeted recombinant vector containing the gene encoding an aminoacyl-tRNA synthetase in an expressible state.
(11) The method according to (10), wherein the recombinant vector comprises a site permitting the insertion in an expressible state of a foreign gene other than a gene encoding an aminoacyl-tRNA synthetase.
(12) The method according to any one of (9) to (11), wherein said aminoacyl-tRNA synthetase is tryptophanyl-tRNA synthetase, alanyl-tRNA synthetase, arginyl-tRNA synthetase, asparginyl-tRNA synthetase, aspartyl-tRNA synthetase, cysteinyl-tRNA synthetase, glutamine-tRNA synthetase, glutamate-tRNA synthetase, glycine-tRNA synthetase, histidyl-tRNA synthetase, isoleucyl-tRNA synthetase, leucyl-tRNA synthetase, lysine-tRNA synthetase, methionyl-tRNA synthetase, phenylalanine-tRNA synthetase, prolyl-tRNA synthetase, seryl-tRNA synthetase, threonyl-tRNA synthetase, tyrosyl-tRNA synthetase, or valyl-tRNA synthetase.
(13) The method according to any one of (9) to (12), wherein said host cell into which a mutation is incorporated is a bacterium, yeast, animal cell, or plant cell.
(14) The method according to any one of (9) to (12), wherein said host cell into which a mutation is incorporated is a bacterium or yeast.
(15) The method according to (13) or (14), wherein said bacterium is a bacterium of the genus Bacillus.
(16) The method according to any one of (9) to (15), wherein the protein encoded by the foreign gene is an enzyme selected from the group consisting of oxidoreductases, transferases, hydrolases, phosphorylases, lyases, isomerases, ligases/synthetases, and modifying enzymes.
(17) Use of the recombinant vector according to any one of (1) to (3) to prepare a protein or peptide encoded by a foreign gene.
(18) Use of the mutant host cell according to any one of (4) to (8) to prepare a protein or peptide encoded by a foreign gene.

### Effect of the Invention

The present invention permits the retention of a recombinant DNA cloning vector without employing an antibiotic and without limiting the composition of the medium. As a result, it is possible to stabilize the recombinant vector in the cell through the end of cultivation in a fermentation vat, even in industrial production.

### BEST MODES OF CARRYING OUT THE INVENTION

### [The Recombinant Vector]

The present invention relates to a recombinant vector (1) comprising in an expressible state a gene encoding an aminoacyl-tRNA synthetase, (2) comprising a site permitting the insertion in an expressible state of a foreign gene other than a gene encoding an aminoacyl-tRNA synthetase, (3) that is used in a mutant host in which a chromosomal gene encoding an aminoacyl-tRNA synthetase has been knocked out or in a mutant host in which the expression of a chromosomal gene encoding an aminoacyl-tRNA synthetase has been diminished to a degree preventing growth of the host cell.
(1) The recombinant vector comprising in an expressible state a gene encoding an aminoacyl-tRNA synthetase.

The recombinant vector of the present invention comprises in an expressible state a gene encoding an aminoacyl-tRNA synthetase. The recombinant vector is used to modify a host cell by incorporating in an expressible state the gene encoding an aminoacyl-tRNA synthetase into a desired host cell. Further, the recombinant vector of the present invention need only have a form permitting the transformation of the host cell; examples are recombinant vectors in the form of plasmids, bacteriophages, and retrotransposons.

In a manner corresponding to the 20 amino acids, there exist 20 types of aminoacyl-tRNA synthetases (some of which come in multiple forms). For example, the aminoacyl-tRNA synthetase corresponding to alanine is called alanyl-tRNA (alanyl-tRNA synthetase or alanine-tRNA synthetase). Specific examples of aminoacyl-tRNA synthetases are tryptophanyl-tRNA synthetase, alanyl-tRNA synthetase, arginyl-tRNA synthetase, asparginyl-tRNA synthetase, aspartyl-tRNA synthetase, cysteinyl-tRNA synthetase, glutamine-tRNA synthetase, glutamate-tRNA synthetase, glycine-tRNA synthetase, histidyl-tRNA synthetase, isoleucyl-tRNA synthetase, leucyl-tRNA synthetase, lysine-tRNA synthetase, methionyl-tRNA synthetase, phenylalanine-tRNA synthetase, prolyl-tRNA synthetase, seryl-tRNA synthetase, threonyl-tRNA synthetase, tyrosyl-tRNA synthetase, and valyl-tRNA synthetase.

The aminoacyl-tRNA synthetase corresponding to tryptophan is called tryptophanyl-tRNA synthetase. Cell strains lacking tryptophanyl-tRNA synthetase cannot synthesize proteins containing tryptophan and thus cannot proliferate.

Aminoacyl-tRNA synthetase genes are contained in all living organisms, from prokaryotes to eukaryotes. For example, Escherichia coli contains: alanyl-tRNA synthetase (SEQ ID NO: 1), arginyl-tRNA synthetase (SEQ ID NO: 2), asparaginyl-tRNA synthetase (SEQ ID NO: 3), aspartyl-tRNA synthetase (SEQ ID NO: 4), cysteinyl-tRNA synthetase (SEQ ID NO: 5), glutamine-tRNA synthetase (SEQ ID NO: 6), glutamate-tRNA synthetase (SEQ ID NO: 7), glycine-tRNA synthetase, alpha subunit (SEQ ID NO: 8), glycine-tRNA synthetase, beta subunit (SEQ ID NO: 9), histidyl-tRNA synthetase (SEQ ID NO: 10), isoleucyl-tRNA synthetase (SEQ ID NO: 11), leucyl-tRNA synthetase (SEQ ID NO: 12), lysine-tRNA synthetase, constitutive (SEQ ID NO: 13), lysine-tRNA synthetase, inducible (SEQ ID NO: 14), methionyl-tRNA synthetase (SEQ ID NO: 15), phenylalanine-tRNA synthetase, alpha subunit (SEQ ID NO: 16), phenylalanine-tRNA synthetase, beta subunit (SEQ ID NO: 17), predicted lysyl-tRNA synthetase (SEQ ID NO: 18), prolyl-tRNA synthetase (SEQ ID NO: 19), seryl-tRNA synthetase, also charges selenocysteinyl-tRNA with serine (SEQ ID NO: 20), threonyl-tRNA synthetase (SEQ ID NO: 21), tryptophanyl-tRNA synthetase (SEQ ID NO: 22), tyrosyl-tRNA synthetase (SEQ ID NO: 23), and valyl-tRNA synthetase (SEQ ID NO: 24).

Bacillus subtilis is known to contain alanyl-tRNA synthetase (SEQ ID NO: 25), arginyl-tRNA synthetase (SEQ ID NO: 26, asparaginyl-tRNA synthetase (SEQ ID NO: 27), aspartyl-tRNA synthetase (SEQ ID NO: 28), cysteinyl-tRNA synthetase (SEQ ID NO: 29), glutamyl-tRNA synthetase (SEQ ID NO: 30), glutamyl-tRNA amidotransferase, subunit a (SEQ ID NO: 31), glycine-tRNA synthetase, alpha subunit (SEQ ID NO: 32), glycine-tRNA synthetase, beta subunit (SEQ ID NO: 33), histidyl-tRNA synthetase, hisS (SEQ ID NO: 34), isoleucyl-tRNA synthetase (SEQ ID NO: 35), leucyl-tRNA synthetase (SEQ ID NO: 36), lysine-tRNA synthetase, constitutive (SEQ ID NO: 37), histidyl-tRNA synthetase, hisZ (SEQ ID NO: 38), methionyl-tRNA synthetase (SEQ ID NO: 39), phenylalanine-tRNA synthetase, alpha subunit (SEQ ID NO: 40), phenylalanine-tRNA synthetase, beta subunit (SEQ ID NO: 41), similar to phenylalanine-tRNA synthetase, ytpR (SEQ ID NO: 42), prolyl-tRNA synthetase (SEQ ID NO: 43), seryl-tRNA synthetase, also charges selenocysteinyl-tRNA with serine (SEQ ID NO: 44), threonyl-tRNA synthetase, major (SEQ ID NO: 45), threonyl-tRNA synthetase, minor (SEQ ID NO: 46), tryptophanyl-tRNA synthetase (SEQ ID NO: 47), tyrosyl-tRNA synthetase, major (SEQ ID NO: 48), tyrosyl-tRNA synthetase, minor (SEQ ID NO: 49), valyl-tRNA synthetase (SEQ ID NO: 50), glutamyl-tRNA amidotransferase, subunit b (SEQ ID NO: 51), glutamyl-tRNA amidotransferase, subunit c (SEQ ID NO: 52), and the like.

The recombinant vector of the present invention comprises in an expressible state a gene encoding an aminoacyl-tRNA synthetase. Accordingly, when employing a bacterium such as Escherichia coli or Bacillus subtilis as a host to create an expressible state for a gene other than an aminoacyl-tRNA synthetase gene, the recombinant vector will generally comprise, for example, promoter and operator regions (including promoter, operator, and ribosome-binding regions (an SD region)), a start codon, DNA encoding the protein the production of which is targeted, a stop codon, a terminator region, and a plasmid-replicable unit. When employing an animal cell or a eukaryote such as yeast as the host cell, there will generally be a promoter, a start codon, DNA encoding a signal peptide, DNA encoding the protein the production of which is targeted, a stop codon, and the like. As needed, the recombinant vector of the present invention may also comprise cis elements such as enhancers, nontranslatable regions on the 5′ or 3′ end of the DNA encoding the protein the production of which is targeted, splicing joints, polyadenylation sites, replicable units, homologous regions, and selection markers. These elements are not specifically limited other than that they correspond to the host being used to express the gene encoding the protein the production of which is targeted, and may be selected based on common technical knowledge.

The selection marker is not specifically limited. When the host being used to express the gene is a bacterium, examples are genes imparting drug resistance (such as ampicillin-resistance genes, neomycin-resistance genes, cycloheximide-resistance genes, and tetramycin-resistance genes). When the host is not a bacterium, but is a yeast cell, for example, various known selection markers can be employed, including nutritional requirement genes (such as HIS4, URA3, LEU2, and ARG4).
(2) The recombinant vector of the present invention comprises a site permitting the insertion in an expressible state of a gene other than a gene encoding an aminoacyl-tRNA synthetase. This site, as set forth further below, permits the insertion in an expressible state of a foreign gene encoding the protein that is to be produced into the host cell of the present invention.

The phrase “permits the insertion of a gene in an expressible state” has the following meaning. Generally, the expression of a gene encoding a protein requires that information about the gene in the form of an RNA polymerase recognition site for transcription to mRNA and a ribosome-binding site for translating mRNA information into a peptide be present upstream from the start codon of the gene encoding the protein. In brief, the “presence of a gene in an expressible state” means that the gene, from start codon to end codon, must be positioned so that the sequence required for transcription to mRNA is present upstream from the start codon and the sequence required for translation of the mRNA information into a peptide is present upstream from the start codon. The “start codon” referred to here is not limited to the original start codon sequence of the gene on the chromosome, but may be any codon functioning as a start codon. The “ribosome binding-site” referred to here is not limited to a sequence having the consensus sequence consisting of 5′-aaaggagg-3′ of a prokaryote, for example, but can be any sequence that is recognized by a ribosome. It is not limited to the sequences of the original ribosome-binding sites of the gene on the chromosome; any sequence recognizable by a ribosome may be employed.

The recombinant vector of the present invention can be constructed according to the usual methods employed in DNA recombination, such as the method described in Molecular Cloning (1989) (Cold Spring Harbor Lab). The description given in this document is specifically incorporated herein by reference.

The following vectors may be employed. Examples of plasmid vectors are pRS413, pRS415, pRS416, YCp50, pAUR112, pAUR123, and other YCp-type Escherichia coli-yeast shuttle vectors; pRS403, pRS404, pRS405, pRS406, pAUR101, pAUR135, and other Ylp-type Escherichia coli-yeast shuttle vectors; plasmids derived from Escherichia coli (such as pBR322, pBR325, pUC18, pUC19, pUC119, pTV118N, pTV119N, pBluescript, pHSG298, pHSG396, pTrc99A, and other ColE plasmids; pACYC177, pACYC184, and other p1A plasmids; pMW118, pMW119, pMW218, pMW219, and other pSC101 plasmids); plasmids derived from Bacillus subtilis (such as pUB110 and pTP5); and pHY300PLK and other Escherichia coli-Bacillus subtilis shuttle vectors. Examples of phage vectors are lambda-phages (such as Charon 4A, Charon 21A, EMBL4, lambdagt100, gt11, zap); phiX174; M13mp18; and M13mp19. An example of a retrotransposon is Ty factor. Examples of expression vectors expressed as fused proteins that can be employed are the pGEX series (made by Pharmacia) and the pMAL series (made by Biolabs).
(3) The recombinant vector of the present invention is employed in a mutant host in which a chromosomal gene encoding an aminoacyl-tRNA synthetase has been knocked out, or in a mutant host in which the expression of a chromosomal gene encoding an aminoacyl-tRNA synthetase has been diminished to a degree where the host cell cannot grow. The mutant host will be described further below.

### [The Host Cell (Mutant Host)]

The present invention relates to (4) a mutant host cell in which a chromosomal gene encoding an aminoacyl-tRNA synthetase has been knocked out, or a mutant host in which expression of a chromosomal gene encoding an aminoacyl-tRNA synthetase has been diminished to a degree where the host cell cannot grow, that is employed for transformation by (5) a recombinant vector comprising a site permitting the insertion in an expressible state of a foreign gene other than a gene encoding an aminoacyl-tRNA synthetase and (6) comprising in an expressible state a gene encoding an aminoacyl-tRNA synthetase.
(4) The host cell prior to mutation of the mutant host cell in the present invention is, for example, a bacterium such as Escherichia coli or Bacillus subtilis; a yeast such as Saccharomyces cerevisiae, Saccharomyces pombe, or Pichia pastoris; an insect cell such as sf9 or sf21; a COS cell; a Chinese hamster ovarian cell (CHO cell), or some other animal cell; or a plant cell such as tobacco. Bacteria such as Escherichia coli and Bacillus subtilis, and yeast, are preferred.

Generally, a cell has chromosomal genes encoding aminoacyl-tRNA synthetases for its own proliferation. By contrast, in the present invention, the chromosomal gene encoding an aminoacyl-tRNA synthetase originally present in the cell is knocked out or the expression of a chromosomal gene encoding an aminoacyl-tRNA synthetase is diminished to a degree where growth of the host cell is prevented. A chromosomal gene encoding an aminoacyl-tRNA synthetase can be knocked out, or its expression diminished, by the following methods.

### [Gene Knockout (Deactivation)]

The term “deactivation” includes methods of impeding the functional expression of one or more chromosomal genes. Deactivation is conducted by knocking out, substituting (by mutation, for example), blocking, inserting into, and/or the like the nucleic acid gene sequence. A number of forms of implementation include the deactivation of one or more genes by causing deactivation in a mutant microorganism, desirably in a stable and irreversible fashion.

### (Deactivation by Insertion)

The term “insertion sequence” employed here refers to a DNA sequence that is introduced into the chromosome of a microorganism. In a number of forms of implementation, the insertion sequence is already present in the genome of the cell that is being transformed. A sequence that is not present (that is, a homologous or heterogenous sequence) can also be employed. In other forms of implementation, the insertion sequence may contain a selection marker. In still other forms of implementation, the insertion sequence may contain two homology boxes.

The term “homology box” employed here refers to a nucleic acid sequence that is homologous to the chromosomal sequence of a microorganism. More specifically, a homology box is an upstream or downstream region having about 80 to 100 percent sequence homology, about 90 to 100 percent sequence homology, or about 95 to 100 percent sequence homology with a coding region or gene adjacent to a gene being deactivated in accordance with the present invention. The purpose of such a sequence is to replace a portion of the chromosome of a microorganism. Although not a limitation of the present invention, a homology box contains about 1 base pair (bp) to 200 kilobases (kb). A homology box desirably contains about 1 bp to 10.0 kb, 1 bp to 5.0 kb, 1 bp to 2.5 kb, 1 bp to 1.0 kb, and 0.25 kb to 2.5 kb. Further, a homology box contains about 10.0 kb, 5.0 kb, 2.5 kb, 2.0 kb, 1.5 kb, 1.0 kb, 0.5 kb, 0.25 kb, and 0.1 kb. In a number of forms of implementation, the 5′ and 3′ ends of a selection marker are adjacent to homology boxes; here, the homology boxes include the nucleic acid sequences that are immediately adjacent to the regions encoding the particular gene.

The term “selection marker” employed here refers to a nucleotide sequence capable of being expressed in the host cell. The expression of a selection marker in the presence of a corresponding selection factor, or in the absence of a required nutrient, allows a cell containing the gene being expressed to grow. The terms “selectable marker” and “selection marker” used here refer to nucleic acid (such as a gene) that can be expressed in a host cell to facilitate the selection of hosts containing a vector. Although not a limitation, examples of such selection markers are antibiotics. Accordingly, a “selection marker” refers to a gene imparting a sign that the targeted insertion DNA has been incorporated into the host cell, or that some other reaction has occurred. Normally, a selection marker refers to a gene imparting bacterial resistance or a metabolic advantage to a host cell that makes it possible to distinguish cells containing foreign DNA from cells that have not picked up the foreign sequence during transformation. Examples are selection markers that impart resistance to antibiotics. (Examples are ampR, phleoR, specR, kanR, eryR, tetR, cmpR, and neoR. For example, see Guerot-Fleury, Gene, 167: 335-337 [1995]; Palmeros et al., Gene, 247: 255-265 [2000]; and Trieu-Cuot et al., Gene, 23: 331-341 [1983]. These descriptions are specifically incorporated herein by reference.) In a number of preferred forms of implementation, the present invention provides a gene imparting resistance to chloramphenicol (such as the gene present on pC194 and the resistance gene present in the genome of Bacillus lichenformis). Such a resistance gene is particularly useful in forms of implementation relating to chromosomal amplification of integrated plasmids, cassettes integrated onto chromosomes, and the present invention.

For example, when the gene being deactivated is the tryptophenyl-tRNA synthetase gene (trpS), the DNA structure contains a selection marker as an insertion sequence and comprises the trpS gene, which blocks its activity. The selection marker is inserted within the trpS code sequence portion. The DNA structure has essentially the same sequence as the trpS gene in the host chromosome. In double crossing over, the trpS gene is deactivated by insertion of the selection marker.

### (Deactivation by Knockout)

In a number of desirable forms of implementation, deactivation is achieved by knockout. In a number of desirable forms of implementation, the gene is knocked out by homologous recombination. For example, in a number of forms of implementation, when the gene being knocked out is trpS, a DNA structure containing insertion sequences having selection markers adjacent to the two sides of a homology box is employed. The homology box contains nucleotide sequences that are homologous to the nucleic acid flanking regions of the chromosomal trpS gene. The DNA structure matches the homologous sequence of the chromosome of the microorganism host. In double crossing over, the trpS gene is cut out of the host chromosome.

The term “knocking out” a gene that is employed here refers to knocking out an entire code sequence, knocking out a portion of a code sequence, or knocking out a code sequence containing a flanking region. Knocking out may be partial so long as the sequence remaining in the chromosome does not exhibit the bioactivity it had prior to being knocked out. The flanking regions of the code sequence can contain about 1 bp to about 500 bp on the 5′ and 3′ ends. The flanking regions can be larger than 500 bp, but in accordance with the present invention, desirably do not contain other genes that can be deactivated or knocked out. Finally, the gene that has been knocked out is essentially nonfunctional. Simply stated, the term “knocked out” is defined as a change in a nucleotide or amino acid sequence whereby one or more nucleotide or amino acid residue is eliminated (that is, absent).

The term “flanking sequence” employed here refers to a sequence that is upstream or downstream from a target sequence (for example, in genes A-B-C, gene B is flanked (positioned beside) gene sequences A and C). In a desirable form of implementation, the insertion sequence is positioned on both sides of a homology box. In a number of forms of implementation, the flanking sequence is present on only one side (3′ or 5′), but in a desirable form of implementation, it is positioned on both sides of the sequence. The sequences of each homology box are homologous to sequences in the chromosome of the microorganism. The purpose of these sequences is to integrate a new structure into the chromosome of the microorganism so that a portion of the chromosome of the microorganism is replaced by the insertion sequence. In a desirable form of implementation, the 5′ and 3′ ends of the selection marker are positioned beside the polynucleotide sequence containing a portion of the deactivated chromosomal fragment.

### (Deactivation by Mutation)

In another form of implementation, deactivation is produced by mutation of the gene. Various methods of gene mutation can be employed; the method is not limited. Examples are site-specific mutation, random mutation generation, and the gapped-duplex method. (For example, see Moring et al., Biotech. 2: 646 [1984], and Kramer et al., Nucleic Acids Res., 12: 9441 [1984]. These descriptions are specifically incorporated herein by reference).

In a desirable form of implementation, a mutation is produced by inducing mutation in a codon on the chromosome. In another form of implementation, the mutant DNA sequence has 40 percent or greater, 45 percent or greater, 50 percent or greater, 55 percent or greater, 60 percent or greater, 65 percent or greater, 70 percent or greater, 75 percent or greater, 80 percent or greater, 85 percent or greater, 90 percent or greater, 95 percent of greater, or 98 percent or greater, homology with the sequence of the wild form. In another form of implementation, the mutant DNA is produced in vivo by a known mutation-inducing procedure such as UV irradiation or a chemical mutagenic agent such as nitrosoguanidine.

In terms of chromosomal mutations that knock out aminoacyl-tRNA synthetase activity, the knocking out of an aminoacyl-tRNA synthetase gene itself is desirable. However, other genes that regulate the synthesis of aminoacyl-tRNA synthetases can be employed so long as an extrachromosomal gene retaining the corresponding gene is employed. In the case of a strain in which an aminoacyl-tRNA synthetase gene has been knocked out, the gene that must be inserted into the extrachromosomal gene corresponds to the particular aminoacyl-tRNA synthetase gene.

To avoid the possibility of the homologous recombination of an extrachromosomal gene having an aminoacyl-tRNA synthetase with a mutated aminoacyl-tRNA synthetase gene of the chromosome, the targeted aminoacyl-tRNA synthetase gene is desirably essentially deleted from the chromosome.

The recombinant vector of (5) and (6) is identical to the recombinant vector of (1) and (2) above.

### [Functions]

Since the mutant host cell in which a chromosomal gene encoding an aminoacyl-tRNA synthetase has been knocked out, or the mutant host in which the expression of a chromosomal gene encoding aminoacyl-tRNA synthetase has been diminished to a degree where the host cell cannot grow, cannot express the chromosomal gene encoding the aminoacyl-tRNA synthetase, it cannot proliferate as is.

By contrast, by incorporating a recombinant vector having a gene encoding the aminoacyl-tRNA synthetase in a state permitting expression, proliferation can be maintained. That is, knocking out the aminoacyl-tRNA synthetase can be complemented by incorporating the particular aminoacyl-tRNA synthetase onto the chromosomal gene. However, if the cell loses the particular extrachromosomal gene, the cell also becomes unable to proliferate.

Accordingly, in the mutant host cell of the present invention, given the above relation, a recombinant vector into which has been inserted a foreign gene can be stably retained in the host cell without incorporating an antibiotic into the medium and without eliminating or limiting nutrients satisfying specific nutritional requirements. Thus, the mutant host cell of the present invention affords the advantage of permitting application to any medium employed in production on an industrial scale.

Further, the mutant host cell of the present invention can be employed to express a foreign gene other than a gene encoding an aminoacyl-tRNA synthetase. The host cell of the present invention contains a recombinant vector comprising in an expressible state a gene encoding the above-described aminoacyl-tRNA synthetase. Further, this recombinant vector comprises a site permitting the insertion in an expressible state of a foreign gene. By inserting a desired foreign gene at this site, the recombinant vector into which the desired foreign gene has been inserted can be stably maintained in the host cell. Expression of the foreign gene can be induced to produce a desired protein encoded by the foreign gene.

### [The Method for Preparing a Protein or Peptide]

The present invention relates to a method for preparing a protein or peptide encoded by a foreign gene by expressing a foreign gene other than a gene encoding an aminoacyl-tRNA synthetase. This method comprises the following steps:
(10) preparing a recombinant vector, comprising a site permitting the insertion in an expressible state of a foreign gene and comprising in an expressible state a gene encoding an aminoacyl-tRNA synthetase, in which a desired foreign gene has been inserted at the site permitting the insertion in an expressible state of a foreign gene;
(11) preparing a mutant host cell in which a chromosomal gene encoding an aminoacyl-tRNA synthetase has been knocked out or a mutant host cell in which the expression of a chromosomal gene encoding an aminoacyl-tRNA synthetase has been diminished to a degree where the host cell cannot grow;
(12) transforming the mutant host cell with the recombinant vector to obtain a transformant; and
(13) culturing the transformant to prepare the protein or peptide encoded by the foreign gene.

### (10) The Step of Preparing a Recombinant Vector

In this step, a recombinant vector is prepared that comprises in an expressible state a gene encoding an aminoacyl-tRNA synthetase and that comprises a site permitting the insertion in an expressible state of a foreign gene, in which site a desired foreign gene has been inserted. The recombinant vector is the recombinant vector of the invention of the present application set forth above. In the preparing method of the present invention, a recombinant vector is prepared with a desired foreign gene inserted at the site of the recombinant vector permitting the insertion in an expressible state of a foreign gene. Specifically, a gene encoding an aminoacyl-tRNA synthetase and a desired foreign gene are inserted into a suitable vector. The genes can be inserted by the usual methods. Both genes are inserted in expressible states.

### [A General Description of the Insertion of the Two Genes]

The aminoacyl-tRNA synthetase gene and the foreign gene can be inserted into the recombinant vector by common methods of DNA recombination. For example, they can be inserted by the method described in Molecular Cloning (1989) (Cold Spring Harbor Lab.). The entire description of this document is specifically incorporated herein by reference. The individual genes can be inserted into the recombinant vector at essentially any position so long as they do not contribute to replication of the recombinant vector; the insertion positions will vary with the vector employed. Examples of recombinant vectors that can be employed are plasmids derived from Escherichia coli (such as pBR322, pBR325, pUC18, pUC19, pUC119, pTV118N, pTV119N, pBluescript, pHSG298, pHSG396, pTrc99A, and other ColE plasmids; pACYC177, pACYC184, and other p1A plasmids; and pMW118, pMW119, pMW218, pMW219, and other pSC101 plasmids); plasmids derived from Bacillus subtilis (such as pUB110 and pTP5); and pHY300PLK and other Escherichia coli-Bacillus subtilis shuttle vectors.

In the example of the pHY300PLK plasmid, which is an Escherichia coli-Bacillus subtilis shuttle vector, an aminoacyl-tRNA synthetase gene can be inserted at the EcoRI site, which is a multicloning site, and a foreign gene can be inserted at the BamHI site, which is also a multicloning site. The present example will be described in some detail below.

Using PCR amplification with the chromosomal DNA of an organism having a foreign gene as template, the foreign gene is amplified from its promoter site to its stop codon. A ligation reaction is then conducted with this gene and a DNA fragment obtained by digesting pHY300PLK with BamHI restriction enzyme. The ligation reaction mixture obtained is then used to transform Escherichia coli, transformants containing the plasmid in which a foreign gene has been inserted as targeted from the promoter site to the stop codon at the BamHI site of pHY300PLK are selected, and a plasmid is prepared from a transformant. Next, the plasmid thus prepared is digested with EcoRI restriction enzyme and cleaved. PCR amplification with chromosomal DNA of Bacillus subtilis strain 168 as template is used to amplify an aminoacyl-tRNA synthetase gene from its promoter site to its stop codon, which is subjected to a ligation reaction with the plasmid-derived DNA fragment with the structure that has been cleaved with EcoRI. The ligation reaction mixture obtained is used to transform Escherichia coli, transformants containing the plasmid in which an aminoacyl-tRNA synthetase gene has been inserted as targeted at the EcoRI site of the constructed plasmid are selected, and the targeted plasmid can be prepared from the transformants.

### (11) The Step of Preparing a Mutant Host Cell

In this step, a mutant host cell in which the chromosomal gene encoding an aminoacyl-tRNA synthetase has been knocked out, or a mutant host cell in which the expression of a chromosomal gene encoding an aminoacyl-tRNA synthetase has been diminished to a degree where the host cell cannot develop, is prepared. The mutant host cell is the mutant host cell of the present invention as set forth above. The method of preparation is as set forth below.

Examples of the procedure for preparing the mutant host cell include methods of eliminating or deactivating in a planned manner the target gene present on the host chromosome and imparting random gene elimination or deactivation mutations and then using suitable methods to analyze the genes or evaluate protein productivity.

It suffices to employ homologous recombination, for example, to eliminate or deactivate a target gene. That is, the host cell is caused to incorporate a circular recombinant plasmid or linear DNA fragment obtained by cloning a DNA fragment containing part of a targeted gene into a suitable plasmid vector. Homologous recombination in a partial region of the target gene cleaves the target gene on the parent microorganism genome, thereby deactivating it. Alternatively, a target gene that has been imparted with a deactivating mutation by base substitution, base insertion, or the like, or a linear DNA fragment or the like that contains the region outside a target gene but not the target region, is construction by a method such as PCR, incorporated into a parent microorganism cell, and subjected to double crossing over homologous recombination at two spots outside the location of the mutation in the target gene in the parent microorganism genome, or in two spots outside the target gene, to substitute the eliminated or deactivated gene fragment for the target gene on the genome.

### (12) The Step of Obtaining a Transformant

The mutant host cells prepared in step (11) are transformed with the recombinant vector prepared in step (10). The transformation of the host cells with the recombinant vector can also be implemented by the usual methods.

The method of incorporating (transforming) the recombinant vector into the host cells is not specifically limited. The transformation method, transfection method, competent cell method, electroporation, or the like can be suitably selected based on the type of host cell into which the recombinant vector is being introduced and on the form of the recombinant vector.

The term “host cell” employed here refers to a cell having the ability to function as a host or an expression medium for a newly introduced DNA sequence.

The form in which the recombinant vector is present within the host cell is not specifically limited. It can be inserted into the chromosome, incorporated by substitution, or present in the form of a plasmid.

The chromosomal gene encoding the aminoacyl-tRNA synthetase contained in the recombinant vector is the same gene as the chromosomal gene in the mutant host cell encoding the aminoacyl-tRNA synthetase that has been knocked out or the chromosomal gene in the mutant host cell encoding the aminoacyl-tRNA synthetase the expression of which has been diminished to a degree where the host cell is incapable of developing. For example, when the chromosomal gene encoding the aminoacyl-tRNA synthetase that has been knocked out or the chromosomal gene encoding the aminoacyl-tRNA synthetase the expression of which has been diminished to a degree where the cost cell is incapable of developing is the alanyl-tRNA synthetase gene in the mutant host cell, the gene contained in the recombinant vector is also the alanyl-tRNA synthetase gene.

### (13) The Step of Preparing a Protein or Peptide

The transformant obtained in step (12) is cultured to prepare a protein or peptide encoding the foreign gene. The transformant is cultured using a suitable medium and under suitable conditions based on the type of host cell. The host cell need only be capable of producing the protein or peptide encoded by the foreign gene, and may be of the wild type or a mutated form. Specific examples are bacteria of the genus Bacillus, such as Bacillus subtilis; bacteria of the genus Escherichia; bacteria of the genus Clostridium; and yeast. Of these, bacteria of the genus Bacillus are desirable. Bacillus subtilis is preferred because its entire genome is known, genetic engineering and genomal engineering techniques have been established, and it can be made to produce protein and secrete it externally.

In the present specification, the term “genus Bacillus” covers all the generally known strains contained in the genus Bacillus without limitation. For example, it means: Bacillus subtilis, Bacillus licheniformis, Bacillus lentus, Bacillus stearothermophilus, Bacillus alkalophilus, Bacillus amyloliquefaciens, Bacillus cereus, Bacillus pumilus, Bacillus clausii, Bacillus halodurans, Bacillus megaterium, Bacillus coagulans, Bacillus circulans, and Bacillus thuringiensis. Classification of the genus Bacillus is continuing, and strains that are classified in this genus are also included. For example, the genera Geobacillus, Alkalibacillus, Amphibacillus, Amylobacillus, Anoxybacillus, Goribacillus, Cerasibacillus, Gracilibacillus, Halolactibacillus, Halalkalibacillus, Filobacillus, Jeotgalibacillus, Salibacillus, Oceanobacillus, Marinibacillus, Lysinibacillus, Lentibacillus, Ureibacillus, Salinibacillus, Pontibacillus, Piscibacillus, Paraliobacillus, Virgibacillus, Salsuginibacillus, Tenuibacillus, Thalassobacillus, Thermalkalibacillus, and Tumebacillus exist. These are also included in the genus Bacillus in the present specification.

For example, when the host cell is Bacillus subtilis, it suffices for the culture medium employed to contain a suitable nitrogen source, carbon source, and minerals for growth of the Bacillus subtilis host of the present invention and production of the targeted protein. For example, when recombinant Bacillus subtilis strain 168 is used to produce a protein or peptide encoded by a foreign gene, a medium compounded with a carbon source in the form of a monosaccharide such as glucose or fructose, a disaccharide such as sucrose or maltose, or a polysaccharide such as soluble starch, and a medium compounded with a nitrogen source in the form of peptones, soybean extract, yeast extract, fish extract, corn steep liquor (CSL), or metal salts can be employed.

The pH of the medium need only be a pH falling within a range permitting growth of the recombinant organism employed. For example, in the case of Bacillus subtilis, the pH is suitably adjusted to 6.0 to 8.0, and the growth conditions are stirred culturing with ventilation or shaking for 2 to 7 days at 15 to 42° C., desirably 28 to 37° C.

The targeted protein or peptide is recovered after the above culturing. The targeted protein or polypeptide can be recovered by the usual methods. The targeted protein or polypeptide that is recovered can be purified by the usual methods as needed.

Examples of the targeted protein or peptide produced by the manufacturing method of the present invention are enzymes that are useful in foods, pharmaceuticals, cosmetics, detergents, textile processing, medical test reagents, and the like, as well as polypeptides and proteins such as physiologically active factors.

The targeted protein or peptide gene is not specifically limited, and includes enzymes employed in various industries, such as detergents, foods, textiles, feeds, chemicals, medical treatment, and diagnosis; as well as physiologically active peptides. Based on function, industrial enzymes include oxidoreductases, transferases, hydrolases, phosphorylases, lyases, isomerases, ligases/synthetases, and modifying enzymes. More specific examples are the genes of sugar-degrading enzymes such as cellulase, agarase, and gamma-cyclodextrin synthetase.

[Method of Causing a Recombinant Vector to be Stably Retained within a Host Cell]

The present invention relates to a method of causing a targeted recombinant vector to be stably retained in a host cell. This method comprises (20) to (22) below:
(20) knocking out, or diminishing to a degree where the host cell cannot grow, a chromosomal gene encoding an aminoacyl-tRNA synthetase contained in the host cell, to convert the host cell to a mutant host cell;
(21) incorporating in an expressible state a gene encoding an aminoacyl-tRNA synthetase into the targeted recombinant vector; and
(22) transforming the mutant host cell with the targeted recombinant vector containing the gene encoding an aminoacyl-tRNA synthetase in an expressible state.

(20) The method of converting a host cell to a mutant host cell is as stated in the description of mutant host cells above.

(21) The incorporation in an expressible state of a gene encoding an aminoacyl-tRNA synthetase into the targeted recombinant vector is also as stated in the description of the recombinant vector of the present invention. The fact that the recombinant vector has a site permitting the insertion in an expressible state of a foreign gene other than a gene encoding an aminoacyl-tRNA synthetase is also as set forth above.

(22) The transformation of the mutant host cell is also as stated in the description of the above-described method for preparing a protein or peptide.

The present invention includes a method of causing a targeted recombinant vector to be stably retained in a host cell. In this method, the mutant host cell of the present invention is employed as the host cell. The method of the present invention is one of stabilizing a extrachromosomal gene contained in a cell such as that set forth above. For example, the chromosomal mutation rendering an aminoacyl-tRNA synthetase deficient is an at least partial knockout of a particular aminoacyl-tRNA synthetase gene and the extrachromosomal gene is the particular aminoacyl-tRNA synthetase gene.

Based on the present invention, for example, a transformed cell strain in which an extrachromosomal gene is stable in non-limited medium can be obtained without requiring selection by an antibiotic or a medium with a limited content of a specific amino acid. Further, based on the method described in the present invention, cells that have lost the extrachromosomal gene cannot proliferate. Accordingly, the present invention relates to cells that have been modified by an aminoacyl-tRNA synthetase gene and an extrachromosomal gene that contains a factor that renders reliable the expression of an industrially useful protein. Further, the present invention relates to a method of manufacturing an industrially useful protein employing a transformed cell, characterized in that the cell that has been transformed by an extrachromosomal gene of the present invention is cultured in non-limited medium. The extrachromosomal gene retains the gene for the particular protein.

The term “protein gene” employed here means a chromosomal fragment of DNA relating to the production of a peptide chain that may or may not comprise a region preceding or succeeding the region encoding the protein.

The embodiments set forth below compare the stability of a plasmid comprising the tryptophanyl-tRNA synthetase gene in Bacillus subtilis having a deficiency in tryptophanyl-tRNA synthetase, which is one of aminoacyl-tRNA synthetases, to the stability of the same plasmid in Bacillus subtilis that does not have a deficiency in tryptophanyl-tRNA synthetase. They also compare the stability of a plasmid comprising the tryptophanyl-tRNA synthetase gene in Bacillus subtilis having a deficiency in tryptophanyl-tRNA synthetase, which is one of aminoacyl-tRNA synthetases, to the stability of the same plasmid not comprising the tryptophanyl-tRNA synthetase gene in Bacillus subtilis not having a deficiency in tryptophanyl-tRNA synthetase.

Examples of the use of recombinant vectors comprising the tryptophanyl-tRNA synthetase gene to express genes encoding cellulase and agarase transported in the same vector are also given.

### Embodiments

The present invention is described in greater detail below through embodiments.

### [Embodiment 1] The Introduction of an Extrachromosomal Gene Including the Tryptophanyl-tRNA Synthetase Gene into Bacillus Subtilis

The plasmid pDATS14, which is an extrachromosomal gene containing the tryptophanyl-tRNA synthetase gene, was constructed by the following method. First, primers A and B were used with a commonly employed Bacillus subtilis-Escherichia coli shuttle plasmid in the form of pHY300PLK (made by Yakult) as template to conduct PCR amplification. The amplified fragment obtained was digested with XhoI restriction enzyme and the two ends were joined to form a circular plasmid. Escherichia coli strain HB101 was transformed with this plasmid. Plasmid was prepared from the transformant and named pDA2. Additionally, primers C and D were used with the chromosome of Bacillus subtilis strain ISW1214 as template to obtain a DNA fragment comprising the tryptophanyl-tRNA synthetase gene and nearby regions. This DNA fragment was digested with XhoI restriction enzyme and spliced to the pDA2 that had been digested with XhoI restriction enzyme. This was then used to transform Escherichia coli strain HB101. Plasmid was prepared from the transformant obtained and named pDATS14. Using pDATS14, a mutant strain derived from Bacillus subtilis strain ISW1214 having deficiencies in two proteases (alkali protease E and neutral protease E) (see Reference Appl. Microbiol. Biotechnol 65: 583-592 (2004) Hatada, Y. et al., this description being incorporated herein in its entirety by reference) was transformed by the protoplast transformation method (see Reference Mol. Gen. Genet. 168: 111-115 (1979) Chang, S. and Cohen, S. N., this description being incorporated herein in its entirety by reference) to obtain a Bacillus subtilis transformant having an extrachromosomal tryptophanyl-tRNA synthetase gene.

### [Embodiment 2] The Construction of a DNA Fragment to Impair the Tryptophanyl-tRNA Synthetase Gene on the Chromosome of Bacillus Subtilis

An about 2.5 kb DNA fragment containing the tryptophanyl-tRNA synthetase gene and nearby regions was obtained using primers E and F with the chromosome of Bacillus subtilis strain ISW1214 as template. This was digested with the restriction enzymes EcoRI and SalI. It was then spliced with general purpose plasmid pUC18 that had been predigested with EcoRI and SalI to obtain the plasmid pTSAF. Using primers G and H with a general-purpose plasmid in the form of pC194 as template, an about 1.6 kb DNA fragment containing a Staphylococcus-derived chloramphenicol resistance gene and nearby areas was obtained. Using primers I and J with pTSAF as template, a PCR reaction was conducted to obtain a DNA fragment. This DNA fragment and the 1.6 kb DNA fragment containing the chloramphenicol resistance gene and nearby regions were digested with restriction enzymes BamHI and XbaI, spliced, and used to transform Escherichia coli strain HB101. Plasmid was prepared from the transformant and named tryptophanyl-tRNA synthetase gene-impairing plasmid pINTTS. Using primers K and L with pINTTS as template, a PCR reaction was conducted to obtain a linear tryptophanyl-tRNA synthetase gene-impairing DNA fragment.

### [Embodiment 3] Impairing the Tryptophanyl-tRNA Synthetase Gene on the Chromosome of Bacillus Subtilis

Using the above-described tryptophanyl-tRNA synthetase gene-impairing DNA fragment, the competent cell method was employed to further transform the above-described transformant containing the extrachromosomal tryptophanyl-tRNA synthetase gene, yielding a transformant with chloramphenicol resistance. Chromosomal DNA was prepared from the transformant. The chromosomal DNA was subjected to a PCR reaction using primers M and N or O and P. The fact that the region encoding the tryptophanyl-tRNA synthetase gene on the chromosome had been replaced with the chloramphenicol resistance gene derived from the DNA fragment for impairing the tryptophanyl-tRNA synthetase gene was confirmed. This bacterial strain, a tryptophanyl-tRNA synthetase gene knockout strain, was named strain DTS1451(pDATS14).

### [Embodiment 4] Introducing Plasmid Having a Kanamycin Resistance Gene into Strain DTS1451

Using primers C and D with the chromosome of Bacillus subtilis strain ISW1214 as template, a DNA fragment containing the tryptophanyl-tRNA synthetase gene and nearby regions was obtained. This DNA fragment was digested with XhoI restriction enzyme and spliced with pDA2 that had been predigested with the restriction enzyme XhoI. This was then used to transform Escherichia coli strain HB101. Plasmid was prepared from the transformant obtained and named pDATS13. Using primers Q and R with general purpose plasmid pUB110 as template, a DNA fragment containing the kanamycin resistance gene and nearby regions was obtained. This DNA fragment was digested with EcoRI, amplified by PCR using primers S and T with pDATS14 as template, and then spliced with the DNA fragment obtained by digestion with EcoRI. It was then used to transform Escherichia coli strain HB101. Plasmid was prepared from the transformant having kanamycin resistance obtained. The plasmid was named pDATSK. Using pDATSK, strain DTS1451 containing pDATS14 was transformed by the protoplast transformation method and selection was conducted in regenerated medium containing 30 microgram/mL of kanamycin (composition: 8 percent sodium succinate, 1 percent agar, 0.5 percent casamino acids, 0.5 percent yeast extract, 0.15 percent potassium dihydrogenphosphate, 0.35 percent dipotassium hydrogenphosphate, 0.5 percent glucose, 0.4 percent magnesium sulfate, 0.01 percent bovine serum albumin, 0.001 percent methionine, and 0.001 percent leucine). The transformants obtained were inoculated into kanamycin-containing LB agar medium and regenerated medium containing 7.5 micrograms/mL of tetracycline. A strain exhibiting resistance to kanamycin and sensitivity to tetracycline was named DTS1451(pDATSK).

### [Embodiment 5] Producing Cellulase in DTS1451

A PCR reaction was conducted using primers U and V with the chromosome of Bacillus akibai strain 1139 (JCM 9157T), a cellulase-producing bacterium (see J. Gen. Microbiol. 1986, 132, 2329-2335, Fukumori et al.; Int J Syst Evol Microbiol. (2005) 55: 2309-15. Nogi, Y. et al.; these descriptions are hereby incorporated in their entirety by reference) as template to obtain a DNA fragment containing cellulase and nearby regions. This DNA fragment and pDATS13 were digested with the restriction enzyme BamHI and then spliced to obtain plasmid pDATSC1. This plasmid was employed to transform DTS1451(pDATSK) and selection was conducted with a regenerated medium containing tetracycline. The transformants obtained were inoculated onto kanamycin-containing LB agar medium and regenerated medium containing 7.5 microgram/mL of tetracycline. A strain that exhibited cellulase activity, was sensitive to kanamycin, and was resistant to tetracycline was named DTS1451(pDATSC1). DTS1451(pDATSC1) was cultured with stirring at 130 rpm for 72 hours at 30° C. in PPS medium (3 percent polypeptone S, 0.5 percent fish extract, 0.05 percent yeast extract, 0.1 percent potassium dihydrogenphosphate, 4 percent maltose, 0.02 percent magnesium sulfate, and 0.05 percent calcium chloride) (to which no antibiotic was added). The cellulase activity of the supernatant of the culture obtained was measured. As a result, substantial production of cellulase of about one gram per liter of culture solution was confirmed.

### [Embodiment 6] The Retention of Plasmid pDATSC1 in Strain DTS1451

Strain DTS1451 containing pDATSC1 was inoculated into LB media to which 7.5 micrograms/mL of tetracycline had been added or into which no tetracycline had been added (1 percent polypeptone, 0.5 percent yeast extract, and 1 percent sodium chloride) and cultured with stirring at 130 rpm for 24 hours at 30° C. A 10 microliter quantity of the culture solution was collected and transplanted to another 100 mL of LB medium. Culturing was conducted for another 24 hours. A 10 microliter quantity of the culture solution was collected, transplanted to another 100 mL of LB medium, and similarly cultured for 24 hours. Subsequently, operations were conducted to recover the plasmid from the cultured cells. As a result, in the DTS1451 strain containing pDATSC1, regardless of whether tetracycline was added or not, no change was observed in the content of plasmid per the culture solution. In the mutant strain of Bacillus subtilis strain ISW1214 having pDATSC1 and not deficient in aminoacyl-tRNA synthetase genes, when tetracycline was added, the content of plasmid per the culture solution was maintained, but when tetracycline was not added, the content of plasmid was observed to drop sharply. Further, plasmid pDAC1 was prepared in which a cellulase gene having pDATSC1 was inserted into the BamHI recognition site of pDA2 and the same tests were conducted. This revealed that in the mutant strain of Bacillus subtilis strain ISW1214 having pDAC1, when tetracycline was added, the plasmid content per the culture solution was maintained, but when it was not added, the plasmid content was observed to drop sharply. To facilitate comprehension of these results, FIG. 1 shows the results of electrophoresis of solutions of the plasmids recovered from the various cells cultured in a medium to which no antibiotic was added. Following the electrophoresis operation, the agarose gel was immersed in ethidium bromide to dye the DNA. In FIG. 1, lane 1 is the transformation combination of a host cell deficient in the particular aminoacyl-tRNA synthetase gene on the chromosome but containing the aminoacyl-tRNA synthetase gene in a plasmid; lane 2 is the transformation combination of a host cell non deficient in the particular aminoacyl-tRNA synthetase gene on the chromosome and having the aminoacyl-tRNA synthetase gene in a plasmid; and lane 3 is the transformation combination of a host cell not deficient in the particular aminoacyl-tRNA synthetase gene on the chromosome but not containing the aminoacyl-tRNA synthetase gene in a plasmid.

### [Embodiment 7] Production of Agarase in Strain DTS1451

A PCR reaction was conducted using primers W and X with the chromosome of Microbulbifer sp. A94, which is an agarase-producing bacterium (see Biosci Biotechnol Biochem. (2004) 68: 1073-81. Ohta, Y. et al., the entire description of which is hereby incorporated in its entirety) as template. A DNA fragment encoding agarase was obtained. Additionally, a PCR reaction was conducted using primers Y and Z with above-described pDATSC1 as template. The 5′ end of the DNA fragment obtained was phosphorylated, after which the fragment was spliced to DNA encoding agarase. This was then used to transform Escherichia coli HB101. A plasmid prepared from a transformant obtained that had agarase activity was named pDATSA1. This plasmid was used to transform DTS1451(pDATSK). Selection was conducted with regenerated medium containing 7.5 microgram/mL of tetracycline. The transformant obtained was inoculated into LB agar medium containing kanamycin and regenerated medium containing 7.5 micrograms/mL of tetracycline. A strain exhibiting agarase activity, sensitivity to kanamycin, and resistance to tetracycline was named DTS1451(pDATSA1). DTS1451(pDATSA1) was cultured with stirring at 130 rpm for 72 hours at 30° C. in PPS medium (to which no antibiotic was added) and the agarase activity of the supernatant of the culture obtained was measured. As a result, the production of about 0.1 g of agarase per liter of culture solution was confirmed. Next, a PCR reaction was conducted using primer A1 (SEQ ID NO: 79) and primer B1 (SEQ ID NO: 80) with pDATSA1 as template. The DNA fragment obtained was digested with XhoI and closed into a circle by a ligation reaction. This operation yielded a plasmid from which the tryptophanyl-tRNA synthetase gene had been eliminated. This plasmid was cleaved with the restriction enzyme EcoRI. Additionally, DNA fragments containing the tryptophanyl-tRNA synthetase gene and nearby regions were amplified by PCR in combinations of primers C1 (SEQ ID NO: 81) and D1 (SEQ ID NO: 82), primers E1 (SEQ ID NO: 83) and F1 (SEQ ID NO: 84), or primers G1 (SEQ ID NO: 85) and H1 (SEQ ID NO: 86), with pDATSC1 as template. The various PCR-amplified DNA fragments were cleaved with the reduction enzyme EcoRI and spliced with the above plasmid that had been digested in advance with the restriction enzyme EcoRI. These were then used to transform Escherichia coli HB101. Plasmids prepared from those transformants exhibiting agarase activity were named pDATSA2, pDATSA3, and pDATSA4. The base sequences of pDATSA2, pDATSA3, and pDATSA4 were determined, revealing that the tryptophanyl-tRNA synthetase gene and the surrounding portions had the sequences indicated in SEQ ID NOS: 87, 88, and 89. Next, these plasmids were used to transform DTS1451(pDATSK) and selection was conducted with regenerated medium containing 7.5 micrograms/mL of tetracycline. The transformants were inoculated into LB agar medium containing kanamycin and regenerated medium containing 7.5 microgram/mL of tetracycline. Those strains exhibiting agarase activity, sensitivity to kanamycin, and resistance to tetracycline were selected. The DTS1451(pDATSA2), DTS1451(pDATSA3), and DTS1451(pDATSA4) obtained were cultured with stirring at 130 rpm for 72 hours at 30° C. in PPS medium (to which no antibiotic was added) and the agarase activity of the supernatant of the cultures obtained was measured. The results confirmed an agarase production of about 0.1 g per liter of culture solution for each of the three.

TABLE 1

| | | SEQ |
|---|---|---|
| | | ID |
| Primer | Nulceic acid sequence | NO |
| A | GTGCCTCGAGGATTAAGCATTGGTAACTGTC | 53 |
| B | TATTCTCGAGACATTAACCTAGAAAGCACTAAGG | 54 |
| C | CTGCCTGTCTCGAGGCTGATAGCAGTTATC | 55 |
| D | CAACTCGAGGCTGGTCGGACAAACACCTAG | 56 |
| E | TGCGAATTCCGGATGTAAGGAGAACGGCTC | 57 |
| F | CTTGTCGACGTACATCAGAAATCGTACATTCG | 58 |
| G | GGTGGATCCCCTCGCCGGCAATAGTTACCC | 59 |
| H | AATCTAGACTTTCGTTATACAAATTTTAACC | 60 |
| I | TGTAGGATCCAATATGGGCAGAAAACACATG | 61 |
| J | ATATCTAGATTCCACTCTGTGTACCAGTAGC | 62 |
| K | GTTAGCTCCTTCGGTCCTCC | 63 |
| L | GTAACTGGCTTCAGCAGAGC | 64 |
| M | CTGCCACATCGTCTAGGCTGC | 65 |
| N | CAGGAGTCCAAATACCAGAG | 66 |
| O | CCCTTGCCTATACTGTGGAC | 67 |
| P | GTTACAATAGCGACGGAGAG | 68 |
| Q | GTCTGAATTCGAGGAAGGTTTACACCG | 69 |
| R | CTGCGAATTCATCTTCATGGTGAACC | 70 |
| S | CATTGTCATTAGTTGGCTGG | 71 |
| T | GCAAGAATTCAAAATCCATCTTCATCGG | 72 |
| U | GTGACTGAGGATCCGCTAGTTCCAGATCG | 73 |
| V | CTCTTGGATCCCTTCATCATTCTATCACAC | 74 |
| W | GCAGATTGGGATGGAGTTCCCGTAC | 75 |
| X | TTACAGCTTCACAAAGCGGATTTC | 76 |
| Y | TGCTGCAAGAGCTGTCGGAAATAAAG | 77 |
| Z | AAGAAGAAAAGAAAGAAGCTAAAG | 78 |

### INDUSTRIAL APPLICABILITY

The present invention is useful in the field of producing various proteins and peptides by culturing transformants obtained by transforming host cells such as bacteria.

### BRIEF DESCRIPTION OF THE DRAWINGS

FIG. 1 shows the results of electrophoresis of a plasmid recovery solution.

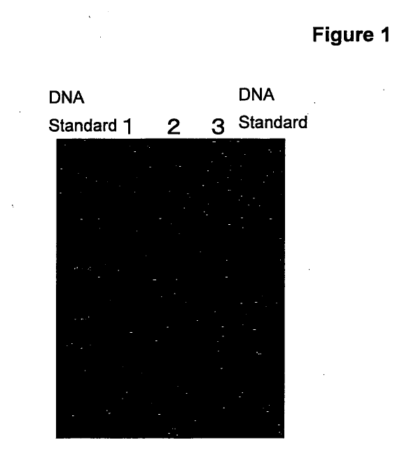

1. A recombinant vector, comprising a site permitting insertion in an expressible state of a foreign gene other than a gene encoding an aminoacyl-tRNA synthetase and comprising a gene encoding aminoacyl-tRNA synthetase in an expressible state, for use in a mutant host in which a chromosomal gene encoding an aminoacyl-tRNA synthetase has been knocked out, or in a mutant host in which expression of a chromosomal gene encoding an aminoacyl-tRNA synthetase has been diminished to a degree to which the host cell cannot grow.

2. The recombinant vector according to claim 1, wherein said aminoacyl-tRNA synthetase is tryptophanyl-tRNA synthetase, alanyl-tRNA synthetase, arginyl-tRNA synthetase, asparginyl-tRNA synthetase, aspartyl-tRNA synthetase, cysteinyl-tRNA synthetase, glutamine-tRNA synthetase, glutamate-tRNA synthetase, glycine-tRNA synthetase, histidyl-tRNA synthetase, isoleucyl-tRNA synthetase, leucyl-tRNA synthetase, lysine-tRNA synthetase, methionyl-tRNA synthetase, phenylalanine-tRNA synthetase, prolyl-tRNA synthetase, seryl-tRNA synthetase, threonyl-tRNA synthetase, tyrosyl-tRNA synthetase, or valyl-tRNA synthetase.

3. The recombinant vector according to claim 1, wherein said recombinant vector is in the form of a plasmid, bacteriophage, or retrotransposon.

4. A mutant host cell in which a chromosomal gene encoding an aminoacyl-tRNA synthetase has been knocked out, or a mutant host in which the expression of a chromosomal gene encoding an aminoacyl-tRNA synthetase has been diminished to a degree where the host cell cannot grow, that is used for transformation by a recombinant vector comprising a site permitting insertion in an expressible state of a foreign gene other than a gene encoding an aminoacyl-tRNA synthetase and comprising in an expressible state a gene encoding an aminoacyl-tRNA synthetase.

5. The mutant host cell according to claim 4, wherein said aminoacyl-tRNA synthetase is tryptophanyl-tRNA synthetase, alanyl-tRNA synthetase, arginyl-tRNA synthetase, asparginyl-tRNA synthetase, aspartyl-tRNA synthetase, cysteinyl-tRNA synthetase, glutamine-tRNA synthetase, glutamate-tRNA synthetase, glycine-tRNA synthetase, histidyl-tRNA synthetase, isoleucyl-tRNA synthetase, leucyl-tRNA synthetase, lysine-tRNA synthetase, methionyl-tRNA synthetase, phenylalanine-tRNA synthetase, prolyl-tRNA synthetase, seryl-tRNA synthetase, threonyl-tRNA synthetase, tyrosyl-tRNA synthetase, or valyl-tRNA synthetase.

6. The mutant host cell according to claim 4, wherein the host cell into which a mutation is introduced is a bacterium, yeast, animal cell, or plant cell.

7. The mutant host cell according to claim 4, wherein the host cell into which a mutation is introduced is a bacterium or yeast.

8. The mutant host cell according to claim 6, wherein the bacterium is a bacterium of the genus Bacillus.

9. A method of preparing a protein or peptide encoded by a foreign gene by expressing a foreign gene other than a gene encoding an aminoacyl-tRNA synthetase, comprising:
a step of preparing a recombinant vector, comprising a site permitting insertion in an expressible state of a foreign gene and comprising in an expressible state a gene encoding an aminoacyl-tRNA synthetase, in which a desired foreign gene has been inserted at the site permitting insertion in an expressible state of a foreign gene;
a step of preparing a mutant host cell in which a chromosomal gene encoding an aminoacyl-tRNA synthetase has been knocked out or a mutant host cell in which the expression of a chromosomal gene encoding an aminoacyl-tRNA synthetase has been diminished to a degree where the host cell cannot grow;
a step of transforming said mutant host cell with said recombinant vector to obtain a transformant; and
a step of culturing said transformant to prepare the protein or peptide encoded by the foreign gene.

10. A method of causing a targeted recombinant vector to be stably retained within a host cell, comprising:
knocking out, or diminishing to a degree where the host cell cannot grow, a chromosomal gene encoding an aminoacyl-tRNA synthetase contained in the host cell, to convert the host cell to a mutant host cell;
incorporating in an expressible state a gene encoding an aminoacyl-tRNA synthetase into the targeted recombinant vector; and
transforming the mutant host cell with said targeted recombinant vector containing the gene encoding an aminoacyl-tRNA synthetase in an expressible state.

11. The method according to claim 10, wherein the recombinant vector comprises a site permitting insertion in an expressible state of a foreign gene other than a gene encoding an aminoacyl-tRNA synthetase.

12. The method according to claim 9, wherein said aminoacyl-tRNA synthetase is tryptophanyl-tRNA synthetase, alanyl-tRNA synthetase, arginyl-tRNA synthetase, asparginyl-tRNA synthetase, aspartyl-tRNA synthetase, cysteinyl-tRNA synthetase, glutamine-tRNA synthetase, glutamate-tRNA synthetase, glycine-tRNA synthetase, histidyl-tRNA synthetase, isoleucyl-tRNA synthetase, leucyl-tRNA synthetase, lysine-tRNA synthetase, methionyl-tRNA synthetase, phenylalanine-tRNA synthetase, prolyl-tRNA synthetase, seryl-tRNA synthetase, threonyl-tRNA synthetase, tyrosyl-tRNA synthetase, or valyl-tRNA synthetase.

13. The method according to claim 9, wherein said host cell into which a mutation is incorporated is a bacterium, yeast, animal cell, or plant cell.

14. The method according to claim 9, wherein said host cell into which a mutation is incorporated is a bacterium or yeast.

15. The method according to claim 13, wherein said bacterium is a bacterium of the genus Bacillus.

16. The method according to claim 9, wherein the protein encoded by the foreign gene is an enzyme selected from the group consisting of oxidoreductases, transferases, hydrolases, phosphorylases, lyases, isomerases, ligases/synthetases, and modifying enzymes.


SEQUENCE LISTING (89 sequences)

SEQ ID NO: 1 - DNA, 2631 bp, E. coli
  alaS Alanine-tRNA synthetase
atgagcaagagcaccgctgagatccgtcaggcgtttctcgactttttccatagtaagggacatcaggtagttgccagcagctccctggtaccccataacgacccaactttgttgtttaccaacgccgggatgaaccagttcaaggatgtgttccttgggctcgacaagcgtaattattcccgcgctaccacttcccaacgctgcgtgcgtgcgggtggtaaacacaacgacctggaaaacgtcggttacaccgcgcgtcaccataccttcttcgaaatgctgggcaacttcagcttcggcgactatttcaaacacgatgccattcagtttgcatgggaactgctgaccagcgaaaaatggtttgccctgccgaaagagcgtctgtgggttaccgtctatgaaagcgacgacgaagcctacgaaatctgggaaaaagaagtagggatcccgcgcgaacgtattattcgcatcggcgataacaaaggtgcgccatacgcatctgacaacttctggcagatgggtgacactggtccgtgcggcccgtgcaccgaaatcttctacgatcacggcgaccacatttgggggggccctccgggaagcccggaagaagacggcgaccgctacattgagatctggaacatcgtcttcatgcagttcaaccgccaggccgatggcacgatggaaccgctgccgaagccgtctgtagataccggtatgggtctggagcgtattgctgcggtgctgcaacacgttaactctaactatgacatcgacctgttccgcacgctgatccaggcggtagcgaaagtcactggcgcaaccgatctgagcaataaatcgctgcgcgtaatcgctgaccacattcgttcttgtgcgttcctgatcgcggatggcgtaatgccgtccaatgaaaaccgtggttatgtactgcgtcgtatcattcgtcgcgcagtgcgtcacggtaatatgctcggcgcgaaagaaaccttcttctacaaactggttggtccgctgatcgacgttatgggctctgcgggtgaagacctgaaacgccagcaggcgcaggttgagcaggtgctgaagactgaagaagagcagtttgctcgtactctggagcgcggtctggcgttgctggatgaagagctggcaaaactttctggtgatacgctggatggtgaaactgctttccgtctgtacgacacctatggcttcccggttgacctgacggctgatgtttgtcgtgagcgcaacatcaaagttgacgaagctggttttgaagctgcaatggaagagcagcgtcgtcgcgcgcgcgaagccagcggctttggtgccgattacaacgcaatgatccgtgttgacagtgcatctgaatttaaaggctatgaccatctggaactgaacggcaaagtgactgcgctgtttgttgatggtaaagcggttgatgccatcaatgcaggccaggaagctgtggtcgtgctggatcaaacgccattctatgcggaatccggcggtcaggttggcgataaaggcgaactgaaaggcgctaacttctcctttgcggtggaagatacgcagaaatacggccaggcgattggtcacatcggtaaacttgctgcgggttctctgaaagtgggcgacgcggtgcaggctgatgttgatgaggctcgtcgcgcccgtattcgtctgaatcactccgcaacgcacctgatgcacgctgcgctgcgccaggttctgggtactcatgtatcgcagaaaggttcactggttaacgacaaggtgctgcgcttcgacttctcacacaacgaagcgatgaaaccagaagagattcgtgcggtcgaagacctggtgaacacacagattcgtcgcaatttgccgatcgaaaccaacatcatggatctcgaagcggcgaaagcgaaaggtgcgatggcgctgttcggcgagaagtatgatgagcgcgtacgcgtgctgagcatgggcgatttctctaccgagttgtgtggcggtactcacgccagccgcactggtgatattggtctgttccgcatcatctctgaatcgggtactgctgcaggcgttcgtcgtatcgaagcggtaaccggagaaggtgctatcgccaccgttcatgcagacagcgatcgcttaagcgaagtcgcgcatctgctgaaaggcgatagcaataatctggctgataaagtgcgctcagtactggaacgtacgcgtcagctggaaaaagagttacaacagcttaaagaacaagctgccgcacaggagagcgcaaatctttccagtaaggcaattgatgttaatggtgttaagctgttggttagcgagcttagcggtgttgagccgaaaatgttgcgtaccatggttgacgatttaaaaaatcagctggggtcgacaattatcgtgctggcaacggtagtcgaaggtaaggtttctctgattgcaggcgtatctaaggacgtcacagatcgtgtgaaagcaggggaactgattggtatggtcgctcagcaggtgggcggcaagggtggtggacgtcctgacatggcgcaagccggtggtacggatgctgcggccttacctgcagcgttagccagtgtgaaaggctgggtcagcgcgaaattgcaataa

SEQ ID NO: 2 - DNA, 1734 bp, E. coli
  argS Arginine-tRNA synthetase
gtgaatattcaggctcttctctcagaaaaagtccgtcaggccatgattgcggcaggcgcgcctgcggattgcgaaccgcaggttcgtcagtcagcaaaagttcagttcggcgactatcaggctaacggcatgatggcagttgctaaaaaactgggtatggcaccgcgacaattagcagagcaggtgctgactcatctggatcttaacggtatcgccagcaaagttgagatcgccggtccaggctttatcaacattttccttgatccggcattcctggctgaacatgttcagcaggcgctggcgtccgatcgtctcggtgttgctacgccagaaaaacagaccattgtggttgactactctgcgccaaacgtggcgaaagagatgcatgtcggtcacctgcgctctaccattattggtgacgcagcagtgcgtactctggagttcctcggtcacaaagtgattcgcgcaaaccacgtcggcgactggggcactcagttcggtatgctgattgcatggctggaaaagcagcagcaggaaaacgccggtgaaatggagctggctgaccttgaaggtttctaccgcgatgcgaaaaagcattacgatgaagatgaagagttcgccgagcgcgcacgtaactacgtggtaaaactgcaaagcggtgacgaatatttccgcgagatgtggcgcaaactggtcgacatcaccatgacgcagaaccagatcacctacgatcgtctcaacgtgacgctgacccgtgatgacgtgatgggcgaaagcctctacaacccgatgctgccaggaattgtggcggatctcaaagccaaaggtctggcagtagaaagcgaaggggcgaccgtcgtattccttgatgagtttaaaaacaaggaaggcgaaccgatgggcgtgatcattcagaagaaagatggcggctatctctacaccaccactgatatcgcctgtgcgaaatatcgttatgaaacactgcatgccgatcgcgtgctgtattacatcgactcccgtcagcatcaacacctgatgcaggcatgggcgatcgtccgtaaagcaggctatgtaccggaatccgtaccgctggaacaccacatgttcggcatgatgctgggtaaagacggcaaaccgttcaaaacccgcgcgggtggtacagtgaaactggccgatctgctggatgaagccctggaacgtgcacgccgtctggtggcagaaaagaacccggatatgccagccgacgagctggaaaaactggctaacgcggttggtattggtgcggtgaaatatgcggatctctccaaaaaccgcaccacggactacatcttcgactgggacaacatgctggcgtttgagggtaataccgcgccatacatgcagtatgcatacacgcgtgtattgtccgtgttccgtaaagcagaaattgacgaagagcaactggctgcagctccggttatcatccgtgaagatcgtgaagcgcaactggcagctcgcctgctgcagtttgaagaaaccctcaccgtggttgcccgtgaaggcacgccgcatgtaatgtgtgcttacctgtacgatctggccggtctgttctctggcttctacgagcactgcccgatcctcagcgcagaaaacgaagaagtgcgtaacagccgtctaaaactggcacaactgacggcgaagacgctgaagctgggtctggatacgctgggtattgagactgtagagcgtatgtaa

SEQ ID NO: 3 - DNA, 1401 bp, E. coli
  asnS Asparagine-tRNA synthetase
atgagcgttgtgcctgtagccgacgtactccagggccgtgtagccgttgacagcgaagtcaccgtgcgcggatgggtacgtacccgccgagattcaaaagctggcatctccttcctcgccgtttatgacggttcctgctttgatcctgtacaggctgtcatcaataattctctgcccaattacaatgaagacgtcctgcgtctgaccaccggctgctcggtcattgtgacgggtaaagtcgtggcgtcgccgggccaggggcaacaatttgaaattcaggccagcaaggttgaagttgctggttgggttgaagatccagacacttacccgatggcggcaaaacgccacagcattgagtatctgcgtgaagtcgctcacctgcgtccgcgcacaaacctgattggtgccgtcgcgcgcgttcgccatacgctggcgcaggcgctgcatcgcttctttaacgagcagggattcttctgggtttcaacgccactgattaccgcatctgataccgaaggtgcaggcgaaatgttccgcgtttctacgctggatctggaaaacctgccgcgtaacgatcagggcaaagtggatttcgacaaagacttctttggtaaagagtctttcctgaccgtatctggccagttgaacggcgaaacctacgcttgcgcattgtccaaaatttataccttcggcccgactttccgtgctgaaaactccaacaccagccgtcacctggcggaattctggatgctggagccggaagtggcgtttgctaacctgaacgatattgcgggtctggctgaagccatgctgaaatatgtcttcaaagcggttctcgaagaacgcgctgacgacatgaaattcttcgctgaacgcgtagataaagatgccgtttcacgtctggaacgcttcattgaagccgattttgcgcaggtggattataccgacgcagtgaccattctcgaaaactgcggcaggaagtttgaaaacccggtttactggggagtcgatctctcttctgagcatgagcgttatctggcggaagaacactttaaagcaccggtagtggttaaaaactatccgaaagatattaaagcgttctatatgcgccttaacgaagacggtaaaaccgttgcggctatggacgttctggctccgggcatcggtgagatcattggtggctcccagcgtgaagaacgtctggacgtgctggacgagcgtatgctggaaatgggcctgaataaagaagattactggtggtatcgcgatctgcgtcgctacggtactgttccgcattcaggtttcggtcttggttttgaacgtctgattgcttacgtaactggcgtgcaaaacgtacgtgatgtgattccgttcccacgtactccgcgtaacgccagcttctaa

SEQ ID NO: 4 - DNA, 1773 bp, E. coli
  aspS Aspartate-tRNA synthetase
atgcgtacagaatattgtggacagctccgtttgtcccacgtggggcagcaggtgactctgtgtggttgggtcaaccgtcgtcgtgatcttggtagcctgatcttcatcgatatgcgcgaccgcgaaggtatcgtgcaggtatttttcgatccggatcgtgcggacgcgttaaagctggcctctgaactgcgtaatgagttctgcattcaggtcacgggcaccgtacgtgcgcgtgacgaaaaaaatattaaccgcgatatggcgaccggcgaaatcgaagtgctggcgtcctcgctgactatcatcaaccgcgcagatgttctgccgcttgactctaaccacgtcaacaccgaagaagcgcgtctgaaataccgctacctcgacctgcgtcgtccggaaatggctcagcgcctgaaaacccgcgctaaaatcaccagcctggtgcgccgttttatggatgaccacggcttcctcgacatcgaaactccgatgctgaccaaagccacgccggaaggcgcgcgtgactacctggtgccttctcgtgtgcacaaaggtaaattctacgcactgccgcaatccccgcagttgttcaaacagctgctgatgatgtccggttttgaccgttactatcagatcgttaaatgcttccgtgacgaagacctgcgtgctgaccgtcagcctgaatttactcagatcgatgtggaaacttctttcatgaccgcgccgcaagtgcgtgaagtgatggaagcgctggtgcgtcatctgtggctggaagtgaagggtgtggatctgggcgatttcccggtaatgacctttgcggaagcagaacgccgttatggttctgataaaccggatctgcgtaacccgatggaactgactgacgttgctgatctgctgaaatctgttgagtttgctgtatttgcaggtccggcgaacgatccgaaaggtcgcgtagcggctctgcgcgttccgggcggcgcatcgctgacccgtaagcagatcgacgaatacggtaacttcgttaaaatctacggcgcgaaaggtctggcttacatcaaagttaacgaacgcgcgaaaggtctggaaggtatcaacagcccggtagcgaagttccttaatgcagaaatcatcgaagacatcctggatcgtactgccgcgcaagatggcgatatgattttcttcggtgccgacaacaagaaaattgttgccgacgcgatgggtgcactgcgcctgaaagtgggtaaagaccttggtctgaccgacgaaagcaaatgggcaccgctgtgggttatcgacttcccgatgtttgaagacgacggtgaaggcggcctgacggcaatgcaccatccgttcacctcaccgaaagatatgacggctgcagaactgaaagctgcaccggaaaatgcggtggcgaacgcttacgatatggtcatcaatggttacgaagtgggcggtggttcagtacgtatccataatggtgatatgcagcagacggtgtttggtattctgggtatcaacgaagaggaacagcgcgagaaattcggcttcctgctcgacgctctgaaatacggtactccgccgcacgcaggtctggcattcggtcttgaccgtctgaccatgctgctgaccggcaccgacaatatccgtgacgttatcgccttcccgaaaaccacggcggcagcgtgtctgatgactgaagcaccgagctttgctaacccgactgcactggctgagctgagcattcaggttgtgaagaaggctgagaataactga

SEQ ID NO: 5 - DNA, 1386 bp, E. coli
  cysS Cysteine-tRNA synthetase
atgctaaaaatcttcaatactctgacacgccaaaaagaggaatttaagcctattcacgccggggaagtcggcatgtacgtgtgtggaatcaccgtttacgatctctgtcatatcggtcacgggcgtacctttgttgcttttgacgtggttgcgcgctatctgcgtttcctcggctataaactgaagtatgtgcgcaacattaccgatatcgacgacaaaatcatcaaacgcgccaatgaaaatggcgaaagctttgtggcgatggtggatcgcatgatcgccgaaatgcacaaagattttgatgctttgaacattctgcgcccggatatggagccgcgcgcgacgcaccatatcgcagaaattattgaactcactgaacaactgatcgccaaaggtcacgcttatgtggcggacaacggcgacgtgatgttcgacgtcccgaccgatccaacttatggcgtgctgtcgcgtcaggatctcgaccagctgcaggcaggcgcgcgcgttgacgtggtcgacgacaaacgcaacccaatggacttcgttctgtggaagatgtcgaaagagggcgaaccgagctggccgtctccgtggggcgcgggtcgtcctggctggcacattgaatgttcggcaatgaactgcaagcagctgggtaaccactttgatatccacggcggcggttcagacctgatgttcccgcaccacgaaaacgaaatcgcgcagtccacctgtgcccatgatggtcagtatgtgaactactggatgcactcggggatggtgatggttgaccgcgagaagatgtccaaatcgctgggtaacttctttaccgtgcgcgatgtgctgaaatactacgacgcggaaaccgtgcgttacttcctgatgtcgggccactatcgcagccagttgaactacagcgaagagaacctgaagcaggcgcgtgcggcgctggagcgtctctacactgcgctgcgcggcacagataaaaccgttgcgcctgccggtggcgaagcgtttgaagcgcgctttattgaagcgatggacgacgatttcaacaccccggaagcctattccgtactgtttgatatggcgcgtgaagtaaaccgtctgaaagcagaagatatggcagcggcgaatgcaatggcatctcacctgcgtaaactttccgctgtattgggcctgctggagcaagaaccggaagcgttcctgcaaagcggcgcgcaggcagacgacagcgaagtggctgagattgaagcgttaattcaacagcgtctggatgcccgtaaagcgaaagactgggcggcggcggatgcggcgcgtgatcgtcttaacgagatggggatcgtgctggaagatggcccgcaagggaccacctggcgtcgtaagtaa

SEQ ID NO: 6 - DNA, 1665 bp, E. coli
  glnS Glutamine-tRNA synthetase
atgagtgaggcagaagcccgcccgactaactttatccgtcagatcatcgatgaagatctggccagtggtaagcacaccacagtacacacccgtttcccgccggagccgaatggctatctgcatattggccatgcgaaatctatctgcctgaacttcgggatcgcccaggactataaaggccagtgcaacctgcgtttcgacgacactaacccggtaaaagaagatatcgagtatgttgagtcgatcaaaaacgacgtagagtggttaggttttcactggtctggtaacgtccgttactcctccgattattttgatcagctccacgcctatgcgatcgaactgatcaataaaggcctggcgtacgttgatgaactgacgccggaacagatccgcgaataccgcggcaccctgacgcaaccgggtaaaaacagcccgtaccgcgaccgcagcgttgaagagaacctggcgctgttcgaaaaaatgcgtgccggtggttttgaagaaggtaaagcctgcctgcgtgcgaaaatcgacatggcttcaccgtttatcgtgatgcgcgatccggtgctgtaccgtattaaatttgctgaacaccaccagactggcaacaagtggtgcatctacccgatgtacgacttcacccactgcatcagcgatgccctggaaggtattacgcactctctgtgtacgcttgagttccaggacaaccgtcgtctgtacgactgggtactggacaacatcacgattcctgttcacccgcgccagtatgagttctcgcgcctgaatctggaatacaccgtgatgtccaagcgtaagttgaacctgctggtgaccgacaagcacgttgaaggctgggatgacccgcgtatgccgaccatttccggtctgcgtcgtcgtggttacactgcggcttctattcgtgagttctgcaaacgcatcggcgtgaccaagcaggacaacaccattgagatggcgtcgctggaatcctgcatccgtgaagatctcaacgaaaatgcgccgcgcgcaatggcggttatcgatccggtgaaactggttatcgaaaactatcagggcgaaggcgaaatggttaccatgccgaaccatccgaacaaaccggaaatgggcagccgtcaggtgccgtttagcggtgagatttggattgatcgcgccgatttccgcgaagaagctaacaagcagtacaaacgtctggtgctgggtaaagaagtgcgtctgcgtaatgcttatgtgattaaggcagaacgcgtcgagaaagatgccgaaggtaatatcaccaccatcttctgtacttatgacgccgataccttaagcaaagatccggcagatggtcgtaaagtcaaaggtgttattcactgggtgagcgcggcacatgcgctgccggttgaaatccgtttgtatgaccgtctgttcagcgtgcctaacccaggtgctgcggatgatttcctgtcggtgattaacccggaatcgctggtgatcaaacagggctttgctgaaccgtcgctgaaagatgcggttgcgggtaaagcattccagtttgagcgtgaaggttacttctgcctcgatagccgccattctacggcggaaaaaccggtatttaaccgcaccgttgggctgcgtgatacctgggcgaaagtaggcgagtaa

SEQ ID NO: 7 - DNA, 1665 bp, E. coli; identical to SEQ ID NO: 6
  gltX Glutamate-tRNA synthetase

SEQ ID NO: 8 - DNA, 912 bp, E. coli
  glyQ Glycine-tRNA synthetase, alpha-subunit
atgcaaaagtttgataccaggaccttccagggcttgatcctgaccttacaggattactgggctcgccagggctgcaccattgttcaaccattggacatggaagtcggcgcgggaacctctcacccaatgacctgtctgcgcgagctggggccagaaccgatggcggctgcttatgttcagccttctcgtcgcccgaccgatggtcgctacggcgaaaaccccaaccgtttacagcactactatcagttccaggtggtcattaagccatcgccggacaatattcaggagctgtacctcggttctctgaaagagctgggcatggacccgactattcacgacatccgtttcgtggaagataactgggaaaacccgacgctgggtgcctggggactgggctgggaagtgtggctgaacggcatggaagtgacgcagttcacttacttccagcaggttggtggtctggagtgtaaaccggttaccggcgagatcacctacggtctggaacgtctggccatgtacattcagggcgtagacagcgtttacgacctggtctggagcgacggcccgctgggtaaaaccacctacggcgacgtgttccatcagaacgaagtggagcagtccacttacaacttcgaatacgcggatgtggacttcctgttcacctgcttcgagcagtacgagaaagaagcgcagcagctgctggcgctggaaaatccgctgccgctgccagcctacgagcgtattctgaaagccgcccacagcttcaacctgctggatgcgcgtaaagccatctccgtcaccgagcgtcagcgctacattctgcgcattcgcaccctgaccaaagcagtggcagaagcatactacgcttcccgtgaagccctcggcttcccgatgtgcaacaaagataagtaa

SEQ ID NO: 9 - DNA, 2070 bp, E. coli
  glyS Glycine-tRNA synthetase, beta-subunit
atgtctgagaaaacttttctggtggaaatcggcactgaagagctgccaccaaaagcactgcgcagcctggctgagtcctttgctgcgaactttactgcggagctggataacgctggcctcgcacacggcaccgttcaatggtttgctgctccgcgtcgtctggcgctgaaagtagctaacctggcggaagcgcaaccggatcgtgaaatcgaaaaacgcggcccggcgattgcccaggcgttcgacgctgaaggcaaaccgagcaaagcggcagaaggttgggcgcgtggttgcggtattaccgttgaccaggctgagcgtctgactaccgataaaggcgaatggctgctgtatcgcgcccatgtgaagggcgaaagcaccgaagcactgctgccgaatatggttgcgacttctctggcgaaactgccgatcccgaaactgatgcgttggggcgcaagcgacgtgcacttcgtgcgtccggtgcacaccgtgaccctgctgctgggcgacaaagtcattccggcaaccattctgggcattcagtccgatcgcgtgattcgcggccaccgctttatgggcgagccggaattcaccatcgataacgccgatcagtatccggaaattctgcgtgagcgtgggaaagtcatcgccgattacgaagaacgtaaggcgaagattaaagccgatgccgaagaagcagcgcgtaagattggcggtaacgctgacttaagcgaaagcctgctggaagaagtggcttcgctggtggagtggccggtcgttctgaccgcaaaattcgaagagaaattcctcgcggtgccggctgaagcgctggtttacaccatgaaaggtgaccagaaatacttcccggtgtatgcgaacgacggcaaactgctgccgaactttatcttcgttgccaacatcgaatcgaaagatccgcagcagattatctccggtaacgagaaagtcgttcgtccgcgtctggcggatgccgagttcttcttcaacaccgaccgtaaaaaacgtcttgaagataacctgccgcgcctgcaaaccgtgttgttccagcaacagttggggacgctgcgcgacaaaactgaccgcatccaggcgctggctggctggattgctgaacagattggcgctgacgttaaccacgctacccgtgcgggtctgctgtctaagtgcgacctgatgaccaacatggtcttcgagttcaccgacacccagggcgttatggggatgcactatgcgcgtcacgatggcgaagcggaagatgtcgcggtggcgctgaatgagcagtatcagccgcgttttgctggtgatgacctgccgtccaacccagtagcttgtgcgctggcgattgctgacaagatggataccctggcgggtatcttcggtatcggtcagcatccgaaaggcgacaaagacccgtttgcgctgcgtcgtgccgcgcttggcgtgctgcgaattatcgttgagaagaacctcaaccttgatctgcaaacgctgaccgaagaagcggtgcgtctgtatggcgataagctgactaatgccaacgtagttgatgatgttatcgactttatgctcggtcgcttccgcgcctggtatcaggacgaaggttataccgttgacaccatccaggcggtactggcgcgtcgtccgactcgtccggctgatttcgatgcccgtatgaaagcggtatcgcatttccgtaccctggatgcagctgctgcactggcggcggcgaacaaacgtgtatctaacattctggcgaaatctgacgaagtgctgagcgaccgcgtgaatgcctctaccctgaaagagccggaagaaattaaactggcgatgcaggttgtggtgctacgtgacaagctggagccgtactttacggaaggtcgttaccaggatgcgctggtcgaactggctgagctgcgtgaaccggttgatgctttcttcgataaagtgatggtcatggttgatgacaaagaattgcgtatcaaccgtctgaccatgctggagaaactgcgcgaactgttcctgcgcgttgcggatatttcgctgttgcaataa

SEQ ID NO: 10 - DNA, 1275 bp, E. coli
  hisS Histidine-tRNA synthetase
gtggcaaaaaacattcaagccattcgcggcatgaacgattacctgcctggcgaaacggccatctggcagcgcattgaaggcacactgaaaaacgtgctcggcagctacggttacagtgaaatccgcttgccgattgtagagcagaccccgctattcaaacgtgcgattggtgaagtcaccgacgtggttgaaaaagagatgtacacctttgaggatcgcaatggcgacagcctgactctgcgccctgaagggacggcgggctgtgtacgcgccggcatcgagcatggtcttctgtacaatcaggaacagcgtctgtggtatatcgggccgatgttccgtcacgagcgtccgcagaaagggcgttatcgtcagttccatcagttgggctgcgaagttttcggtctgcaaggtccggatatcgacgctgaactgattatgctcactgcccgctggtggcgcgcgctgggtatttccgagcacgtaactcttgagctgaactctatcggttcgctggaagcacgcgccaattaccgcgatgcgctggtggcattccttgagcagcataaagaaaagctggacgaagactgcaaacgccgcatgtacactaacccgctgcgcgtgctggattcaaaaaatccggaagtgcaggcgcttctcaacgacgctccggcattaggtgactatctggacgaggaatctcgtgagcattttgccggtctgtgcaaactgctggagagcgcggggatcgcttacaccgtaaaccagcgtctggtgcgtggtctggattactacaaccgtaccgttttcgagtgggtgactaacagtctcggctcccagggcaccgtgtgtgcaggcggtcgttatgacggtcttgtggaacaactgggcggtcgtgcaacaccggctgtcggttttgctatgggcctcgaacgtcttgtattgttagtacaggccgttaatccggaatttaaagccgatcctgttgtcgatatatacctggtggcttcaggtgctgatacacaatctgcggctatggcattagctgagcgtctgcgtgatgaattaccgggcgtgaaattgatgaccaaccacggcggcggcaactttaagaaacagtttgcccgtgctgataaatggggtgcccgcgttgctgtggtgctgggtgagtctgaagtggctaacggcacagcagtagtgaaggatttgcgctctggtgagcaaacggcagttgcgcaggatagcgtagccgcgcatttgcgcacgttactgggttaa

SEQ ID NO: 11 - DNA, 2817 bp, E. coli
  ileS Isoleucine-tRNA synthetase
atgagtgactataaatcaaccctgaatttgccggaaacagggttcccgatgcgtggcgatctcgccaagcgcgaacccggaatgctggcgcgttggactgatgatgatctgtacggcatcatccgtgcggctaaaaaaggcaaaaaaaccttcattctgcatgatggccctccttatgcgaatggcagcattcatattggtcactcggttaacaagattctgaaagacattatcgtgaagtccaaagggctttccggttatgactcgccgtatgtgcctggctgggactgccacggtctgccgatcgagctgaaagtcgagcaagaatacggtaagccgggtgagaaattcaccgccgccgagttccgcgccaagtgccgcgaatacgcggcgacccaggttgacggtcaacgcaaagactttatccgtctgggcgtgctgggcgactggtcgcacccgtacctgaccatggacttcaaaactgaagccaacatcatccgcgcgctgggcaaaatcatcggcaacggtcacctgcacaaaggcgcgaagccagttcactggtgcgttgactgccgttctgcgctggcggaagcggaagttgagtattacgacaaaacttctccgtccatcgacgttgctttccaggcagtcgatcaggatgcactgaaagcaaaatttgccgtaagcaacgttaacggcccaatctcgctggtaatctggaccaccacgccgtggactctgcctgccaaccgcgcaatctctattgcaccagatttcgactatgcgctggtgcagatcgacggtcaggccgtgattctggcgaaagatctggttgaaagcgtaatgcagcgtatcggcgtgaccgattacaccattctcggcacggtaaaaggtgcggagcttgagctgctgcgctttacccatccgtttatgggcttcgacgttccggcaatcctcggcgatcacgttaccctggatgccggtaccggtgccgttcacaccgcgcctggccacggcccggacgactatgtgatcggtcagaaatacggcctggaaaccgctaacccggttggcccggacggcacttatctgccgggcacttatccgacgctggatggcgtgaacgtcttcaaagcgaacgacatcgtcgttgcgctgctgcaggaaaaaggcgcgctgctgcacgttgagaaaatgcagcacagctatccgtgctgctggcgtcacaaaacgccgatcatcttccgcgcgacgccgcagtggttcgtcagcatggatcagaaaggtctgcgtgcgcagtcactgaaagagatcaaaggcgtgcagtggatcccggactggggccaggcgcgtatcgagtcgatggttgctaaccgtcctgactggtgtatctcccgtcagcgcacctggggtgtaccgatgtcactgttcgtgcacaaagacacggaagagctgcatccgcgtacccttgaactgatggaagaagtggcaaaacgcgttgaagtcgatggcatccaggcgtggtgggatctcgatgcgaaagagatcctcggcgacgaagctgatcagtacgtgaaagtgccggacacattggatgtatggtttgactccggatctacccactcttctgttgttgacgtgcgtccggaatttgccggtcacgcagcggacatgtatctggaaggttctgaccaacaccgcggctggttcatgtcttccctaatgatctccaccgcgatgaagggtaaagcgccgtatcgtcaggtactgacccacggctttaccgtggatggtcagggccgcaagatgtctaaatccatcggcaataccgtttcgccgcaggatgtgatgaacaaactgggcgcggatattctgcgtctgtgggtggcatcaaccgactacaccggtgaaatggccgtttctgacgagatcctgaaacgtgctgccgatagctatcgtcgtatccgtaacaccgcgcgcttcctgctggcaaacctgaacggttttgatccagcaaaagatatggtgaaaccggaagagatggtggtactggatcgctgggccgtaggttgtgcgaaagcggcacaggaagacatcctcaaggcgtacgaagcatacgatttccacgaagtggtacagcgtctgatgcgcttctgctccgttgagatgggttccttctacctcgacatcatcaaagaccgtcagtacaccgccaaagcggacagtgtggcgcgtcgtagctgccagactgcgctatatcacatcgcagaagcgctggtgcgctggatggcaccaatcctctccttcaccgctgatgaagtgtggggctacctgccgggcgaacgtgaaaaatacgtcttcaccggtgagtggtacgaaggcctgtttggcctggcagacagtgaagcgatgaacgatgcgttctgggacgagctgttgaaagtgcgtggcgaagtgaacaaagtcattgagcaagcgcgtgccgacaagaaagtgggtggctcgctggaagcggcagtaaccttgtatgcagaaccggaactgtcggcgaaactgaccgcgctgggcgatgaattacgatttgtcctgttgacctccggcgctaccgttgcagactataacgacgcacctgctgatgctcagcagagcgaagtactcaaagggctgaaagtcgcgttgagtaaagccgaaggtgagaagtgcccacgctgctggcactacacccaggatgtcggcaaggtggcggaacacgcagaaatctgcggccgctgtgtcagcaacgtcgccggtgacggtgaaaaacgtaagtttgcctga

SEQ ID NO: 12 - DNA, 2583 bp, E. coli
  leuS Leucine-tRNA synthetase
atgcaagagcaataccgcccggaagagatagaatccaaagtacagcttcattgggatgagaagcgcacatttgaagtaaccgaagacgagagcaaagagaagtattactgcctgtctatgcttccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggtactatgcgcgctacacttgcccgcagtacaaagaaggtatgctggattccgaagcggctaactactggctgccggtggatatctacattggtggtattgaacacgccattatgcacctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 13 - DNA, 1518 bp, E. coli
  lysS Lysine-tRNA synthetase, constitutive
atgtctgaacaacacgcacagggcgctgacgcggtagtcgatcttaacaatgaactgaaaacgcgtcgtgagaagctggcgaacctgcgcgagcaggggattgccttcccgaacgatttccgtcgcgatcatacctctgaccaattgcacgcagaattcgacggcaaagagaacgaagaactggaagcgctgaacatcgaagtcgccgttgctggccgcatgatgacccgtcgtattatgggtaaagcgtctttcgttaccctgcaggacgttggcggtcgcattcagctgtacgttgcccgtgacgatctcccggaaggcgtttataacgagcagttcaaaaaatgggacctcggcgacatcctcggcgcgaaaggtaagctgttcaaaaccaaaaccggcgaactgtctatccactgcaccgagttgcgtctgctgaccaaagcactgcgtccgctgccggataaattccacggcttgcaggatcaggaagcgcgctatcgtcagcgttatctcgatctcatctccaacgatgaatcccgcaacacctttaaagtgcgctcgcagatcctctctggtattcgccagttcatggtgaaccgcggctttatggaagttgaaacgccgatgatgcaggtgatccctggcggtgccgctgcgcgtccgtttatcacccaccataacgcgctggatctcgacatgtacctgcgtatcgcgccggaactgtacctcaagcgtctggtggttggtggcttcgagcgtgtattcgaaatcaaccgtaacttccgtaacgaaggtatttccgtacgtcataacccagagttcaccatgatggaactctacatggcttacgcagattacaaagatctgatcgagctgaccgaatcgctgttccgtactctggcacaggatattctcggtaagacggaagtgacctacggcgacgtgacgctggacttcggtaaaccgttcgaaaaactgaccatgcgtgaagcgatcaagaaatatcgcccggaaaccgacatggcggatctggacaacttcgactctgcgaaagcaattgctgaatctatcggcatccacgttgagaagagctggggtctgggccgtatcgttaccgagatcttcgaagaagtggcagaagcacatctgattcagccgaccttcattactgaatatccggcagaagtttctccgctggcgcgtcgtaacgacgttaacccggaaatcacagaccgctttgagttcttcattggtggtcgtgaaatcggtaacggctttagcgagctgaatgacgcggaagatcaggcgcaacgcttcctggatcaggttgccgcgaaagacgcaggtgacgacgaagcgatgttctacgatgaagattacgtcaccgcactggaacatggcttaccgccgacagcaggtctgggaattggtatcgaccgtatggtaatgctgttcaccaacagccataccatccgcgacgttattctgttcccggcgatgcgtccggtaaaataa

SEQ ID NO: 14 - DNA, 1518 bp, E. coli
  lysU Lysine-tRNA synthetase, heat inducible
atgtctgaacaagaaacacggggagccaatgaggctattgattttaacgatgaactgagaaatcgccgcgaaaaactggcggcactacgtcagcaaggtgtggcgtttcccaatgattttcgccgcgaccatacctctgaccagttgcacgaagagtttgatgcgaaggataaccaggaactggaatccttaaacattgaagtctcggttgctggccgaatgatgacccgtcgtatcatggggaaagcctcctttgtaacgttgcaggatgtcggtggccgtattcaactgtacgttgcaagagatagcctgccagaaggtgtttataacgatcagtttaaaaaatgggatctgggtgacattatcggtgcccgcggtacgctgtttaagacgcaaacgggtgagctttccattcactgtactgagctgcgcctgctgactaaagcactacgtcctttaccagataaattccatggtctgcaggatcaggaagtccgttatcgtcaacgttatctggacctcatcgctaacgataaatcccgtcaaacgtttgttgtccgttcaaaaattctggccgctatccgtcaattcatggtcgcgcgcggctttatggaagtagaaaccccgatgatgcaggtaattccaggtggggcatctgctcgcccgtttattacccatcataatgctctggatttagatatgtatctgcgtatcgcgccggagctgtatctgaaacgtctggttgtaggcggttttgaacgggtattcgaaatcaaccgtaacttccgtaatgaaggtatttctgttcgccataatcctgagttcacaatgatggaactctacatggcgtatgcggattaccacgatttgattgaactgacagagtcactgttccgcaccctggcacaagaggttctgggtaccactaaagtcacttatggcgagcatgtgtttgatttcggcaaaccgtttgaaaaactcaccatgcgcgaagcaatcaaaaaatatcgtccagaaaccgatatggccgacctggataattttgatgctgctaaagcattagctgaatctatcggtattacggtagagaaaagctgggggttgggacgtattgtcacagagatctttgatgaagtggcagaagcacatctgattcagccaacctttattacggaatatccggcagaagtgtccccgctggcacgccgtaatgatgttaacccggaaatcaccgaccgttttgaattcttcatcggtggtcgtgaaatcggtaatggttttagcgaattaaacgacgcagaagatcaggctgaacgtttccaggaacaggttaatgctaaagctgcaggtgacgacgaagccatgttctatgacgaagattacgtgactgcgctggaatatggtctgccgccaaccgctggtctgggtattggtatcgaccgaatgattatgctgtttactaacagccatactattcgcgacgttattctcttcccggcgatgcgcccacagaaataa

SEQ ID NO: 15 - DNA, 2034 bp, E. coli
  metG Methionine-tRNA synthetase
atgactcaagtcgcgaagaaaattctggtgacgtgcgcactgccgtacgctaacggctcaatccacctcggccatatgctggagcacatccaggctgatgtctgggtccgttaccagcgaatgcgcggccacgaggtcaacttcatctgcgccgacgatgcccacggtacaccgatcatgctgaaagctcagcagcttggtatcaccccggagcagatgattggcgaaatgagtcaggagcatcagactgatttcgcaggctttaacatcagctatgacaactatcactcgacgcacagcgaagagaaccgccagttgtcagaacttatctactctcgcctgaaagaaaacggttttattaaaaaccgcaccatctctcagctgtacgatccggaaaaaggcatgttcctgccggaccgttttgtgaaaggcacctgcccgaaatgtaaatccccggatcaatacggcgataactgcgaagtctgcggcgcgacctacagcccgactgaactgatcgagccgaaatcggtggtttctggcgctacgccggtaatgcgtgattctgaacacttcttctttgatctgccctctttcagcgaaatgttgcaggcatggacccgcagcggtgcgttgcaggagcaggtggcaaataaaatgcaggagtggtttgaatctggcctgcaacagtgggatatctcccgcgacgccccttacttcggttttgaaattccgaacgcgccgggcaaatatttctacgtctggctggacgcaccgattggctacatgggttctttcaagaatctgtgcgacaagcgcggcgacagcgtaagcttcgatgaatactggaagaaagactccaccgccgagctgtaccacttcatcggtaaagatattgtttacttccacagcctgttctggcctgccatgctggaaggcagcaacttccgcaagccgtccaacctgtttgttcatggctatgtgacggtgaacggcgcaaagatgtccaagtctcgcggcacctttattaaagccagcacctggctgaatcattttgacgcagacagcctgcgttactactacactgcgaaactctcttcgcgcattgatgatatcgatctcaacctggaagatttcgttcagcgtgtgaatgccgatatcgttaacaaagtggttaacctggcctcccgtaatgcgggctttatcaacaagcgttttgacggcgtgctggcaagcgaactggctgacccgcagttgtacaaaaccttcactgatgccgctgaagtgattggtgaagcgtgggaaagccgtgaatttggtaaagccgtgcgcgaaatcatggcgctggctgatctggctaaccgctatgtcgatgaacaggctccgtgggtggtggcgaaacaggaaggccgcgatgccgacctgcaggcaatttgctcaatgggcatcaacctgttccgcgtgctgatgacttacctgaagccggtactgccgaaactgaccgagcgtgcagaagcattcctcaatacggaactgacctgggatggtatccagcaaccgctgctgggccacaaagtgaatccgttcaaggcgctgtataaccgcatcgatatgaggcaggttgaagcactggtggaagcctctaaagaagaagtaaaagccgctgccgcgccggtaactggcccgctggcagatgatccgattcaggaaaccatcacctttgacgacttcgctaaagttgacctgcgcgtggcgctgattgaaaacgcagagtttgttgaaggttctgacaaactgctgcgcctgacgctggatctcggcggtgaaaaacgcaatgtcttctccggtattcgttctgcttacccggatccgcaggcactgattggtcgtcacaccattatggtggctaacctggcaccacgtaaaatgcgcttcggtatctctgaaggcatggtgatggctgccggtcctggcgggaaagatattttcctgctaagcccggatgccggtgctaaaccgggtcatcaggtgaaataa

SEQ ID NO: 16 - DNA, 984 bp, E. coli
  pheS Phenylalanine-tRNA synthetase, alpha-subunit
atgtcacatctcgcagaactggttgccagtgcgaaggcggccattagccaggcgtcagatgttgccgcgttagataatgtgcgcgtcgaatatttgggtaaaaaagggcacttaacccttcagatgacgaccctgcgtgagctgccgccagaagagcgtccggcagctggtgcggttatcaacgaagcgaaagagcaggttcagcaggcgctgaatgcgcgtaaagcggaactggaaagcgctgcactgaatgcgcgtctggcggcggaaacgattgatgtctctctgccaggtcgtcgcattgaaaacggcggtctgcatccggttacccgtaccatcgaccgtatcgaaagtttcttcggtgagcttggctttaccgtggcaaccgggccggaaatcgaagacgattatcataacttcgatgctctgaacattcctggtcaccacccggcgcgcgctgaccacgacactttctggtttgacactacccgcctgctgcgtacccagacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatcagctttaccaacctgaaaggcacgctgcacgacttcctgcgtaacttctttgaggaagatttgcagattcgcttccgtccttcctacttcccgtttaccgaaccttctgcagaagtggacgtcatgggtaaaaacggtaaatggctggaagtgctgggctgcgggatggtgcatccgaacgtgttgcgtaacgttggcatcgacccggaagtttactctggtttcgccttcgggatggggatggagcgtctgactatgttgcgttacggcgtcaccgacctgcgttcattcttcgaaaacgatctgcgtttcctcaaacagtttaaataa

SEQ ID NO: 17 - DNA, 2388 bp, E. coli
  pheT Phenylalanine-tRNA synthetase, beta-subunit
atgaaattcagtgaactgtggttacgcgaatgggtgaacccggcgattgatagcgatgcgctggcaaatcaaatcactatggcgggcctggaagttgacggtgtagaaccggttgccggcagcttccacggcgtggtcgttggtgaagtggttgagtgtgcgcagcatccgaacgctgacaaactgcgtgtgacaaaagtgaatgtcggcggcgatcgcctgctggacatcgtctgcggtgcgccaaactgccgtcagggcctgcgtgtagcggtagcgaccattggtgctgttctgccgggtgatttcaaaattaaagcggcgaaactgcgtggcgaaccgtctgaagggatgctgtgctccttctctgaactgggcatttctgacgatcacagcggcattatcgaactgcctgcggatgcgccgattggcaccgatatccgtgaatacctgaaacttgatgacaacaccatcgaaatcagcgtgacgccaaaccgtgccgactgcttaggcatcattggtgttgcgcgtgacgttgccgtgctgaaccagctgccgctggttcaaccggaaatcgttccggttggtgcgaccatcgacgacacgctgccgattacagtcgaagcgccggaagcctgcccgcgttatcttggccgtgtggtaaaaggcattaacgttaaagcgccaactccgctgtggatgaaagaaaaactgcgtcgttgcgggatccgttctatcgatgcagttgttgacgtcaccaactatgtgctgctcgaactgggccagccgatgcacgctttcgataaagatcgcattgaaggcggcattgtggtgcggatggcgaaagagggcgaaacgctggtgctgctcgacggtactgaagcgaagctgaatgctgacactctggtcatcgccgaccacaacaaggcgctggcgatgggcggcatcttcggtggcgaacactctggcgtgaatgacgaaacacaaaacgtgctgctggaatgcgcgttctttagcccgctgtctatcaccggtcgtgctcgtcgtcatggcctgcataccgatgcgtctcaccgttatgagcgtggcgttgatccggcactgcagcacaaagcgatggaacgtgcgacccgtctgctgatcgacatctgcggtggtgaggctggcccggtaattgatatcaccaacgaagcaacgctgccgaagcgtgcaaccatcactctacgtcgtagcaaactggatcgcctgatcggccatcatattgcggatgagcaggtaactgacattctgcgtcgtctcggctgcgaagtgaccgaaggcaaagacgaatggcaggcagttgcgccgagctggcgtttcgatatggagattgaagaagatctggttgaagaagtcgcgcgtgtttacggctacaacaacatcccggatgagccggtacaggcaagcctgattatgggtactcaccgtgaagctgacctgtcgctcaagcgcgtgaaaacgctgctcaacgacaaaggctatcaggaagtgatcacctacagcttcgttgatccgaaagtgcagcagatgatccatccaggcgttgaagccttactgctgccaagcccgatctctgttgaaatgtcagcaatgcgtctttctctgtggactggcctgctggcaaccgtggtgtacaaccagaaccgtcagcagaaccgtgtgcgcattttcgaaagcggtctgcgtttcgtaccagatactcaggcaccgttgggcattcgtcaggatctgatgttagccggtgtgatttgcggtaaccgttacgaagagcactggaacctggcaaaagagaccgttgatttctatgatttgaaaggcgatcttgaatccgttctcgacctgaccggtaaactgaatgaggttgagttccgtgcagaagcgaatccggcactgcatccggggcaatccgcagcgatttatctgaaaggtgaacgtattggttttgttggggttgttcatcctgaactggaacgtaaactggatcttaacggtcgcactctggtgttcgaactggagtggaacaagctcgcagaccgcgtggtgcctcaggcgcgcgagatttctcgcttcccggcgaaccgtcgtgacatcgcggtggtggtcgcagaaaacgttcccgcagcggatattttatccgaatgtaagaaagttggcgtaaatcaggtagttggcgtaaacttatttgacgtgtaccgcggtaagggtgttgcggaggggtataagagcctcgccataagcctgatcctgcaagataccagccgtacactcgaagaagaggagattgccgctaccgtcgccaaatgtgtagaggcattaaaagagcgattccaggcatcattgagggattga

SEQ ID NO: 18 - DNA, 978 bp, E. coli
  lysyl-tRNA synthetase, genX
atgagcgaaacggcatcctggcagccgagcgcatccattcctaacttattaaaacgcgcggcgattatggcggagatccgtcgtttctttgccgatcgtggagtgctggaggtggagacgccttgtatgagccaggcgacggtaaccgatattcatttggtcccgtttgagacacgtttcgttggccccgggcattcgcaggggatgaatctctggttaatgaccagcccggaataccatatgaaacgcctgctggttgccggttgtgggccggtattccagctgtgccgcagcttccgtaatgaagagatggggcgttatcacaaccctgagttcactatgctggagtggtatcgaccgcactatgatatgtaccggttgatgaacgaggtggacgatctcttacaacaggtgctggactgcccggcagcagaaagcctttcttatcaacaagctttcttgcgttatctggaaattgacccgctctctgccgacaaaacgcaactgcgggaagtcgcagcgaaactggatttgagcaatgttgctgataccgaagaagaccgcgacacgctgctacaattgctgtttacctttggcgtagagccaaatattggcaaagaaaaaccgacctttgtgtaccactttccagccagccaggcatcactggcgcaaatcagtaccgaagatcatcgggtcgctgaacgctttgaggtttattataaaggtattgagctggcgaatggtttccatgaattgacggatgcccgtgagcagcaacaacgctttgaacaagataaccgtaagcgcgcggcgcgcggtttgccgcagcaccccattgaccagaatctgattgaagccttgaaagtcggtatgcctgactgttccggcgtggcattaggtgttgatcgtctggtgatgttggcgctgggcgcggagacactggctgaagtcatcgcctttagcgttgaccgggcataa

SEQ ID NO: 19 - DNA, 1719 bp, E. coli
  proS Proline-tRNA synthetase
atgcgtactagccaatacctgctctccactctcaaggagacacctgccgacgccgaggtgatcagccatcagctgatgctgcgcgccgggatgatccgcaagctggcctccgggttatatacctggctgccgaccggcgtgcgcgttctgaaaaaagtcgaaaacatcgtgcgtgaagagatgaacaacgccggtgcgatcgaggtgtcgatgccggtggttcagccagccgatttgtggcaagagagtggtcgttgggaacagtacggtccggaactgctgcgttttgttgaccgtggcgagcgtccgttcgtactcggcccaactcatgaagaagttatcactgacctgattcgtaacgagcttagctcttacaaacagctgccgctgaacttctatcagatccagaccaagttccgcgacgaagtgcgtccgcgtttcggcgtcatgcgttcccgcgaattcctgatgaaagatgcttactctttccatacttctcaggaatccctgcaggaaacctacgatgcaatgtatgcggcctacagcaaaatcttcagccgcatggggctggatttccgcgccgtacaagccgacaccggttctatcggcggcagcgcctctcacgaattccaggtgctggcgcagagcggtgaagacgatgtggtcttctccgacacctctgactatgcagcgaacattgaactggcagaagctatcgcgccgaaagaaccgcgcgctgctgctacccaggaaatgacgctggttgatacgccgaacgcgaaaaccatcgcggaactggttgaacagttcaatctgccgattgagaaaacggttaagactctgctggttaaagcggttgaaggcagcagcttcccgcaggttgcgctgctggtgcgcggtgatcacgagctgaacgaagttaaagcagaaaaactgccgcaggttgcaagcccgctgactttcgcgaccgaagaagaaattcgtgccgtggttaaagccggtccgggttcactgggtccggtaaacatgccgattccggtggtgattgaccgtaccgttgcggcgatgagtgatttcgctgctggtgctaacatcgatggtaaacactacttcggcatcaactgggatcgcgatgtcgctaccccggaagttgcagatatccgtaacgtggtggctggcgatccaagcccggatggccagggtaggctgctgatcaaacgtggtatcgaagttggtcacatcttccagctgggtaccaagtactccgaagcactgaaagcctccgtacagggtgaagatggccgtaaccaaatcctgacgatgggttgctacggtatcggggtaacgcgtgtggtagctgcggcgattgagcagaactacgacgaacgaggcatcgtatggcctgacgctatcgcgccgttccaggtggcgattctgccgatgaacatgcacaaatccttccgcgtacaagagcttgctgagaaactgtacagcgaactgcgtgcacaaggtatcgaagtgctgctggatgaccgcaaagagcgtccgggcgtgatgtttgctgatatggaactgatcggtattccgcacactattgtgctgggcgaccgtaacctcgacaacgacgatatcgaatataaatatcgtcgcaacggcgagaaacagttaattaagactggtgacatcgtcgaatatctggtgaaacagattaaaggctga

SEQ ID NO: 20 - DNA, 1293 bp, E. coli
  serS Serine-tRNA synthetase
atgctcgatcccaatctgctgcgtaatgagccagacgcagtcgctgaaaaactggcacgccggggctttaagctggatgtagataagctgggcgctcttgaagagcgtcgtaaagtattgcaggtcaaaacggaaaacctgcaagcggagcgtaactcccgatcgaaatccattggccaggcgaaagcgcgcggggaagatatcgagcctttacgtctggaagtgaacaaactgggcgaagagctggatgcagcaaaagccgagctggatgctttacaggctgaaattcgcgatatcgcgctgaccatccctaacctgcctgcagatgaagtgccggtaggtaaagacgaaaatgacaacgttgaagtcagccgctggggtaccccgcgtgagtttgactttgaagttcgtgaccacgtgacgctgggtgaaatgcactctggcctcgactttgcagctgcagttaagctgactggttcccgctttgtggtaatgaaagggcagattgctcgcatgcaccgcgcactgtcgcagtttatgctggatctgcataccgaacagcatggctacagtgagaactatgttccgtacctggttaaccaggacacgctgtacggtacgggtcaactgccgaaatttgctggcgatctgttccatactcgtccgctggaagaagaagcagacaccagtaactatgcgctgatcccaacggcagaagttccgctgactaacctggtgcgcggtgaaatcatcgatgaagatgatctgccaattaagatgaccgcccacaccccatgcttccgttctgaagccggttcatatggtcgtgacacccgtggtctgatccgtatgcaccagttcgacaaagttgaaatggtgcagatcgtgcgcccagaagactcaatggcggcgctggaagagatgactggtcatgcagaaaaagtcctgcagttgctgggcctgccgtaccgtaaaatcatcctttgcactggcgacatgggctttggcgcttgcaaaacttacgacctggaagtatggatcccggcacagaacacctaccgtgagatctcttcctgctccaacgtttgggatttccaggcacgtcgtatgcaggcacgttgccgcagcaagtcggacaagaaaacccgtctggttcataccctgaacggttctggtctggctgttggtcgtacgctggttgcagtaatggaaaactatcagcaggctgatggtcgtattgaagtaccagaagttctgcgtccgtatatgaacggactggaatatattggctaa

SEQ ID NO: 21 - DNA, 1929 bp, E. coli
  thrS Threonine-tRNA synthetase, autogenously regulated
atgcctgttataactcttcctgatggcagccaacgccattacgatcacgctgtaagccccatggatgttgcgctggacattggtccaggtctggcgaaagcctgtatcgcagggcgcgttaatggcgaactggttgatgcttgcgatctgattgaaaacgacgcacaactgtcgatcattaccgccaaagacgaagaaggtctggagatcattcgtcactcctgtgcgcacctgttagggcacgcgattaaacaactttggccgcataccaaaatggcaatcggcccggttattgacaacggtttttattacgacgttgatcttgaccgcacgttaacccaggaagatgtcgaagcactcgagaagcggatgcatgagcttgctgagaaaaactacgacgtcattaagaagaaagtcagctggcacgaagcgcgtgaaactttcgccaaccgtggggagagctacaaagtctccattcttgacgaaaacatcgcccatgatgacaagccaggtctgtacttccatgaagaatatgtcgatatgtgccgcggtccgcacgtaccgaacatgcgtttctgccatcatttcaaactaatgaaaacggcaggggcttactggcgtggcgacagcaacaacaaaatgttgcaacgtatttacggtacggcgtgggcagacaaaaaagcacttaacgcttacctgcagcgcctggaagaagccgcgaaacgcgaccaccgtaaaatcggtaaacagctcgacctgtaccatatgcaggaagaagcgccgggtatggtattctggcacaacgacggctggaccatcttccgtgaactggaagtgtttgttcgttctaaactgaaagagtaccagtatcaggaagttaaaggtccgttcatgatggaccgtgtcctgtgggaaaaaaccggtcactgggacaactacaaagatgcaatgttcaccacatcttctgagaaccgtgaatactgcattaagccgatgaactgcccgggtcacgtacaaattttcaaccaggggctgaagtcttatcgcgatctgccgctgcgtatggccgagtttggtagctgccaccgtaacgagccgtcaggttcgctgcatggcctgatgcgcgtgcgtggatttacccaggatgacgcgcatatcttctgtactgaagaacaaattcgcgatgaagttaacggatgtatccgtttagtctatgatatgtacagcacttttggcttcgagaagatcgtcgtcaaactctccactcgtcctgaaaaacgtattggcagcgacgaaatgtgggatcgtgctgaggcggacctggcggttgcgctggaagaaaacaacatcccgtttgaatatcaactgggtgaaggcgctttctacggtccgaaaattgaatttaccctgtatgactgcctcgatcgtgcatggcagtgcggtacagtacagctggacttctctttgccgtctcgtctgagcgcttcttatgtaggcgaagacaatgaacgtaaagtaccggtaatgattcaccgcgcaattctggggtcgatggaacgtttcatcggtatcctgaccgaagagttcgctggtttcttcccgacctggcttgcgccggttcaggttgttatcatgaatattaccgattcacagtctgaatacgttaacgaattgacgcaaaaactatcaaatgcgggcattcgtgttaaagcagacttgagaaatgagaagattggctttaaaatccgcgagcacactttgcgtcgcgtcccatatatgctggtctgtggtgataaagaggtggaatcaggcaaagttgccgttcgcacccgccgtggtaaagacctgggaagcatggacgtaaatgaagtgatcgagaagctgcaacaagagattcgcagccgcagtcttaaacaattggaggaataa

SEQ ID NO: 22 - DNA, 1005 bp, E. coli
  trpS Tryptophan-tRNA synthetase
atgactaagcccatcgtttttagtggcgcacagccctcaggtgaattgaccattggtaactacatgggtgcgctgcgtcagtgggtaaacatgcaggatgactaccattgcatttactgtatcgttgaccaacacgcgatcaccgtgcgccaggatgcacagaagctgcgtaaagcgacgctggatacgctggccttgtatctggcttgtggtatcgatcctgagaaaagcaccatttttgttcagtcccacgtgccggaacatgcacagttaggctgggcactgaactgctatacctacttcggcgaactgagtcgcatgacgcagtttaaagataaatctgcgcgttatgccgagaacatcaacgctggtctgtttgactatccggtgctgatggcagcggacatcctgctgtatcaaactaatctggtaccggtgggtgaagaccagaaacagcacctcgaactgagccgcgatattgcccagcgtttcaacgcgctgtatggcgagatctttaaggtgccggagccgtttattccgaaatctggcgcgcgcgtaatgtcgctgctggagccgaccaagaagatgtccaagtctgacgataatcgcaataacgttatcggcctgctggaagatccgaaatcggtagtgaagaaaatcaaacgtgcggtcactgactccgacgagccgccggtagttcgctacgatgtgcagaacaaagcgggcgtttccaacctgttggatatcctttcagcggtaacgggccagagcatcccagaactggaaaaacagttcgaaggcaagatgtatggtcatctgaaaggtgaagtggctgatgccgtttccggtatgctgactgaattgcaggaacgctatcaccgtttccgcaacgatgaagccttcctgcaacaggtgatgaaagatggcgcggaaaaagccagcgcgcacgcttcccgtacgctaaaagcggtgtacgaagcgattggttttgtggcgaagccgtaa

SEQ ID NO: 23 - DNA, 1275 bp, E. coli
  tyrS Tyrosine-tRNA synthetase
atggcaagcagtaacttgattaaacaattgcaagagcgggggctggtagcccaggtgacggacgaggaagcgttagcagagcgactggcgcaaggcccgatcgcgctctattgcggcttcgatcctaccgctgacagcttgcatttggggcatcttgttccattgttatgcctgaaacgcttccagcaggcgggccacaagccggttgcgctggtaggcggcgcgacgggtctgattggcgacccgagcttcaaagctgccgagcgtaagctgaacaccgaagaaactgttcaggagtgggtggacaaaatccgtaagcaggttgccccgttcctcgatttcgactgtggagaaaactctgctatcgcggcgaacaactatgactggttcggcaatatgaatgtgctgaccttcctgcgcgatattggcaaacacttctccgttaaccagatgatcaacaaagaagcggttaagcagcgtctcaaccgtgaagatcaggggatttcgttcactgagttttcctacaacctgttgcagggttatgacttcgcctgtctgaacaaacagtacggtgtggtgctgcaaattggtggttctgaccagtggggtaacatcacttctggtatcgacctgacccgtcgtctgcatcagaatcaggtgtttggcctgaccgttccgctgatcactaaagcagatggcaccaaatttggtaaaactgaaggcggcgcagtctggttggatccgaagaaaaccagcccgtacaaattctaccagttctggatcaacactgcggatgccgacgtttaccgcttcctgaagttcttcacctttatgagcattgaagagatcaacgccctggaagaagaagataaaaacagcggtaaagcaccgcgcgcccagtatgtactggcggagcaggtgactcgtctggttcacggtgaagaaggtttacaggcggcaaaacgtattaccgaatgcctgttcagcggttctttgagtgcgctgagtgaagcggacttcgaacagctggcgcaggacggcgtaccgatggttgagatggaaaagggcgcagacctgatgcaggcactggtcgattctgaactgcaaccttcccgtggtcaggcacgtaaaactatcgcctccaatgccatcaccattaacggtgaaaaacagtccgatcctgaatacttctttaaagaagaagatcgtctgtttggtcgttttaccttactgcgtcgcggtaaaaagaattactgtctgatttgctggaaataa

SEQ ID NO: 24 - DNA, 2856 bp, E. coli
  valS Valine-tRNA synthetase
atggaaaagacatataacccacaagatatcgaacagccgctttacgagcactgggaaaagcagggctactttaagcctaatggcgatgaaagccaggaaagtttctgcatcatgatcccgccgccgaacgtcaccggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctgtaa

SEQ ID NO: 25 - DNA, 2634 bp, B. subtilis
  alanyl-tRNA synthetase
atgaaacacttaacttctgcggaagtgcgtcaaatgtttttggatttctttaaagaaaaaggacatgcggtagagccaagcgcgtcattagtgcctcatgaggatccttcactgctttggatcaacagcggtgttgcgacgctgaaaaaatattttgacggccgtgtcgtgccggaaaatccaagaatcgtaaacgctcaaaaagcgatcagaacaaacgatatagaaaatgtaggtaaaactgcgcgccatcacactttctttgaaatgctcggaaacttttccatcggcgattatttcaaagaagaagccattacatgggcttgggagtttttaacgagcgacaagtggattggcttcgacaaagagcttctctctgttacggttcatcctgaagatgaagaggcatatgagttttgggcgaaaaaaatcggtattcctgaagaaagaattatccgtctggaagggaacttctgggatatcggtgaagggccgagcggaccgaatacggaaatcttttacgaccgcggtgaagcatacggtaatgatccagaagatccggagctttacccaggcggggaaaacgaccgttacctggaagtatggaaccttgtgttctcagagttcaaccataaccctgacggcacgtacacaccgcttccaaagaaaaatattgatacaggcatgggtcttgaaagaatggtgtctgtcatccagaatgttccgacaaactttgacaccgatttgtttgttccgatcattaaagcaacagaatcgatttctggtgaaacatatggcaaagacaatgtgaaagacactgcgtttaaagtgattgctgaccatatcagaacggttgcctttgctgtcagtgacggtgcgctgccgtcaaatgaaggccgcggctatgtattaagacgcttattacgccgtgccgtgcgttatgccaaaacgatcaacatccatcgtccgttcatgtttgatttagtgccggttgtcgcagaaatcatggctgatttctatcctgaagtgaaagagaaagcggatttcatcgcaaaagtcattaaaaccgaggaagaacgcttccatgagacccttaatgaagggcttgcgatcctgtcagaaatgatcaaaaaagaaaaagacaagggcagcagcgttatctcaggtgctgatgtgtttaaactgtatgatacgtatggattcccggttgaattgactgaagaatacgcagaagacgagaacatgacggttgaccatgagggctttgaggaggaaatgaaccagcagcgtgaacgggcaagaaacgcccgccaggatgtcggcagcatgcaggtgcagggcggcgcactgcgcgacgtgacagttgaaagcacatttgtcggctactctcaaaccaaagctgatgccaatatcattgtgcttcttcaggacggccaattaatcgaagaggctcatgaaggagaaagcgttcaaatcattcttgatgaaacaccgttctatgcagaaagcggcggccaaatcggcgacaaaggctatctccgaagcgagcaggcagttgtaagaattaaagatgttcaaaaagcgccgaacggccagcatgtgcatgaaggtgttgtggagagcggtactgttcaaaaaggcctgcatgttacggctgaagttgaagaccatatgagaagcggcgtcattaaaaaccacacggcaacgcatttattgcatcaggctctaaaagatgttcttggaactcatgtcaatcaggcgggctctcttgtaacggaaaaccgccttcgttttgacttctcgcactttggccaagtgacaaaagaagagctcgaacaaattgaaagaatcgtaaacgaaaagatctgggcgagcatcccggtcagcattgatttgaaaccgatcgctgaagcaaaagaaatgggtgcgatggcactgttcggcgaaaaatatggcgatattgtccgtgtcgttcaagttggagattacagcttagagctgtgcggcggctgccacgtcagaaatacagcagaaatcggcttgtttaaaatcgtttctgaatccggaatcggagcaggcacaagacggattgaagctgtaacgggacaaggcgcttacgtcgaaatgaacagccagatttctgtattgaagcagaccgctgacgagctgaaaacaaatatcaaagaagtgccgaaacgggtcgcggctctgcaggctgaactgaaggatgcacaaagagaaaatgaatctcttcttgcaaaactaggcaacgtggaagcaggagcaatcctgtcgaaagtaaaagaagttgacggcgtaaacgtgcttgcggcaaaagtaaatgcaaaagacatgaatcatctccgcactatggtcgatgaactcaaagcaaagcttggctctgcggtgatcgtgcttggtgcggtacaaaacgataaagttaatatctctgccggagtcacaaaggacctcattgagaaaggacttcacgccggcaagctggttaaacaagctgcggaagtttgcggcggaggcggcggaggccgtccggacatggcgcaggcaggcggtaaacagccggaaaaattagaagaagctttggcttctgtagaagattgggtgaaatccgtttta

SEQ ID NO: 26 - DNA, 1668 bp, B. subtilis
  arginyl-tRNA synthetase
atgaacattgcggaacaaatgaaggacgtgctgaaagaagaaatcaaagcggccgttctgaaagcgggactggctgaagaaagccagattcccaatgttgttttagaaacaccgaaagataaaacacacggcgactactcaacgaatatggcgatgcagttggcaagagtggcaaaaaaagcgccgcgtcaaatcgctgaagagattgtcgcccattttgataaagggaaggcttcgattgaaaaactggatatcgccggcccgggttttatcaatttctatatgaacaatcaatatttaacaaagctgattccgtctgtactggaagcaggggaagcatacggggaaacgaacatcggaaacggtgaacgagttcaggtagaattcgtatcagcgaatccgacgggagaccttcacctcgggcatgcgcgcggggcggctgtcggcgattccttgtgcaacgtgctttcaaaagcgggttacgatgtaagccgcgaatactacattaacgatgcgggaaaccaaatcaacaatctggcgctttctgttgaagtccgttactttgaagcactcggattggaaaaaccgatgccggaggatggctaccgcggtgaggatatcattgcgatcggaaagcgtctcgctgaggaatacggtgatcgtttcgttaacgaagaggaaagtgaacgcctcgcgtttttccgtgaatacggcctgaaatacgagctggaaaagcttcgcaaggacttggaaaacttccgtgtgccgtttgatgtgtggtattcagaaacttcactgtaccaaaatggaaaaatcgacaaggcgcttgaagcgctccgcgaaaaaggccacgtctatgaagaagacggcgcgacttggttccgttccacgactttcggcgatgataaagaccgcgtattgatcaagaaggacggcacgtacacgtaccttcttcctgatatcgcgtatcataaggacaagcttgaccgcggctttgataagctgatcaacgtttggggcgccgatcaccacggctacatcccgcgtatgaaagcggcaatcgaagcgctgggctatgaaaaaggaacgcttgaagtagaaatcattcagctcgttcacctttacaaaaacggcgagaaaatgaaaatgagcaaacggaccggtaaagccgtaacgatgcgtgacctgattgaagaagtcggcttggatgccgtgcgttacttctttgcaatgcgaagtgcagacacccatatggattttgacttagatcttgctgtatcaacatctaacgaaaaccctgtgtattatgcacaatacgcgcatgcccgtatttgcagcatgcttcgccaaggggaagagcaggggcttaaaccggcggcagatcttgatttcagccatattcaatcagaaaaagaatacgatctgctgaaaacgatcggcggcttcccagaggcagtggcggaagcagcggaaaaacgaattccgcaccgtgtcactaactatatttatgatctggcttctgctctgcacagcttctacaacgcagagaaagtcatcgaccctgaaaatgaagagaaaagccgcgcgcgcctggctttaatgaaggcaacgcaaattacgctgaacaatgcgcttcagctgatcggcgtatcggctccggaaaaaatg

SEQ ID NO: 27 - DNA, 1290 bp, B. subtilis
  asparaginyl tRNA synthetase
ttgaaaacaacaatcaaccaagtgtacaagcacgtaggtgaggaagtaacgatcggagcttgggtcgctaataagcgttcaagcgggaaaattgcgtttttacagcttcgggacggtaccggttttattcagggtgtcgtagtaaaagcggaagtggaagaaagcattttccaaacagctaaatcagtgacgcaggaaacgtcgctctatataaaagggattgtcaaagaggacgagcgttctccgcttggatatgaacttgctgtgacagatattgaagtcattcacgaagcgaccgattatccaattacaccaaaagaacacggaacggaatttttgatggatcacagacatttatggctgcgttcaaagcgccagcatgcgatcatgaaaatccgtaatgaaatcattcgcgcgacttacgaattctttaataacgaaggcttcgtaaaagtggatccgccgattttgactggaagcgcacctgaaggaacaacagaactctttgcgacaaagtactttgatgaagatgcatatctgtctcaaagcggacagctctacatggaagctgcggcaatggctttaggaaaagtattctctttcggaccgacattcagagcggaaaagtctaaaacaaagcgtcacttaatcgaattctggatgatcgaaccggaaatggcgtttgtagaatttgaagaaaaccttcaagtacaggaaaattacgtttctttcatcgtgcaatcagttcttaaaaattgcaaaattgaactaaacacattgggaagagacacgtcaaaacttgagcaaatcaaagccccgttcccaagaattacgtatgatgaagcgattgaatttctgaaagaaaaaggctttgacgatatcgagtggggagatgatttcggagcgcctcatgaaacagcgattgctgaacattatgacaaaccggtattcatcactcgctatccgacgtccttaaaaccgttctatatgcagccggcttctgaccgtgaggacgttgtgctttgcgctgacttgattgcgccggaaggctatggagaaatcatcggcgggtctgaacggattcacgatatggaacttttggaatcgcgtcttaaagaacatggactggattctgacgcttataaatggtatgctgaacttagaaaatatggatcagttcctcattccggcttcggccttggattagagcggacagtagcttggatcagcggagcgcctcacgttcgtgaaacgattccgttcccaagactgttaaaccgtctgtatccg

SEQ ID NO: 28 - DNA, 1776 bp, B. subtilis
  aspartyl-tRNA synthetase
ttgtttggaagaacatattattgcggtgatataactgaaaaagcaattggcgaatctgtaacgctgaaaggctgggtccaaaaaagacgagacctcggcggattgatttttattgacttgcgtgaccgtacgggcattgttcaagtcgtttttaaccctgatgtgtcaaaagaagcgcttgctattgcggaaggcatcagaaatgaatacgtgcttgatattcaaggaaaagtggtggcgcgtgaagagggaacagttaatccgaatttgaaaacaggcgcgatcgaaatacatgctgatggagtgaacgtattaaatgctgcaaagacacctccatttgcgatttctgatcaagctgaagaagtgtcagaggacgttcgtttaaaacaccgttacttagatttgcgcagaccggctatgttccagacgatgcagctgcgccataacgtgacgaaagctgttcgcagctttttagatgaaaatgggtttcttgatattgaaacgccgattttaacaggaagcacgcctgaaggcgcacgtgactacttggtgccgagccgtgtgcacgaaggtgagttctacgcgctgccgcagtctccgcagctatttaaacagcttctgatggtatcaggcattgaacgatattatcaaattgcccgctgtttccgcgatgaagacttgcgtgcggaccgtcagcctgagtttacgcaaattgatattgaaatgtcctttatgagccaggaagacattatgtcattggctgaggagatgatggcaaaggttatgcgtgaaacaaaaggtgaggaacttcagcttcctcttcctcgaatgacgtatgacgaagcaatgaacaagtacgggtctgataaaccggatacgcgttttgacatgcttttgacggatgtgtctgacattgtaaaggatacggaatttaaagtgttctcatcagctgtagcaaacggcggcgtggtcaaagccatcaatgtaaaaggcggtgccggcgattactccagaaaagacatcgacgctcttggcgcttttgctgcaaactacggggcaaaagggcttgcctgggtgaaggttgaggcagacggagtgaaaggcccgatcgctaaattctttgatgaagaaaagcagtctaagctgattgaagcacttgatgctgctgaaggtgatttgctgctgttcggagcggatcaattcgaagttgtggccgcatcactcggcgccctgcgcttaaagcttgggaaagaacgcggactgattgacgaaaaattgttcaatttcttatgggtcatcgactggccgctattagagcatgatccggaagaaggccgtttttacgcagcgcaccatccgttcacaatgcctgtccgagaagaccttgagctgattgaaacagcgcctgaagacatgaaagcgcaagcttatgacctcgtcttaaacggctatgagcttggcggcggttcaatccgtattttcgaaaaggatattcaggaaaaaatgtttgcgcttctcggcttctcaccagaagaagcggctgaacagttcggattcctgctggaagcatttgaatacggcgctcctccgcacggcggaatcgcactcggcttagaccgtctcgttatgcttctcgctggacgtacgaacctgagagatacgattgcattcccgaaaacggcaagtgcaagctgcctgatgacagaagcgccaggcgaagtaagcgacgcccagcttgacgagcttcatctttctatcaagaaaaaagttaagaac

SEQ ID NO: 29 - DNA, 1398 bp, B. subtilis
  cysteinyl-tRNAsynthetase
atgacaatcacactttataatacattgactagacagaaggaaacattcgttcctcttgaagagggaaaagtgaaaatgtatgtatgcggacccacggtttacaattacattcatatcgggaacgcgcgtccggcaatcgtttacgatacggttcgaaactatttagagtataaaggctatgatgtgcagtatgtctctaacttcacagacgtagacgataaattaattaaagcggcaaatgaactcggtgaggatgtacccaccatttcagagcgttttattaaagcatactttgaagacgtaggtgcgctcggctgccgaaaagccgaccttcatccgcgagtaatggagaacatggatgccattatcgaattcgtagatcagctcgtgaaaaagggctacgcatatgaatcagaaggtgacgtatatttcaaaaccagagcatttgaagggtacggaaagctttctcagcaatcaatcgatgaactaagatcaggtgcacgcatccgggtcggcgagaaaaaagaagatgctcttgatttcgcactgtggaaagcggcaaaagaaggagaaatctcttgggatagcccttgggggaaagggcgtccgggctggcacattgaatgctcagcaatggtgaaaaagtatctcggtgaccagattgatatccatgcgggcggacaggatttaacattccctcaccatgaaaacgaaattgcgcaatctgaggcgctgacaggcaaaacgtttgcgaagtactggcttcataatggttatatcaatattgataatgaaaaaatgtcaaaatcactaggcaactttgtgcttgtgcatgacatcattaaacagcatgatccgcagcttttgagattctttatgctatctgttcattatcgccatccgattaactattcagaagagcttctggagaatacgaaaagcgcgttcagccgtttaaaaacagcgtacagcaatcttcagcaccgtctgaacagcagtacgaatttaaccgaagatgacgatcaatggcttgaaaaggttgaagaacaccgcaaagcattcgaagaagagatggacgatgattttaatacggcgaatgccatttcagtcttgtttgacttagcgaaacacgccaattattatcttcagaaagatcatacggctgatcatgtgattacggcgtttattgagatgtttgaccgcattgtttctgtcctcggtttttcgttgggtgagcaggaacttctcgatcaagagattgaagacttaatcgaaaagcgaaatgaagcgcgccggaatcgcgattttgcattgtcagaccagatccgcgaccagctgaaaagcatgaatatcattcttgaagatacggctcaaggcactcgctggaaacggggagaa

SEQ ID NO: 30 - DNA, 1449 bp, B. subtilis
  glutamyl-tRNA synthetase
atgggaaacgaagtacgcgtccgttatgcaccgagtccaaccggacatttgcatattggaaatgccagaacggcgctttttaattatttatttgcccgcaatcaaggcggtaagtttatcattcgagttgaggatactgataaaaagcgcaatattgagggcggagaacaaagccagctgaattatctgaagtggctaggtattgactgggatgagagtgtggatgtcggaggagagtacggtccataccgtcagtcagagcgtaacgatatctataaagtgtactatgaagagcttcttgaaaaagggcttgcttataaatgttactgtacggaagaagagcttgaaaaagagcgtgaagaacagattgcccgcggagaaatgcctcgttattccggaaaacacagagacctgactcaggaagaacaggagaaatttatcgccgaaggcagaaaaccaagtattcgtttccgtgtgccggaaggaaaagtcatcgccttcaacgacatcgtaaaaggcgaaatttcttttgaatcagatggcatcggcgacttcgttattgtgaaaaaggacggaacgcctacttataacttcgcggtagctattgatgactacttaatgaaaatgacacacgtgctgcgcggtgaggatcatatttctaacacaccgaaacagattatgatctatcaagcattcgggtgggatattcctcagttcggacacatgacgctgattgtaaacgaaagccgtaaaaagctcagcaaacgtgatgaatccattattcaattcatcgagcagtacaaagagcttggctacctgccagaagcgctgttcaactttatcggcttgttaggctggtcaccggttggagaagaagagcttttcacaaaagagcagtttattgaaatttttgatgtaaatcgtttatctaaatcaccagctttgtttgatatgcataagctaaaatgggttaacaaccaatatgtgaagaagctggatcttgatcaggttgttgaactgacgcttccgcatttgcaaaaagccggcaaagttggcactgagctttctgctgaagaacaagaatgggttcgtaaactgatttccctgtatcatgagcaattaagctacggtgcggaaattgttgagctgactgatttgttctttacggatgagatcgagtataatcaagaagcgaaagctgttctggaagaagaacaggttcctgaagtgctcagcacattcgcagcgaagcttgaagagcttgaggagttcactccggataatatcaaagcatcgatcaaagcagtgcagaaagaaactggccataaagggaaaaaactgtttatgccgattcgtgttgctgtaacagggcaaactcacggtccggaactgccgcaatcaattgaattgatcggtaaagagactgcaattcagcgtttaaagaatatc

SEQ ID NO: 31 - DNA, 1458 bp, B. subtilis
  glutamyl-tRNA amidotransferase, subumit a
atgtcattatttgatcataaaatcacagaattaaaacagctcatacataaaaaagagattaagatttctgatctggttgatgaatcttataaacgcatccaagcggttgatgataaggtacaagcctttttggcattagatgaagaaagagcgcgcgcatacgcgaaggagcttgatgaggcggttgacggccgttctgagcacggtcttcttttcggtatgccgatcggcgtaaaagataatatcgtaacaaaagggctgcgcacaacatgctccagcaaaattctcgaaaactttgatccgatttacgatgctactgtcgttcagcgccttcaagacgctgaagcggtcacaatcggaaaactgaacatggacgaattcgccatggggtcatctacagaaaactcagcttacaagctgacgaaaaacccttggaacctggatacagttcccggcggttcaagcggcggatctgcagctgcggttgctgcgggagaagttccgttttctcttggatctgacacaggcggctccatccgtcagccggcatctttctgcggcgttgtcggattaaaacctacatacggacgtgtatctcgttacggcctggtcgcatttgcgtcttcattggaccaaatcggaccgattacacgtacggttgaggataacgcgtttttacttcaagcgatttccggcgtagacaaaatggactctacgagtgcaaatgtggacgtgcctgattttctttcttcattaactggcgacatcaaaggactgaaaatcgccgttccgaaagaataccttggtgaaggtgtcggcaaagaagcgagagaatctgtcttggcagcgctgaaagtccttgaaggtctcggcgctacatgggaagaagtgtctcttccgcacagtaaatacgcgcttgcgacatattacctgctgtcatcttctgaagcgtcagcgaaccttgcacgctttgacggcatccgctacggctaccgcacagacaacgcggataacctgatcgacctttacaagcaaacgcgcgctgaaggtttcggaaatgaagtcaaacgccgcatcatgctcggaacgtttgctttaagctcaggctactacgatgcgtactacaaaaaagcgcaaaaagtgcgtacgttgattaagaaggatttcgaggacgtatttgaaaaatatgatgttattgttggaccgactacaccgacacctgcgtttaaaatcggtgaaaacacgaaggatccgctcacaatgtacgcaaacgatatcttaacgattccggtcaaccttgccggcgtaccgggaatcagtgtgccatgcggattagcagacggacttccgctcggcctgcaaatcatcggaaaacactttgatgaaagcactgtataccgcgttgctcatgcatttgaacaagcaacagaccatcataaagcaaaacctgaactgtaa

SEQ ID NO: 32 - DNA, 885 bp, B. subtilis
  glycine tRNA synthetase, alpha subunit
atgaatattcaagacatgattctaaccttgcaaaagcattggtccagtcagggctgtgtgcttatgcaggcttacgatgtagaaaaaggagccggcacgatgagcccgtatacatttttgcgcagtatcggtcctgagccgtggaaagtggcttatgtagagccttccagacgtccggcagacggccgctacggggagaacccgaacagactgtatcagcatcatcagttccaggtcattattaaaccgtcacctgataacattcaagagctgtatttggattccttgcgtgctcttggaattgatccgcttgagcacgatattcgctttgttgaagacaactgggagaatccgtctttaggctgcgcgggtctaggctgggaagtttggcttgacggaatggaaataacacaatttacgtatttccagcaggtcgggggattagagtgtaaacccgtttctgtagagattacgtatggaattgagcgtctcgcgtcttatatccaggataaagaaaacgtgtttgacttggaatggacgtcagggtttacagtaaaagatttattcatgatggctgaatatgagcattctgtttatacgtttgaaacatcagacgtcgatatgctgttccaattgttcagcacatatgaaaaagaagcgatcaagcaaatggacaacggacttgttcatccagcatatgactatgtgctgaaatgctcgcacactttcaacctgcttgatgccaaaggtgcgatctctgttaccgagcggacgggctatatcgcaagagtgcggaatttagctagaaaagtagcaaaaacctactatgaggaacgagaaaaactagggttcccaatgcttaaaggggagggttcttctcatgag

SEQ ID NO: 33 - DNA, 2037 bp, B. subtilis
  glycine tRNA synthetase, beta subunit
atgagtaaacaggatttacttttagagatcggattagaggaaatgccggcgcgctttttgaatgaaagcatggttcagcttggcgacaagctgacaggctggcttaaagaaaaaaatattactcacggtgaagtgaaactcttcaatacaccgagacgtcttgctgtgttcgttaaggatgtcgcagaaaagcaggatgatataaaagaagaagcaaaagggcctgcgaaaaaaattgcccttgatgcggacgggaactggacaaaagcggcaatcggcttttcaaaaggccaaggcgcaaatgtggaagacctgtacatcaaagaagtaaaaggcatagagtatgtattcgtgcaaaaattccaagccggccaagaaacgaagtcgcttctgccagaattgagcggtttaattacaagcttgcatttcccgaaaaacatgcgctggggaaatgaagatttgcgttatatccgaccgatcaaatggattgtcgcattatttggacaggatgtcattccattttcgatcacaaacgttgagtctggacggacaacacagggacaccgtttcctcggacatgaagtgtcaattgaatcaccttcagcttatgaagagcagctcaaaggacagcatgttattgctgatcctagcgtccgaaagcaaatgattcaatctcagctggaaacaatggctgctgaaaacaattggagcattccagttgacgaagaccttcttgatgaagtgaatcatttagtagaatacccgacagctctttacggttcatttgaatccgagtttctgtcaattcctgaagaagtgcttgtcacgacaatgaaagagcatcagcgctacttccctgtcaaagacaaaaacggtgacctgctgcctcactttatcacagtccgaaacggcaacagccacgcgattgaaaatgtggcccgaggcaatgaaaaagtgctgcgcgcacgtttatctgatgcttcattcttctacaaagaagatcagaaactaaacattgatgcaaatgtgaagaagcttgaaaatatcgttttccatgaagagcttggttctcttgctgataaggttaggagagtcacatcaatcgcagagaaacttgccgttcgtcttcaggctgatgaagatacattaaaacatgtgaagcgggctgctgaaatttctaaattcgaccttgtgacgcatatgatatatgaattccctgaacttcaaggaattatgggcgaaaagtatgcgagaatgcttggcgaagatgaagctgtggctgcggcagttaatgaacattatatgcctagatcagcgggcggagaaacgccttctactttcaccggagctgttgtagcaatggcggataagcttgacacaatcgcttcattcttctctatcggcgtaattccgaccggttcccaggacccttacggactgccccgccaagcaagcggtattgttgcgattctccttgaccgcaactggggaatttcatttgaagagctgcttactttcgttcaaaccgataaagaaaatgaactgctggatttcttcactcagcgcttgaaatatgtcttgaatgctgaacaaatcagacatgatgtcattgacgccgtgctggaaagctctgagcttgagccgtactctgcgctgcataaagcacaagtactggagcaaaagcttggtgcacctggctttaaggaaacagccgaagcgttaggccgagtgatctctatcagcaaaaaaggagtccgcggagacattcagcctgatctgtttgaaaatgaatacgaagcaaaactgtttgatgcctaccaaacagcgaaggaaaatctgcaggaaaacttcagcaaaaaagattatgaggcggcgcttgcttcacttgcagccttaaaagaaccgatcgatgcttacttcgatcatacaatggttatagcggataatgagtcattaaaggcaaatcgtttagcgcaaatggtaagcttggcggatgagatcaagtcctttgcgaatatgaatgcccttattgtaaaa

SEQ ID NO: 34 - DNA, 1272 bp, B. subtilis
  histidyl tRNA synthetase
atgggatataacattccgagaggaacacaggatattctgcctggagaatcagatcgctggcagtttgttgaacaaattatgagagacacttgccgcacttatcaatataaagaaatccgcacaccgatttttgagcatacagaactgtttgccagaggcgtcggagaatcaacggatatcgtacaaaaagaaatgtataccttcgaggaccgcaaaggcagaagcctgacgcttcgtccggagggaacagctgctgcggttcgcgcttttaatgaaaacaagctgttcgcaaatcctgtacagccgacaaagctttattatgtcgggccgatgttccgttatgaacgtcctcagacgggccgttaccgccagttttatcagtttggaattgaagcgattggttcgaaagaccctgcaatcgacgctgaagtaatggcgcttgcgatgagtatttatgaaaaagcaggtttagaaaacgttaagcttgtcataaacagtctcggcgatcaagatagccgcaaaagctacagagaagcgctcgtgaaacacttcgagcctcgcatcgaagaattttgttcagactgccagtcccggctgcacacgaacccgcttagaattttggactgcaaaaaagaccgtgatcatgagctgatgaaatcggcaccttcgattttgacatatttaaatgaagaatcagccgcttattttgaaaaagtgaaacaatacttaaatgatcttggcatttcgtatgaaatcgatccgaaccttgtgagagggctggattattacaaccacaccgcatttgaaattatgagcaatgcggagggctttggcgcgattacgacacttgccggcggcggacgctacgacgggcttgtcgaacagatcggcggacctgaagcaccgggcatcggatttgcgatgagcatcgaacgcttgcttgccgcaattgatgctgaaaaaagagagctgcctgttgataagggaatcgactgctatatcgtcacactcggcgaaaaagcaaaggattattctgtttcattggtgtacaaactgagagaagcaggcatttccagtgaaatcgattatgaaaataagaaaatgaaaggccagtttaaaacagctgatcgcttaaaggccagatttattgcgattttgggtgaagatgagcttgcccaaaacaaaatcaatgtaaaggatgcacagacgggcgaacagattgaagtggcacttgatgaatttatacatgtgatgaaggcaaaccaaaaggga

SEQ ID NO: 35 - DNA, 2763 bp, B. subtilis
  isoleucyl-tRNA synthetase
atggattttaaagacacgctcttaatgccgaaaacagatttcccgatgcgtggaaatttgccaaaccgtgagcctgacattcaaaaaaaatgggaggaagaagatatctaccgtcttgttcaggaacggacgaaagaccgcccgaaatttgttttacatgacggacctccgtatgcaaacggcgacatccatatgggccatgcacttaacaagattttgaaagacttcattgtccgctataaatcaatgagcggctacaacgcaccgtatgtgccgggctgggatacacacggattgccaattgaaacagctctgacaaaaaacaaaaaggtcaaccgcaaagaaatgtcagtagcggaattccgcaaactatgcgaagagtacgcttggaagcaaatcgagggacagcgtgagcaattcaaacgtcttggtgtccgcggtgactgggaaaacccatatgtgacattaaaaccggaatacgaagcgcagcaaatccgcgtatttggtgaaatggcaaaacgaggctacatttacaaaggccttaaaccggttaactggtcaccttcaagtgagtctgctctggctgaagccgagatcgaatatcaagataaacgttcagcatctatttacgtcgcttttggtgtaaaagacggaaaaggcgttcttgaaaacggcgagcgcatcatcatttggacaacaacgccgtggacaattccggcgaacctcggaatctcagtgcaccctgatcttgagtacagcgtgattgcagtaggtgaagaccgctttgttgtagcaagtgccttagtcgaaaatgttgcatcggcatgcggatttgatcagtatgaagtgacaagaacggtcaaagggaaagaccttgagaacattatcgctgaacacccgctatatggcagagactctctcgttatgcttggtgaacacgtaacaactgatgccggaacaggctgtgttcatacagcgcctggacatggggaagatgactttatcatcggccaaaaatacggtttagatgtgctttgcccggtcgatgaaaaaggtgtaatgacaagcgaagctcctggctttgaaggcatgttctatgatgatgcaaacaaagcgatcacacagcagcttgatgaaaaaggcgcacttgtgaagcttgaattcattactcattcttatccgcatgattggagaacaaaaaaaccaaccattttcagagcaacagcgcaatggtttgcgtctattaaagatttcagatcagacctgctggatgccattaaagaaaccaaatgggttcctgaatggggcgagcagcgtttgcacaacatggttcgggaccgcggagactggtgtatttccagacagcgtgcgtggggtgtgccgattccggtattttacgctgaaaacggagaaccggttattacagatgaaaccattgaacatgtttctgaattgttcagacagcatggatcaaacatttggtttgaaaaagaagcaaaggatcttcttccggaaggctttacgcatcctggcagcccgaacggcacatttacaaaagaacaggatatcatggatgtttggtttgattcaggctcttcacatcaagcagtgcttgaagaacgtgatgacctcgttcgcccggctgatctatacctagagggatctgaccaatatcgcggctggtttaactcttctctttctacagcagtagccgtaacagggaaagcgccgtataaaggtgtgctcagccatgggttcgcactggatggagaaggacgtaagatgagtaaatcaatcggtaacgttgttgttccggctaaagtcatgaaacagcttggtgccgacatcttaagattatgggtatcttcagtggattatcaggcggacgttcgcgtgtctgacgccattctgaagcaggttgcggaagtatatcgtaaaatccgcaacacgttccgtttccttcacggcaaccttttcgattttgatccaaaaacaaatgcggtggctgtcgaagatcttcgcgaagtggatcagtatatgctgattaagctgaacaagctgattgataaagtgaaaaaagcgtatgatgagtacgaatttgcggttgtgtatcacagcattcataatttctgcacaatcgaattgagctcattctaccttgattttgcaaaagatattgtctacatcgagcatgcggatcatccggatagacgcagcatgcagacagtatcctacgaaacgcttcttgcattagtgaagctttcagcgcctatccttccacatacggcagacgaattgtggtctcatttaacatttgttgaagagcagagcgttcagttgaccgatatgccggaaacaatcacggttccaaacagtgaagcgactgaagaaaaatttgaccgctttatggctcttcgtgatgacgtgttaaaagcattagaaactgcgcgaaatgaaaaaattatcggtaaatctttggaagcaaacctgaaattgtatccaaacaaagaaaacaaggagctcttggcttccataaaagaaaacctttctcagctgtttattgtgtctgaactgacaatcagcgaagaaaatgaagcgccgaacgatgcgcaaagctttgcgacgggtaaaatcgctgtcgagaaagcggaaggcgaaatgtgtgagagatctcgtgtgatttcaaaagatgtaggggcaaatccgaaatatcctacactttcattacgcaacgctgaaatcgttgaaaaatactatcaaaaa

SEQ ID NO: 36 - DNA, 2412 bp, B. subtilis
  leucyl-tRNA synthetase
ttgagttttcagcacaaagagatagaaaagaaatggcagacatattggcttgaaaacaaaacatttgccactcttgataataatgaaaaacaaaaattttacgcgctggacatgtttccttatccgtctggagctgggttgcacgtcggccatcctgaaggatacacagctacggatattctgtcccgcatgaagcgcatgcagggctatgatgtccttcatccaatgggctgggacgcattcggcctgccagctgaacagtacgcgcttgacacagggaacgaccccgctgtgtttacgaagcagaatattgataacttccgccgccaaattcaagcgcttggcttctcatatgactgggatcgcgaaatcaatacgactgaccctgaatactataaatggacgcaatggattttcttaaagctatacgaaaaaggccttgcttacgttgacgaagtgcctgtaaactggtgccctgcgctcggtactgttcttgccaacgaagaagtcattgacggcaagagcgaacgcggcggccatccggtagagagacgcccaatgaagcagtggatgctgaaaatcaccgcttatgcggacaggctccttgaggacttggaagagcttgattggccggaaagcattaaagatatgcagcgcaactggatcggccgttcggaaggcgctcacgttcattttgctatagatggacatgatgattcctttacagtgtttacaacaagaccagatacgctgtttggcgctacatacactgtccttgccccggaacacgcattggtggaaaacatcacaacggcagagcaaaaagaagctgttgaagcttatatcaaagaaattcaatcaaagagtgacctagaacgcacagatcttgcgaaaacaaagacaggcgtatttacaggagcgtatgcgatcaatcctgtaaacggagaaaaactgccgatttggattgcggattatgttcttgcatcatacggaacaggtgctgtcatggcagttccaggacacgatgagcgtgattttgaattcgccaaaacattcggccttccggtgaaggaagtcgtaaaaggcgggaacgttgaggaagcagcctatactggcgacggcgagcacgtgaactctgatttcctgaacggccttcacaaacaggaagcgattgaaaaagtgatcgcttggctggaagaaacgaaaaacggtgagaagaaagtgacgtaccgtcttcgtgactggctcttcagccgccagcgttattggggcgagccgattccggtcattcattgggaagacggaacgtcaacagctgtcccggaagaggagctgccgctgattttgccaaaaacggatgaaatcaaaccgagcggaacgggcgaatcaccgcttgcgaacattaaagagtgggtggaagtcacagaccctgagacagggaaaaaaggaagaagagaaacgaatacaatgccgcaatgggcgggaagctgctggtatttcttgcgctatattgatccgcacaatccggatcagctggcatcaccagaaaaattggaaaaatggcttccggtcgatatgtatatcggcggtgcagaacatgccgtgcttcaccttctgtatgcccgcttctggcataagttcctttatgatatcggcgtagtgccgacgaaagaaccgttccaaaagctgtacaaccaaggaatgattctcggcgaaaacaacgaaaaaatgagtaaatctaaagggaacgttgtcaatcctgacgaaatcgtggcctctcacggtgctgatacgctgagattgtacgaaatgttcatgggacctcttgatgcttcaatcgcctggtctgaatcaggattagacggtgcgcgccgtttccttgaccgtgtatggcgcctatttattgaagacagcggtgagcttaatggaaaaatcgttgaaggcgcgggtgaaacattggagcgcgtctatcatgaaacggtcatgaaagtcacagaccattatgaaggccttcgtttcaacacgggtatttcccagctgatggtctttattaatgaagcttataaagcaacagaactgccgaaagaatatatggaaggcttcgtgaagcttctttctcctgtcgcgccacacttagcggaagagctatgggagaagcttggccattccggcacaattgcctacgaagcttggcctgtatatgatgaaacaaaacttgtggatgatgaagttgaaatcgttgttcagctgaacggaaaagtaaaagcgaaattacaggttcctgccgatgcaacgaaagaacagctggaacagcttgctcaagcagatgaaaaggtcaaagagcagcttgaaggcaaaacgattcggaaaatcatcgcggtgcctgggaagcttgtcaatattgtggcaaac

SEQ ID NO: 37 - DNA, 1497 bp, B. subtilis
  lysine tRNA synthetase
atgagtcaagaagagcataaccatgaagaattgaatgatcagctgcaagtcagacgcgataaaatgaatcagctgagagataacggcatcgatccattcggcgcacgttttgaaagaactcatcagtctcaagaagttatttcggcatatcaagatctaaccaaagaagagttagaagaaaaagcgattgaagttacaatcgcaggccgcatgatgacaaaacgcggcaaaggaaaagccggctttgcccatcttcaggatttagaaggccaaatccaaatctacgtaagaaaagacagtgtcggtgacgaccaatatgaaatcttcaagtcttctgacctcggtgatcttatcggcgtaaccggaaaagtcttcaaaacaaatgtaggcgaattgtctgttaaagcaacttcctttgaattgctgacaaaagcgcttcgtccgcttcctgacaaataccatggtttaaaagacgttgagcagcgctaccgtcagcgctatctggatcttatcgtaaacccagacagcaaacatacgttcattacacgaagcaagatcattcaagctatgagaaggtaccttgatgatcatggatacttagaagtagaaacacctacaatgcacagcattcctgggggagcttctgcacgtccgtttatcactcaccacaacgcgttagacattccactctatatgcgtattgctatcgaactgcacctaaaacgtctaattgtcggcggtttagaaaaagtatatgaaatcggccgtgttttccgtaacgaaggtgtctctacacgccataaccctgaatttacaatgattgagttatatgaagcatatgcggactataaagatatcatgagcttaactgaaaaccttgtcgctcatatcgcccaagaagtgcttggcacaactacgattcaatacggggaagagcaaatcgaccttaaaccggagtggaaaagaatccatatggttgacgcagtcaaagaagcgaccggcgttgatttctgggaagaggttactgttgagcaggcgcgtgaatatgcaaaagaacatgaagtagaaattaaagactctatgacagtaggccatatcatcaacgaattcttcgaacaaaaaattgaagaaacgcttattcagccaacgtttatttacgggcatcctgtagaaatttctcctcttgctaagaaaaaccctgaggacccgcgttttacagaccgttttgagctgtttatcgttggccgtgaacatgccaacgcgtttacagagctgaatgatcctattgatcaaagagaacgctttgaagcgcaattaaaagagcgtgaagccggtaatgatgaagctcatttaatggatgaagactttgttgaagctctggaatacggtatgccgccaacaggaggtttaggcatcggtatcgaccgtctggttatgctgctgacgaatgccccttctattcgcgatgtgctgttattcccgcaaatgagacaacgc

SEQ ID NO: 38 - DNA, 1176 bp, B. subtilis
  histidyl-tRNA synthetase, hisZ
atgtttatgtttgaaaaaccgcacggcatgagagatacactgcccggtttatacgaaacgaaaaaaaaggtgagacgatcgttaaccgatttgattgataaatggggatatcaatttatggaaacgccgacactggagttttacgataccgttggcgtccagtcagcaattgaagagcagcagctgtttaagctacttgatcaggacggcaagacattggtgcttcgcccggatatgacggggccgattgcaagggtggcggcatcgaagcttctgaaacacggtcatccgctaagagtcggctatgcggcaaatgtattcagggctcaggagcgtgaaggcggacgtccggctgagttcgagcaggtcggagtggagttaatcggtgacggcacgacgagcgcggatgcggaggtcattgctttagtcgtcggggcattaaaaaacgctgggctggcatcctttaaaattgcaattggccatgccggcattgcggatgctttgtttgttgaggtgctcggaaacgttgaacgagctgatgtgctgcggaggttcttatatgaaaagaactacgtcggctacagagagcatgtcaagtctctcccgctttcctccattgataaaagcaggctgcttgagctccttgaactgcggggcggtatagaagtatgcggacgtgccgaggaaatcgtcgattctgcgcaaggaaaaagcgtggttgatgagctgaaggcgctgtgggacattcttgaggattacggatgtacggaaaatgtccgcctggatctgaatatggtcagccacatgagctattacacagggattttatttgaagtgtacgccgagaatgtcggttttgtcattggaagcggcggccgttacaacaagctgctgggccattttgattcacctgcaccggcaacaggcttcgggcttcggatcgaccggctgattgaagcccttcatatgaaggacgaaccttgtgaaatagacgctgttattttcagcaaagagcagcgggcgcaagccatcgcttatgcgaatgaagaacgcatgaaagggaacaaagtggttcttcaagatttatcgggaatagaaaatatcgaccagatgacaaaatcttttgcaaacgtcacttattttatcggtgccagaaaggaagagcaaaatgggtaa

SEQ ID NO: 39 - DNA, 1992 bp, B. subtilis
  methionyl-tRNA synthetase
atgccgcaagaaaacaatacattttacattacaacaccgatttattatccgagcggaaaattacatatcggccatgcatatacgacagtcgcaggagatgcaatggcacgttacaaaagattaaaagggttcgatgttcgctatttaacgggaacggacgagcatggacaaaagatccagcaaaaagctgaacaggaaaacattacacctcaggagtatgtggatcgcgcagcggcagatattcaaaaactgtggaagcagcttgaaatctcaaacgacgactttatccgcacgacagaaaaacggcataaagttgtcattgaaaaagtgtttcaaaagcttcttgataacggggacatctatcttgatgagtatgaaggctggtacagcatccctgatgaaacgttctacacggaaactcagctcgttgatattgagcggaatgaaaagggagaggtcatcggcggaaaaagccctgacagcggtcacccagttgaattgatcaaagaggaatcttatttcttccgcatggggaaatacgcggatcgtcttctgaaatactatgaagaaaacccgacattcattcagccagaatcacgcaaaaacgaaatgattaataactttatcaaacctggacttgaggatttggcagtatcacgtaccacttttgattggggcgtgaaggtgccggaaaatccaaagcatgttgtatatgtttggattgacgcactatttaactatttaacggcactcggttatgatacagaaaatgatgagctttatcaaaaatattggcctgccgatgttcatttagtcggtaaggagattgtacgattccatactatttactggccaattatgctgatggcgctggatcttccgctgccaaagcaagtattcgcgcatggctggcttttgatgaaagacggaaaaatgtcgaaatcaaaaggaaacgttgtagatccggttacattaattgaacgctatggtttagacgaacttcgctattacttgcttcgcgaagtgccgttcggatcagacggtgttttcacgccggaaggatttgttgagcgaatcaactatgatttagcgaatgatttaggaaatctattgaatcgtactgttgcgatgattaataagtattttgacggacaaatcggttcttacaaaggtgctgttacggaatttgaccatacgctcacttctgttgctgaagaaacagtgaaagcttatgagaaagcaatggaaaatatggagttctcggtagcactttcgactttatggcagctcatcagccgcacaaacaaatacattgatgagacagctccatgggtgcttgcgaaagatccggcaaaagaagaagaattgcgatcagttatgtatcatctcgctgaatcattgcgtatttcagcggtactgcttcagccattcttaacaaaaacacctgaaaaaatgttcgagcagctgggcattactgatgaatctttaaaagcttgggacagcattacagctttcggccagctgaaagatacaaaagtacaaaaaggcgagccattgttccctcgtttagaggcagaagaagaaattgcttacatcaaagggaaaatgcaaggttcagcaccagcgaaagaagaaacaaaagaagaagagcctcaagaggtcgatcgtttacctgaaattacgattgatcaatttatggatgtagagcttcgcgtagctgaggtcattgaggcagagccagtgaaaaaagcagaccgtttattgaagctgcagcttgatcttggttttgaaaagcgccaagtggtatccggcattgcgaagcattatacgcctgaagagcttgttggaaaaaaactcgtttgtgtaacaaatctaaaaccggttaaactcagaggagagctttctcaaggtatgatccttgcaggagaagcagacggcgttttaaaggtcgtatctatcgatcagtcgttaccaaaaggcacaagaattaaa

SEQ ID NO: 40 - DNA, 1032 bp, B. subtilis
  phenylalanyl-tRNA synthetase alpha subunit
atggaagaaaagctaaaacagctggaacaagaagctttagaacaagtagaagcggcaagctcattgaaggttgtcaatgatattcgggtgcaatatctcggaaaaaaagggccgattacagaagtgctgcgcggaatgggcaagctttctgctgaggaacgtccaaaaatgggggcgctcgcgaacgaagtaagggagcgtattgccaatgcgattgctgacaaaaacgagaagcttgaagaagaggaaatgaaacagaagcttgcaggacagacaattgacgtcacgctgccggggaaccctgttgcagtcggcggccgccatccgctcactgttgtcattgaagaaattgaagatttatttatcggtatgggctacacagtcgaggaagggccagaggttgaaacggattactacaacttcgaatcgctcaatcttccgaaagaacacccagcgcgcgatatgcaggacagcttttacatcacagaggaaactttgatgagaacgcaaacttctcctgtccaaacacgtacgatggaaaagcatgaaggcaaaggtcccgttaaaatcatttgcccgggtaaagtatatcgccgtgataacgatgatgcgacgcactctcaccaatttatgcaaattgaagggcttgtcgttgacaaaaacatcagcatgagtgatttaaaaggaacgcttgaacttgttgcgaaaaaaatgttcgggcaagaccgtgaaatcagactccgcccaagcttcttcccgtttactgagccttcagtagaagtggatgtgacatgctttaaatgcggtgggaacggctgctcagtatgtaaaggaacaggctggattgaaatcctcggtgccggaatggttcacccgaacgtgcttaaaatggctggctttgatccgaaggaatatcagggcttcgcattcggaatgggtgttgagcgcatcgcgatgctgaaatatggcattgatgatatccgccacttctatacaaacgatgtcagatttatttcgcagtttaaacaggcg

SEQ ID NO: 41 - DNA, 2412 bp, B. subtilis
  phenylalanine tRNA synthetase, beta subunit
atgtttgtttcttataaatggttagaggattatgttgatttaaaaggcatggacccggctgttcttgctgaaaaaattacaagagccggtattgaggttgaaggaattgaatacaaaggagaaggcatcaaaggcgttgtcatcggccatgtgctggagcgcgagcagcacccgaatgctgataagctgaataagtgccttgtggatatcggagctgaagcccctgtacaaatcatttgcggcgcgccgaatgtggataagggacaaaaagtcgcagttgcaacagtcggagcggtgctgccgggcaatttcaaaatcaaaaaagccaagcttcgcggtgaagaatcaaacggcatgatctgttccttacaggagcttggtatcgaaagcaaacttgtggcgaaggagtatgcagaaggcattttcgtattcccgaatgacgctgaaacaggaagcgatgccttagcggctttacagcttgacgatgcgattcttgagctgggtttaacgccaaaccgcgcggatgccatgaacatgcttggtgttgcttacgaggttgcggcgattttagacactgaggtaaagcttccccaaacggattacccggctgcttcagaacaagcgtctgattacatttcagtcaaaattgaagatcaagaagcgaacccgctgtacactgcaaaaatcattaaaaatgtcacgattgctccgtcaccgctttggatgcagacaaagctgatgaatgcaggcattcgtccgcacaacaacgtcgttgacatcacaaattttgtcctgttggaatacggacagccgcttcatgcgtttgattatgacagatttggctctaaggaagtcgtagtaagaaaagcagctgagaatgaaatgatcgtaacacttgatgatcaagaacgtaagctgtctgccgatcaccttgtcattacaaacggaacgaaagcgcaggccgttgccggtgtcatgggaggagcagagtcagaggttcaggaagatacgaaaaccattttgcttgaggctgcgtatttcaacgggcagaaggttcgcaaggcttccaaagaccttggtttgagaagtgaatcaagcgtaagatttgaaaaaggaattgatccggcacgcgtacgtcttgcagcagaacgggctgcgcagctgatccatctgtatgccggcggtgaggtgcttgctggaacggttgaggaagatcacctgacaatcgaagcaaataacattcacgtatctgctgacaaagtgagcagcgttctaggcctgacgatcagtaaagaagaactgatcagcatttataaacgtctcggttttacggtcggtgaagcggatgatcttctcgttgtgactgtcccatcacggcgcggtgacattacaatagaagaggatttaatcgaagaagcggcaagactgtacggctacgataacattccgtctacgcttcctgagacagcgggaactacaggcggattaacgccatatcaggcaaaacgccgtaaagtcagacgcttccttgaaggcgcaggcttatcacaagccatcacttactcactgacaaacgagaagaaagcgacagcgtttgcgattgaaaaatcactgaatactgtgcttgcgcttccaatgagtgaagaaagaagcattctcagacacagtcttgttccaaatctgcttgattctgtttcttacaatcttgcgaggcagactgactcagttgcactgtacgaagttggctccgttttcctaacgaaggaagaggacacaaaacctgttgaaacagaacgcgtagcaggagctgttaccggattatggcgcaagcagctatggcagggtgaaaagaaaccagttgatttctttgttgtcaaaggaattgtggaagggctgttagacaaactcaacgtcctagacagcattgaatttgtacagtctgaacgcaaacagctgcatccgggcagaacagcgaacattttattgaatggttctctgatcggcttcatcggccaagtccatccttcattggaaaaagaactggatataaaggaaacatacgtatttgagctcgatcttcatgcgttgctcgcagcagaaacagcaccgcttgtttacacggcgattccaaaatatccgtctgtgacacgtgatatcgctcttgtaacagataaaaccgtcacaagcggacagctcgagagcgtcattaaagaggccgggggcaagcttctgaaagaggtaaccgtatttgacgtatacgaaggagagcatatggaggaaggcaagaagtcagtcgctttctcactccagtatgtcaatcctgaacaaacactgactgaagaagaagtgacaaaggcgcacagcaaagtgctgaaagcgttagaagacacatatcaagctgttttaagaggc

SEQ ID NO: 42 - DNA, 606 bp, B. subtilis
  similar to phenylalanyl-tRNA synthetase, ytpR
atgaacgctttttataataaagaaggtgtgggagacacgctcctgatctctcttcaagatgtgacacgcgaacaattaggctatgaaaaacacggcgacgtcgtcaaaattttcaataacgaaacaaaagaaacaacgggcttcaacattttcaatgcgtcttcttacttgaccattgatgaaaacggccctgttgcgctctcagaaacattcgtgcaagatgtgaatgagattttaaacagaaacggcgttgaagaaacattagtcgttgatttatctccgaagtttgttgtgggttatgtagaatcaaaagaaaaacatccgaatgcggataaattaagcgtatgtaaagtcaacgtaggagaagaaacgcttcagatcgtctgcggcgcgcctaacgttgaccaaggccaaaaagttgtcgttgccaaagtcggtgctgtgatgcctagcggacttgtcattaaggatgcagagcttcgtggcgttccgtcaagcgggatgatctgctcagcgaaagagcttgatttgccggatgcccctgctgaaaaaggaatccttgtgttagaaggagactatgaggcgggagacgcatttcagttttag

SEQ ID NO: 43 - DNA, 1692 bp, B. subtilis
  prolyl-tRNA synthetase
atgagacaaagcttgacgcttattcctacgctccgtgaagttccagctgatgccgaagcaaaaagtcatcagcttcttctgagagcaggatttatcagacagaatacgagcggggtatacagctatatgcctcttgcgtataaggtgattcaaaacattcagcagattgttcgagaggaaatggagaaaattgatgccgtagaaatgctcatgcccgcattgcagcaggcagagacatggcaggaatcaggcagatggtatacgtatggtcccgaactgatgagactaaaagaccgtcatggccgtgaatttgctttaggggcaacgcatgaagaggttatcacttcgcttgttcgcgatgaggttaaatcttataagcgtctccctctgactctttatcaaattcagtctaagttcagagatgaaaaacgtcctcgcttcggtttgttaagaggccgcgaatttattatgaaggatgcgtactctttccatgcatctgcagagagcctggatgaaacgtatcaaaaaatgtacgaggcctattctaatatttttgcccgctgcggcattaatgtaagacctgttatcgctgattcaggcgcaatgggaggaaaggatacgcacgaatttatggcactttctgcgatcggagaggatacgattgcgtattctgatgaatcacagtacgcggctaatatcgagatggctgaagttcttcaccaggaagttccttcagatgaagagcctaaagctctagagaaggttcatacgcctaacgtgaaaacaatcgaagaactgactgcgttcttacaggtttcggctgaagcgtgcattaagtcagtattgtttaaagctgatgaccgttttgtcttagtgcttgtaagaggggatcatgaagttaacgatattaaagtgaaaaacttgcttcatgcagaagttgtagagcttgctacacatgaagaggtcattcagcagctcggaacagagccaggctttgtaggccctgtcggtattcatcaggatgtggaagtatatgccgatcaagctgtgaaagcaatggttaatgctgttgccggggcaaatgaaggagatcatcattataaaaatgtcaatgtgaatcgtgacgcgcaaattaaagaatttgctgatcttcgttttattaaagaaggtgatccttcaccagacggcaagggaacgatccgttttgctgagggaatcgaagtcggacaagtctttaagctcggaacacgctattcagaagcgatgaatgcgacatatttagatgaaaacggacgcgcgcagccaatgctgatgggctgttacggaatcggtgtgtcaagaacgctttctgctattgctgaacagcatcacgacgaaaaaggcttaatatggccaaaaagcgttgcgccgtacgatcttcatattcttgctttgaacatgaaaaacgatgggcaaagagagcttgctgaaaagctgtatgccgatttaaaagcggaaggctatgaagtgctctatgatgaccgtgctgagcgtgccggcgtaaaattcgctgattcagatttaatcggccttccaatccgcatcactgtcggaaaacgagctgacgaaggaatcgtcgaagtgaaaattcgtcaaactggtgagtcaactgagatttcagtagacgaattatctgcgtttatcagcaagcag

SEQ ID NO: 44 - DNA, 1275 bp, B. subtilis
  seryl-tRNA synthetase,
atgcttgatacgaaaatgctgagagcaaattttcaagaaattaaagcaaagcttgtacacaaaggcgaagacttaactgattttgataagtttgaggcgctggatgatagacgaagagagcttatcggcaaggttgaagagttaaaaggaaaacgaaatgaagtttctcagcaggttgctgtgctgaagcgtgagaaaaaagacgcggatcacattattaaagaaatgcgtgaagtcggcgaggaaattaaaaagctcgatgaagaattacggacagtggaagctgagcttgatacaatcctgctctcaatcccgaatattccgcatgagtctgtacctgtcggtgaaacagaagacgacaacgtagaagtgcgtaaatggggtgaaaagccttcatttgcttatgagccgaagccgcactgggatattgcggatgagctgggtattctggattttgaacgtgctgccaaagtaacaggaagccgtttcgtgttctataaaggcttaggtgctcgtctggagcgtgcgctttataactttatgcttgatctgcatgtggatgagtataactacactgaagtgatcccgccatatatggtaaaccgcgcaagcatgacgggaacggggcagcttcctaaatttgaagaggatgcatttaaaatcagagaagaagattatttcttaattccaacggcggaagtgccgattacaaacatgcatcgcgatgaaatcctttcaggtgacagcctgccgatcaactatgcggcattcagtgcttgcttccgttctgaagctggttcagcaggacgtgacacacgcggattaattcgtcagcaccaatttaataaagttgagcttgtaaagtttgtgaagcctgaagattcttatgaagaattagagaaacttacaaaccaagcagaacgggttcttcagctgcttgagcttccataccgtgtcatgagcatgtgtacgggtgacttaggctttacggctgcgaaaaaatacgatatcgaagtttggattccaagccaagacacatatcgagaaatctcttcttgcagcaacttcgaagcgttccaggcgagacgtgcgaatattcgtttcagacgtgaagcgaaaggcaagcctgagcatgtacacacactgaacggctcaggactggcggttggaagaacagttgctgctatcttagaaaattatcagcaggaagacggaagcgttgtgattccgaaagtgcttcgtccttatatggggaatagagaagtaatgaaaccg

SEQ ID NO: 45 - DNA, 1929 bp, B. subtilis
  threonyl-tRNA synthetase, major
atgtcagatatggtaaaaatcacatttcctgatggagcagtcaaggagtttgcgaaaggaacaacaacagaagatatcgcggcatccatcagtccgggattaaagaaaaagtcattagccggaaaactgaacggaaaagaaatcgatttgagaacgccgatcaatgaagacggtacagtggaaatcattacagaaggctcagaagaaggtcttcaaattatgcgccacagtgcggctcacctgctggctcaagcgattaaacgcatctacaaggatgttaaattcggcgtcggtccggttatcgaaaacggtttttactacgatgtagaaatggacgaagcgattacaccggaggatctgccgaaaatcgagaaagaaatgaaaaaaatcgttaatgcgaaccttccgatcgttcgaaaagaagtcagccgtgaagaagcgaaagcccgttttgcggaaatcggcgacgacctgaagcttgaactattggatgcgattcctgaaggagaaaccgtttcgatctatgagcaaggcgaattctttgacctgtgccgcggcgtccatgttccttcaaccggaaaaatcaaagaatttaagctgttaagccttgcaggcgcatactggcgcggtgacagcaaaaaccaaatgcttcagcgcgtatatggtacagctttcttcaaaaaagctgatcttgaagagcatcttcgtatgctagaagaagcgaaagaacgcgaccacagaaagcttggcaaagaattaaagctgttcgcgaactctcaaaaagtcggacaaggcctgccgctttggctgccaaaaggcgcaacaatccgccgcgtcatcgagcgctacattgtcgataaagaaatcagcctcggctatgagcacgtatacacacctgtgctaggcagcaaagagctgtatgaaacatcaggacactgggatcattatcaggaaggcatgttccctccgatggaaatggacaatgaaacacttgtattgcgtccgatgaactgtccgcaccatatgatgatttacaaacaagacattcacagttaccgcgagcttccgattcgtattgcagagcttggaacgatgcaccgctatgaaatgtcaggtgcgctgtcaggacttcaacgggtacgcggaatgacgttaaatgatgcacacatctttgtgcgcccggatcaaattaaggatgagtttatccgtacagtccgtttaatccaggatgtttatgaagacttcggtttaagtgattacacattccgtctgtcttaccgcgatccggaagatacagagaaatattttgatgacgatgaaatgtggaacaaagcgcaatccatgctgaaagaggcaatggatgaaatcggccacgactattacgaagcagaaggtgaagcggcattctacggacctaaacttgatgttcaggtgaaaactgcgatcggaaaagaagaaacactgtctacggttcagcttgatttcttattgccggaacgtttcgacctgacatacatcggtgaggacggcaagcagcaccgtccggttgtcattcacagaggtgtcgtttcaacaatggaacgctttgttgctttcttaatcgaagaacacaaaggcgcgctgccgacatggcttgcaccggttcagttccaagtcatcccggtttctccggctgtgcatttagactacgcgaaaaaagtgcaggaacgcctgcaatgtgaaggcctgcgtgttgaagtcgacagccgcgatgaaaaaatcggctacaaaatccgtgaagcgcaaatgcaaaaaatcccgtacatgctggtggtcggtgaccaagaagcagaaaacggagcggtaaacgtgcgtaaatacggagaacagaactctgaaaccatttcacttgatgagtttgtgaaaaaggcagtagctgaagcgaaaaaa

SEQ ID NO: 46 - DNA, 1914 bp, B. subtilis
  threonyl-tRNA synthetase, minor
atgagcaaacatgtacacattcagcttccggacggacagatccaggaatatccgaaaggcatcacaatcaaagaagcggctggttcgatcagttccagtctgcaaaagaaggcggccgcaggtcaggtcaacggcaagcttgttgatctcagtttcaagctggaggaggatgctgagctttcgattgtcacactcgattcacaagaaggcttgcaggtcgttcgccacacaacagcgcacgtgctggcccaagcagtaaaacggctgtacggcgaagtgtcattgggggtagggcctgtgattttagacggcttctactatgacatgaagctggggaaaagcttggcatcaggagacttagaggccatcgaaaaagaaatgaaaaacataataaatgagaaccttgaaatcaaacgtatagaggtttcttacgaagaagctgaagagctgtttgcacaaaaagacgagcggctcaagcttgagattttaaaggatattccgcgcggggaggatatcacactgtaccagcaaggggaattcgttgacctgtgccgagggccgcatcttccttctacagggatgatcaaggcgttcaaactgacaagagtatcaggcgcttattggcgcggagacagcaaaaatgaagtcctgcagcgggtgtacggggttgcgtttcaaaagaaaaaagatttggatgcgcacctgcatatgctagaagaagcggcaaaacgagaccatcgcaagctcggcaaacagttagggctgtttatgttttctgaagaagcgccggggatgccgttttatttgccgaaagggcagattgtccgcaatgagctggagcgcttctcgcgtgagctgcaaacaaatgccggctacgatgaggtgcgtacgccgtttatgatgaatcagcggttatgggagcagtcaggccattgggaccattatcgcgacaatatgtacttttcagaagtggatgatacaagattcgcaatgaagccgatgaattgccccggccacatgctcatctttaaaaacagcctgtattcttatcgggatctgccgattcgcatggcggaattcggacaggtgcaccgccatgaatacagcggcgctttaaacggaatgcttcgcgttcggacgttctgtcaggatgacgcccatattttcgttcgggaggatcagattgagagtgagatcaaagaagcgattcggctgattgatgaggtatatcggacatttggtttcgagtattctgttgagctttcgactcgcccggaggattcattaggcgatgacagcctttgggaagcgtcggaacgtgccttagcacgcgtccttgaggagcttggcctctcttatgagatcaatgaaggcgatggggcgttttacgggccgaaaattgatttccatataaaagatgccctgaaacggagccaccaatgcgcgacaattcagcttgattttcaaatgccggaaaaatttgatttaacatatatcaacgaactcaatgagaaggttcgtcccgttgtcatccacagagcggttttcggctcaatcgaccgtttcttcggcattttgatcgagcattacggtggggcttttccagtctggctcgcaccgatccaggttcaaatcataccggtttcgcacgttcacttagactattgcagaaaagtgcaggcggagctgaagcaggcggggataagagccggcattgatgagcgcaatgagaagctcggctataaaatcagagaatcgcaggtgcaaaaaattccgtacgtgctggtgcttggagatcatgaggagcaggagaatgcggtaaacgtccgccgattcggacatcagcagaatgaacacgttcctttccaaacttttaaagataaacttgtgaaacaggtggaaaatcgaggcatg

SEQ ID NO: 47 - DNA, 990 bp, B. subtilis
  tryptophanyl-tRNA synthetase
atgaaacaaacgattttttcaggcattcagccaagcggctcagtgacgctcggcaactatatcggtgcaatgaagcagtttgtcgaactgcagcatgattataacagctatttttgcatcgtcgatcagcatgcgataactgttcctcaagaccggcttgagcttagaaagaatatccgcaatctcgcggcgctttacttagctgtcggacttgatccagaaaaagcaacattgtttattcagtcagaggtccccgcacatgcgcaggccggatggatgatgcagtgtgtcgcctatatcggcgagcttgagcggatgactcaatttaaggacaaatccaaaggcaatgaagctgtcgtctccggcctgttaacatatccgccgctgatggccgctgatattctgctgtacggaacggatcttgtacctgtcggcgaggatcaaaagcagcaccttgagctgacgcggaatcttgcagaacgcttcaacaaaaaatacaacgacatctttacgattccggaagtgaaaattccaaaagtcggtgcacgtatcatgtctctgaatgatccgctgaagaaaatgagcaaatctgatccgaatcagaaagcttatattacattgctggatgagccgaagcagcttgaaaagaaaatcaaaagcgcagtaacggattctgaaggcattgtcaaatttgataaggaaaacaaaccgggcgtttccaaccttcttacaatttattcaatcctcggcaatacgacaattgaagagcttgaagcaaagtacgaaggaaaaggctacggcgagtttaaaggtgatttggcagaagtcgtagtgaacgcattaaaaccgatccaggaccgctattacgagctgatagaatctgaagaattagaccggattcttgatgaaggcgcggaacgagcgaatcggacagcaaacaaaatgctgaaaaaaatggagaatgccatgggtcttggaagaaaaagacgc

SEQ ID NO: 48 - DNA, 1266 bp, B. subtilis
  tyrosyl-tRNA synthetase, major
atgactaacttacttgaagacttatccttccgcggattgattcagcaaatgacagatgaagaagggttaaataaacagctgaatgaagaaaaaatccgcttgtactcaggctttgaccctacagcagacagcttgcatatcggacacctgctgcctattttaacacttcgccgttttcagcttgcagggcatcatccgattgcgcttgtgggcggcgcgacgggcctcatcggtgatccaagcggaaaaaaagcagagcgtacattaaacactgctgacatcgtatccgaatggtcccaaaaaatcaaaaaccagctgtccagatttctagattttgaagcagcagaaaacccagctgtcatcgcgaacaactttgactggatcggcaaaatgaatgtgatcgatttccttcgtgacgttggtaaaaatttcggcatcaattacatgctggcaaaagatacggtcagctcaagaattgaatccggcatctcgtacacggaattcagctacatgattcttcaatcttatgatttcttaaatctttacagagacaaaaactgtaaactgcaaatcggcggaagcgatcagtggggcaacatcacagcgggtcttgaactgatcagaaaatcagaagaagaaggagcaaaagcgtttggcctaacgattccgcttgtcacaaaagcagacggcacgaagttcggaaaaacggaaggcggcgcgatctggcttgataaagaaaaaacatcgccatatgaattctatcaattctggatcaacacagacgaccgtgacgttgtcaaatacttgaaatacttcacgttcctttcaaaagaggaaattgaagcatacgctgaaaaaacagaaacggcgccagaaaaacgtgaagcgcaaaaacgcctggcagaagaagtgacatcacttgttcacggacgcgaggcgctggagcaagccatcaacatctcccaagcgctgttcagcggcaatattaaagagctttctgctcaagacgtaaaagtaggctttaaagatgttccttctatggaagttgacagcacccaagagctttcacttgtagatgtgttggtgcaatctaaattatctccttctaaacgccaagcgcgtgaagacatccaaaacggagctgtttacattaacggtgaacgccagacggaaataaattataccctatcaggtgaagaccgcatcgaaaaccaatttactgttctgcgccgcgggaagaaaaaatacttccttgtgacgtataaa

SEQ ID NO: 49 - DNA, 1239 bp, B. subtilis
  tyrosyl-tRNA synthetase, minor
atgatgagaacatttgagcagctcacagcgtcacaacaaaaagaggtagaaagacagcttcagctatacatgacaggcgcccacgaagtcataccgccggaggaattaaaggcaaagctcgtgaaatcaatttccacgggcacgccgcttaaaattaagctcggattagatccgtctgcaccggatgtacatttgggccatacggttgtgttaaataagcttcgccaatttcaagaaaacggccacattgtccagctgttaattggggatttcacaggaaaaattggtgatccgaccggaaaatcggcagccagaaagcaactgactgatgaagaagttcagcacaatgccaaaacctactttgagcaattcggaaaagtgcttgatccagaaaaagtcgagcttcactataactcaaaatggctgaaaacattgaatctagaagatgtcattgaattagcagggaaaataacggtagcccgcctgatggagcgcgacgactttgaagaacgcatcgccatgcaaaaaccaatctcactgcatgaattcttttacccattgatgcagggctatgattctgtcgttctcgaaagtgatattgaattaggcggaacggatcagcatttcaatgtcctcatgggacggcatttccaagaacgatacaacaaagaaaaacaagtcgtcatccttatgccgctcttggaaggcttggatggcgtcgagaaaatgtcgaagtcgaaacacaactacattggcattaacgaacatccaaacgacatgtacggaaaaacgatgtcactgcccgacagcctgatgaaaaagtacatccacttggcgacagacttagagcttgaagagaaaaaacagctcgtaaaagacttagaaaccggcgccgttcatccgcgtgatgccaaaatgcttttagccagaacaatcgtccgaatgtatcacggagagaaagcagcagaagctgccgaacactcgtttaaaacagtctttcaggaaaacagcctgccggaagatataccggccgtaaactggaaaggcgaaaaaacgatagcgatgattgatctgctcgtcaagctaaagctcctctcttcgaagagcgaggcgcgccgcatgattcaaaacggaggtgtacgcatcgacggagagaaagtaacagatgttcacgccaaagcagagataagagagaatatgatcatccaagtcggcaaacgcaagtttttaaagctccaa

SEQ ID NO: 50 - DNA, 2640 bp, B. subtilis
  valyl-tRNA synthetase
atggaaacgaatgaacaaacaatgccgacgaaatatgatccggcagcggttgaaaaagaccgatatgatttttggctaaaaggcaaattttttgaagcgggcagtgaccagacaaaagagccatactctgttgtcatcccgccgccaaacgtaacagggagacttcaccttggccacgcatgggacacaacgctgcaagacattgtaacaagaatgaaacgcatgcagggctatgatgtgctgtggcttcctggcatggaccacgccggcatcgcgacacaggctaaagtggaggcgaaacttcgtgaagaaggcaaatcccgctacgatttaggccgtgaaaaattcctcgaagaaacgtggaagtggaaggaagaatatgctgactttattcgcagccagtgggcgaaattaggtcttggcctagattattcccgtgaacgttttacgctggatgaaggcttgagcaaagcggtaagggaagtattcgttaagctttatgaaaaaggcctgatctacagaggcgaatatattattaactgggacccagcgacaaaaacagccctatccgacattgaggtgatttacaaggatgttcaaggcgcattctatcatatgagctatccgcttgctgacggttcaggctcaattgaaattgcgacgacacgtcctgaaacaatgctcggtgatacagctgttgccgttcaccctgaagacgagcgctataaacaccttatcggaaaaacggtgatcctgccgattgtaaatcgtgaaattccgattgtcggtgatgactatgttgatatggaatttggttcaggtgccgtgaaaattacgcctgctcatgatccgaacgacttcgagcttggcaaccgccacaatttagaacgcatccttgtcatgaatgaagatggcacgatgaatgaaaatgcgctccaatatcaaggtatggatcgttttgaatgccgcaaaaagctcgtgaaagatttacaggaggcgggagttctcttcaagatcgaggatcatatgcactctgtcggccatagtgaacgaagcggggctgttgtggagccttatctttctacacaatggtttgtgcgcatgcagccgcttgctgatgcagcgattgagctcgaaaaaaaagaagaaaaggtcaactttgtgcctgaccgtttcgaaaaaacgtatttacactggatggaaaatatccgcgactggtgtatctcccgtcaattgtggtggggccatcgtattcctgcctggtatcataaagaaacaggcgagctatatgtcggacttgaagcgccggaagacagcgaaaattgggaacaggatacagacgtactggatacatggttcagttctgcgctatggcctttctccacaatgggctggcctgatgtaacggctgaagactttaaacgctactatccgacagatgttctcgtgacagggtacgatatcattttcttctgggtatcacgcatgattttccaaggcattgaatttacaggcgagcgcccgttcaaagatgttttaatccacggcttaatccgtgacgagcaaggccggaaaatgagtaaatctcttggcaacggtgtcgacccgatggatgtgattgacaagtacggagccgattctctgcgttatttccttgcgactggaagctctcctggccaggatctgcgcttcagctatgaaaaagtggaatcgacctggaattttgccaataaaatttggaacgcctcccgttttgcattaatgaatatggatggcatgacgtacgatgagcttgatctgtcaggtgaaaagtcagtcgctgacaagtggattttaacgcgattaaatgaaacgatcgagcacgtgacccagcttgctgaccgatatgaatttggtgaagtaggacgccatttatataactttatttgggatgatttctgtgattggtatattgaaatggcgaagcttccgctttatggagaagacgaagcggctaagaaaactacccgttccatccttgcttacgtacttgatcaaacgatgcgtctgctccatccgtttatgccattcttaacggaggaaatttggcagcaccttcctcaccaaggggaatcgattacagtaagtcaatggcctgcagtagtgccggagcataccgatactgaagcggcagctgacatgaagcttcttgttgagctgatccgttctgtgcgcaatatccgcagtgaagtcaatacgccaatgagcaagcaggttgaactgtacattaaaacaagcacagacgaaatagcgtcccgcctcgaagcgaaccgttcatatgttgaacgctttacgaatccgagcgtgcttaaaatcggcaccgatattgaggctgttgataaggcgatgacggccgttgtctcaggagcagaggttattcttccgcttgaaggcttaatcaatattgatgaagaaattgcccgtctgcaaaaagaatttgataaactgacaaaagaagttgagcgtgtccaaaagaaacttggaaatgaaggatttatgaaaaaagcgcctgcacacgtaattgatgaagaacgtgaaaaagaaaaagattatgtggcgaagcgtgacgctgttcaaaaacgaatggctgagctgaagggt

SEQ ID NO: 51 - DNA, 1431 bp, B. subtilis
  glutamyl-tRNA amidotransferase, subunit B
ttgaactttgaaacggtaatcggacttgaagtccacgttgagttaaaaacaaaatcaaaaattttctcaagctctccaacgccattcggcgcggaggcgaatacgcagacaagcgttattgacctcggatatccgggcgtcctgcctgttctgaacaaagaagccgttgaattcgcaatgaaagccgctatggcgctcaactgtgagatcgcaacggatacgaagtttgaccgcaaaaactatttctatcctgacaacccgaaagcgtatcagatttctcaatttgataagccaatcggcgaaaacggctggatcgaaattgaagtcggcggcaaaacaaaacgcatcggcatcacgcgccttcatcttgaagaggatgccggaaaactgacgcatacgggcgacggctattctcttgttgacttcaaccgtcaaggaacgccgcttgttgagatcgtatcagagccggacatccgcacgccggaagaagcgtacgcatatcttgaaaagctgaaatccatcatccaatatacaggcgtttctgactgtaaaatggaagaaggctcacttcgctgtgacgccaatatctctcttcgtccgatcggccaagaggaattcggcacaaaaacagaattgaaaaacttgaactcctttgcgtttgttcaaaaaggccttgagcatgaagaaaaacgccaggagcaggttcttctttccggcggcgtcatccagcaagaaactcgccgttatgatgaagcaacgaagaaaaccattcttatgcgtgtcaaagagggatctgacgactaccgttacttcccagagccagatctagtcgagctctacattgatgatgaatggaaggaacgcgtaaaagcaagcattcctgagcttccggatgagcgccgcaagcgttatatcgaagagcttggcttgcctgcatatgacgcaatggttctgacgctgacaaaagaaatggctgatttcttcgaagaaaccgttcaaaaaggcgctgaagctaaacaagcgtctaactggctgatgggtgaagtgtcagcttacctaaacgcagaacaaaaagagcttgccgatgttgccctgacacctgaaggccttgccggcatgatcaaattgattgaaaaaggaaccatttcttctaagatcgcgaagaaagtgtttaaagaattgattgaaaaaggcggcgacgctgagaagattgtgaaagagaaaggccttgttcagatttctgacgaaggcgtgcttctgaagcttgtcactgaggcgcttgacaacaatcctcaatcaatcgaagactttaaaaacggaaaagaccgcgcgatcggcttcctagtcggacagattatgaaagcgtccaaaggacaagccaacccgccgatggtcaacaaaattctgcttgaagaaattaaaaaacgctaa

SEQ ID NO: 52 - DNA, 291 bp, B. subtilis
  glutamyl-tRNA amidotransferase, subunit C
atgtcacgaatttcaatagaagaagtaaagcacgttgcgcaccttgcaagacttgcgattactgaagaagaagcaaaaatgttcactgaacagctcgacagtatcatttcatttgccgaggagcttaatgaggttaacacagacaatgtggagcctacaactcacgtgctgaaaatgaaaaatgtcatgagagaagatgaagcgggtaaaggtcttccggttgaggatgtcatgaaaaatgcgcctgaccataaagacggctatattcgtgtgccatcaattctggactaa

SEQ ID NO: 53 - DNA, 31 bp, artificial
  primer
gtgcctcgaggattaagcattggtaactgtc

SEQ ID NO: 54 - DNA, 34 bp, artificial
  primer
tattctcgagacattaacctagaaagcactaagg

SEQ ID NO: 55 - DNA, 30 bp, artificial
  primer
ctgcctgtctcgaggctgatagcagttatc

SEQ ID NO: 56 - DNA, 30 bp, artificial
  primer
caactcgaggctggtcggacaaacacctag

SEQ ID NO: 57 - DNA, 30 bp, artificial
  primer
tgcgaattccggatgtaaggagaacggctc

SEQ ID NO: 58 - DNA, 32 bp, artificial
  primer
cttgtcgacgtacatcagaaatcgtacattcg

SEQ ID NO: 59 - DNA, 30 bp, artificial
  primer
ggtggatcccctcgccggcaatagttaccc

SEQ ID NO: 60 - DNA, 31 bp, artificial
  primer
aatctagactttcgttatacaaattttaacc

SEQ ID NO: 61 - DNA, 31 bp, artificial
  primer
tgtaggatccaatatgggcagaaaacacatg

SEQ ID NO: 62 - DNA, 31 bp, artificial
  primer
atatctagattccactctgtgtaccagtagc

SEQ ID NO: 63 - DNA, 20 bp, artificial
  primer
gttagctccttcggtcctcc

SEQ ID NO: 64 - DNA, 20 bp, artificial
  primer
gtaactggcttcagcagagc

SEQ ID NO: 65 - DNA, 21 bp, artificial
  primer
ctgccacatcgtctaggctgc

SEQ ID NO: 66 - DNA, 20 bp, artificial
  primer
caggagtccaaataccagag

SEQ ID NO: 67 - DNA, 20 bp, artificial
  primer
cccttgcctatactgtggac

SEQ ID NO: 68 - DNA, 20 bp, artificial
  primer
gttacaatagcgacggagag

SEQ ID NO: 69 - DNA, 27 bp, artificial
  primer
gtctgaattcgaggaaggtttacaccg

SEQ ID NO: 70 - DNA, 26 bp, artificial
  primer
ctgcgaattcatcttcatggtgaacc

SEQ ID NO: 71 - DNA, 20 bp, artificial
  primer
cattgtcattagttggctgg

SEQ ID NO: 72 - DNA, 28 bp, artificial
  primer
gcaagaattcaaaatccatcttcatcgg

SEQ ID NO: 73 - DNA, 29 bp, artificial
  primer
gtgactgaggatccgctagttccagatcg

SEQ ID NO: 74 - DNA, 30 bp, artificial
  primer
ctcttggatcccttcatcattctatcacac

SEQ ID NO: 75 - DNA, 25 bp, artificial
  primer
gcagattgggatggagttcccgtac

SEQ ID NO: 76 - DNA, 24 bp, artificial
  primer
ttacagcttcacaaagcggatttc

SEQ ID NO: 77 - DNA, 26 bp, artificial
  primer
tgctgcaagagctgtcggaaataaag

SEQ ID NO: 78 - DNA, 24 bp, artificial
  primer
aagaagaaaagaaagaagctaaag

SEQ ID NO: 79 - DNA, 31 bp, artificial; identical to SEQ ID NO: 53
  primer

SEQ ID NO: 80 - DNA, 34 bp, artificial; identical to SEQ ID NO: 54
  primer

SEQ ID NO: 81 - DNA, 32 bp, artificial
  primer
ctgctatgaattcgagtagtacacgtttcacc

SEQ ID NO: 82 - DNA, 28 bp, artificial
  primer
ctgaattctggtcggacaaacacctagc

SEQ ID NO: 83 - DNA, 29 bp, artificial
  primer
cggagaattctccgttacaaacgtcagag

SEQ ID NO: 84 - DNA, 29 bp, artificial
  primer
actgaattctggtcggacaaacacctagc

SEQ ID NO: 85 - DNA, 30 bp, artificial
  primer
ctcgaggctgaattcagttatcatttaagg

SEQ ID NO: 86 - DNA, 31 bp, artificial
  primer
gggaattcggacaaacacctagctttttgcg

SEQ ID NO: 87 - DNA, 1394 bp, artificial
  DNA sequence
gaattcgagtagtacacgtttcacctgtttgaagagagtctgcggtgctgggagcagataacgggcaatgtggaatggacttcggagcagctgaccgaactggaaaaaagtaggctcagccggagcagtctccgttacaaacgtcagagtgattccattttaatggaataatcagggtggtaccacggttcattcgtcccttttttacaggggaagaatgagccttttttattatgttttaagaaatggggttgatgttttcttgaaacaaacgattttttcaggcattcagccaagcggctcagtgacgctcggcaactatatcggtgcaatgaagcagtttgtcgaactgcagcatgattataacagctatttttgcatcgtcgatcagcatgcgataactgttcctcaagaccggcttgagcttagaaagaatatccgcaatctcgcggcgctttacttagctgtcggacttgatccagaaaaagcaacattgtttattcagtcagaggtccccgcacatgcgcaggccggatggatgatgcagtgtgtcgcctatatcggcgagcttgagcggatgactcaatttaaggacaaatccaaaggcaatgaagctgtcgtctccggcctgttaacatatccgccgctgatggccgctgatattctgctgtacggaacggatcttgtacctgtcggcgaggatcaaaagcagcaccttgagctgacgcggaatcttgcagaacgcttcaacaaaaaatacaacgacatctttacgattccggaagtgaaaattccaaaagtcggtgcacgtatcatgtctctgaatgatccgctgaagaaaatgagcaaatctgatccgaatcagaaagcttatattacattgctggatgagccgaagcagcttgaaaagaaaatcaaaagcgcagtaacggattctgaaggcattgtcaaatttgataaggaaaacaaaccgggcgtttccaaccttcttacaatttattcaatcctcggcaatacgacaattgaagagcttgaagcaaagtacgaaggaaaaggctacggcgagtttaaaggtgatttggcagaagtcgtagtgaacgcattaaaaccgatccaggaccgctattacgagctgatagaatctgaagaattagaccggattcttgatgaaggcgcggaacgagcgaatcggacagcaaacaaaatgctgaaaaaaatggagaatgccatgggtcttggaagaaaaagacgctaatcaaaaaaccgctctttgcaaagagcggtttttttcagttgacctttgattcgttttccatttcccaaagcttttcgaaaaaaggctgtccttttattaggttttcgcaaaaagctaggtgtttgtccgaccagaattc

SEQ ID NO: 88 - DNA, 1270 bp, artificial
  DNA sequence
gaattctccgttacaaacgtcagagtgattccattttaatggaataatcagggtggtaccacggttcattcgtcccttttttacaggggaagaatgagccttttttattatgttttaagaaatggggttgatgttttcgtgaaacaaacgattttttcaggcattcagccaagcggctcagtgacgctcggcaactatatcggtgcaatgaagcagtttgtcgaactgcagcatgattataacagctatttttgcatcgtcgatcagcatgcgataactgttcctcaagaccggcttgagcttagaaagaatatccgcaatctcgcggcgctttacttagctgtcggacttgatccagaaaaagcaacattgtttattcagtcagaggtccccgcacatgcgcaggccggatggatgatgcagtgtgtcgcctatatcggcgagcttgagcggatgactcaatttaaggacaaatccaaaggcaatgaagctgtcgtctccggcctgttaacatatccgccgctgatggccgctgatattctgctgtacggaacggatcttgtacctgtcggcgaggatcaaaagcagcaccttgagctgacgcggaatcttgcagaacgcttcaacaaaaaatacaacgacatctttacgattccggaagtgaaaattccaaaagtcggtgcacgtatcatgtctctgaatgatccgctgaagaaaatgagcaaatctgatccgaatcagaaagcttatattacattgctggatgagccgaagcagcttgaaaagaaaatcaaaagcgcagtaacggattctgaaggcattgtcaaatttgataaggaaaacaaaccgggcgtttccaaccttcttacaatttattcaatcctcggcaatacgacaattgaagagcttgaagcaaagtacgaaggaaaaggctacggcgagtttaaaggtgatttggcagaagtcgtagtgaacgcattaaaaccgatccaggaccgctattacgagctgatagaatctgaagaattagaccggattcttgatgaaggcgcggaacgagcgaatcggacagcaaacaaaatgctgaaaaaaatggagaatgccatgggtcttggaagaaaaagacgctaatcaaaaaaccgctctttgcaaagagcggtttttttcagttgacctttgattcgttttccatttcccaaagcttttcgaaaaaaggctgtccttttattaggttttcgcaaaaagctaggtgtttgtccgaccagaattc

SEQ ID NO: 89 - DNA, 1531 bp, artificial
  DNA sequence
gaattcagttatcatttaaggattttacagcactcgtgttatttatattattacaattttactggaaaaaagaatgttttttcatgatgacaattggttataattgatttataatggagcaatcaaaagaaaagcttgctatgaaaaagagtagtacacgtttcacctgtttgaagagagtctgcggtgctgggagcagataacgggcaatgtggaatggacttcggagcagccgaccgaactggaaaaaagtaggctcagccggagcggtctccgttacaaacgtcagagtgattccattttaatggaataatcagggtggtaccacggttcattcgtcccttttttacaggggaagaatgagccttttttattatgttttaagaaatggggttgatgttttcatgaaacaaacgattttttcaggcattcagccaagcggctcagtgacgctcggcaactatatcggtgcaatgaagcagtttgtcgaactgcagcatgattataacagctatttttgcatcgtcgatcagcatgcgacaaccgttcctcaagaccggcttgagcttagaaagaatatccgcaatctcgcggcgctttacttagctgtcggacttgatccagaaaaagcaacattgtttattcagtcagaggtccccgcacatgcgcaggccggatggatgatgcagtgtgtcgcctatatcggcgagcttgagcggatgactcaatttaaggacaaatccaaaggcaatgaagctgtcgtctccggcctgttaacatatccgccgctgatggccgctgatattctgctgtacggaacggatcttgtacctgtcggcgaggatcaaaagcagcaccttgagctgacgcggaatcttgcagaacgcttcaacaaaaaatacaacgacatctttacgattccggaagtgaaaattccaaaagtcggtgcacgtatcatgtctctgaatgatccgctgaagaaaatgagcaaatctgatccgaatcagaaagcttatattacattgctggatgagccgaagcagcttgaagagaaaatcaagagcgcagtaacggattctgaaggcattgtcaaatttgataaggaaaacaaaccgggcgtttccaaccttcttacaatttattcaatcctcggcaatacgacaattgaagagcttgaagcaaagtacgaaggaaaaggctacggcgagtttaaaggtgatttggcagaagtcgtagtgaacgcattagaaccgatccaggaccgctattacgagctgatagaatctgaagaattagaccggattcttgatgaaggcgcggaacgagcgaatcggacagcaaacaaaatgctgaaaaaaatggagaacgccatgggtcttggaagaaaaagacgctaatcgaaaaaccgctctttgcaaagggcggtttttttcagttgacctttgattcgttttccatctcccaaagcttttcgaaaaaaggctgtccttttattaggttctcgcaaaaagctaggtgtttgtccgaattc